(19) United States
(12) Patent Application Publication
(10) Pub. No.: US 2024/0215914 A1
(43) Pub. Date: Jul. 4, 2024

(54) APPARATUSES, SYSTEMS, AND METHODS OF ADAPTORS FOR MEDICAL DEVICES

(71) Applicant: DexCom, Inc., San Diego, CA (US)

(72) Inventors: Subhan Khan, Dallas, TX (US); Patrick J. Castagna, Escondido, CA (US); Scott Fall, San Diego, CA (US); Neal D. Johnston, San Diego, CA (US); Oleg Vladimirovich Los, San Diego, CA (US); Warren M. Terry, Poway, CA (US)

(21) Appl. No.: 18/400,603

(22) Filed: Dec. 29, 2023

Related U.S. Application Data
(60) Provisional application No. 63/436,527, filed on Dec. 31, 2022.

(51) Int. Cl. A61B 5/00; A61B 5/145

(52) U.S. Cl. CPC A61B 5/6849; A61B 5/14503; A61B 5/14532; A61B 2560/0443; A61B 2560/063

(57) ABSTRACT

The present examples relate generally to apparatuses, systems, and methods for deploying a medical device to skin of a host. The medical device may comprise a transcutaneous analyte sensor applied to the skin of a host. The apparatuses, systems, and methods may include adaptor bodies for interfacing with at least a portion of an on-skin wearable medical device and an applicator of the on-skin wearable medical device.

### CROSS REFERENCE TO RELATED APPLICATIONS

The present application claims the benefit of U.S. Provisional Patent Application No. 63/436,527, filed Dec. 31, 2022, the entire contents of which are incorporated herein by reference.

### BACKGROUND OF THE INVENTION

### Field of the Invention

Medical device apparatuses, systems, and methods. More particularly, apparatuses, systems, and methods are provided for adaptors for medical devices, which may include a transcutaneous analyte sensor for deployment to the skin of a host.

### Description of the Related Technology

Diabetes mellitus is a disorder in which the pancreas cannot create sufficient insulin (Type 1 or insulin dependent) and/or in which insulin is not effective (Type 2 or non-insulin dependent). In the diabetic state, the victim suffers from high blood sugar, which can cause an array of physiological derangements associated with the deterioration of small blood vessels, for example, kidney failure, skin ulcers, or bleeding into the vitreous of the eye. A hypoglycemic reaction (low blood sugar) can be induced by an inadvertent overdose of insulin, or after a normal dose of insulin or glucose-lowering agent accompanied by extraordinary exercise or insufficient food intake.

Conventionally, a person with diabetes carries a self-monitoring blood glucose (SMBG) monitor, which typically requires uncomfortable finger pricking methods. Due to the lack of comfort and convenience, a person with diabetes normally only measures his or her glucose levels two to four times per day. Unfortunately, such time intervals are spread so far apart that the person with diabetes likely finds out too late of a hyperglycemic or hypoglycemic condition, sometimes incurring dangerous side effects. Glucose levels may be alternatively monitored continuously by a measurement system including an on-skin sensor assembly. The sensor assembly may have a wireless transmitter which transmits measurement data to a receiver which can process and display information based on the measurements.

An applicator may be utilized to deploy an on-skin sensor assembly to a person. The application process should result in the on-skin sensor assembly being attached to the person in a state where it is capable of sensing the analyte (e.g., glucose) level information, communicating the sensed data to the transmitter, and transmitting the analyte level information to the receiver.

Exemplary systems are disclosed in, e.g., U.S. Patent Publication No. 2014/0088389, U.S. Patent Publication No. 2013/0267813, and U.S. Patent Publication No. 2018/0368771, owned by the assignee of the present application and herein incorporated by reference in their entireties.

This Background is provided to introduce a brief context for the Summary and Detailed Description that follow. This Background is not intended to be an aid in determining the scope of the claimed subject matter nor be viewed as limiting the claimed subject matter to implementations that solve any or all of the disadvantages or problems presented above.

### SUMMARY

The present systems and methods relate to apparatuses, systems, and methods for medical devices. More particularly, apparatuses, systems, and methods are provided for adaptors for medical devices, which may include a transcutaneous analyte sensor for deployment to the skin of a host. The various examples of the present apparatuses, systems, and methods may have several features, no single one of which is solely responsible for their desirable attributes. Without limiting the scope of the present examples as expressed by the claims that follow, their more prominent features now will be discussed briefly. After considering this discussion, and particularly after reading the section entitled “Detailed Description,” one will understand how the features of the present examples provide the advantages described herein.

In a first aspect, an apparatus comprising: an adaptor body configured to interface between at least a portion of an on-skin wearable medical device and an applicator of the on-skin wearable medical device.

Implementations of the embodiments may include one or more of the following. The adaptor body may be configured to interface between at least the portion of the on-skin wearable medical device having a first configuration and a portion of the applicator configured to engage a second configuration of at least a portion of an on-skin wearable medical device that is different than the first configuration. The second configuration may differ from the first configuration in one or more of a shape or a size. At least the portion with the first configuration may be a first on-skin wearable medical device, and the on-skin wearable medical device having at least the portion with the second configuration is a second on-skin wearable medical device, the first configuration comprises a configuration of a first wearable housing of the first on-skin wearable medical device, and the second configuration comprises a configuration of a second wearable housing of the second on-skin wearable medical device. The on-skin wearable medical device having at least the portion with the first configuration may be the same on-skin wearable medical device that has at least the portion with the second configuration. The on-skin wearable medical device may be a first on-skin wearable medical device, and the adaptor body may be configured to interface between a wearable housing of the first on-skin wearable medical device that is smaller than a wearable housing of a second on-skin wearable medical device that the applicator is configured to engage. The wearable housing of the first on-skin wearable medical device may have a smaller diameter or a smaller height than the wearable housing of the second on-skin wearable medical device. The adaptor body may be configured to adapt at least the portion of the on-skin wearable medical device to fit an engagement portion of the applicator. The adaptor body may include a retention area for receiving at least the portion of the on-skin wearable medical device. The adaptor body may include a cavity for receiving at least the portion of the on-skin wearable medical device. The adaptor body may include one or more walls bounding a retention area for receiving at least the portion of the on-skin wearable medical device. The one or more walls may include one or more side walls bounding the retention area. The one or more side walls may include an inner surface and an outer surface, the inner surface configured to face towards the portion of the on-skin wearable medical device and the outer surface configured to face opposite the inner surface. The one or more side walls may have a spacing between the inner surface and the outer surface that is configured to space an outer surface of the on-skin wearable medical device from the outer surface of the one or more side walls. The spacing may be configured to adapt at least the portion of the on-skin wearable medical device to fit an engagement portion of the applicator. The inner surface of the one or more side walls may form an inner perimeter having a different contour than an outer perimeter formed by the outer surface of the one or more side walls. The inner surface of the one or more side walls may form an inner perimeter having a smaller diameter than an outer perimeter formed by the outer surface of the one or more side walls. The one or more walls may include an upper wall bounding the retention area. The upper wall may include an inner surface and an outer surface, the inner surface configured to face towards the portion of the on-skin wearable medical device and the outer surface configured to face opposite the inner surface. The upper wall may have a spacing between the inner surface and the outer surface that is configured to space an outer surface of the on-skin wearable medical device from the outer surface of the upper wall. The spacing may be configured to adapt at least the portion of the on-skin wearable medical device to fit an engagement portion of the applicator. One or more stabilizers may be for stabilizing at least the portion of the on-skin wearable medical device within the retention area. The one or more stabilizers may comprise one or more protrusions for contacting at least the portion of the on-skin wearable medical device. The adaptor body may comprise a ring extending about a retention area for receiving at least the portion of the on-skin wearable medical device. The adaptor body may include a retainer portion for retaining at least the portion of the on-skin wearable medical device to the adaptor body. One or more device couplers may be for coupling the on-skin wearable medical device to the adaptor body. The one or more device couplers may comprise an adhesive. The one or more device couplers may comprise one or more of a protrusion or a recess configured to engage at least the portion of the on-skin wearable medical device. The one or more device couplers may be configured to deflect. The one or more device couplers may comprise releasable couplers configured to release the on-skin wearable medical device from the adaptor body. The one or more device couplers may comprise one or more arms. The one or more arms may bound a retention area for receiving at least the portion of the on-skin wearable medical device. The one or more arms may be configured to deflect radially outward from a retention area for receiving at least the portion of the on-skin wearable medical device. The adaptor body may include one or more support portions configured to support the one or more device couplers in a coupled configuration with at least the portion of the on-skin wearable medical device. The one or more support portions may comprise one or more contact surfaces of the adaptor body. The one or more support portions may comprise one or more hooks. One or more applicator couplers may be for coupling the adaptor body to at least a portion of the applicator. The one or more applicator couplers may comprise one or more of a protrusion or a recess configured to engage at least the portion of the applicator. The adaptor body may include an ejection portion configured to allow at least the portion of the on-skin wearable medical device to eject from the adaptor body. The ejection portion may comprise one or more openings in a surface of the adaptor body. The on-skin wearable medical device may include a transcutaneous analyte sensor, and the adaptor body is configured to be positioned within a housing of the applicator. The adaptor body may include an activation body configured to electrically activate the on-skin wearable medical device. The activation body may comprise a magnet. The adaptor body may include one or more walls bounding a retention area for receiving at least the portion of the on-skin wearable medical device, and the activation body is disposed on at least one of the one or more walls. The one or more walls may include an upper wall bounding the retention area, and the activation body is disposed on the upper wall.

In a second aspect, a system comprising: an applicator of an on-skin wearable medical device; and an adaptor body configured to interface between at least a portion of the on-skin wearable medical device and the applicator of the on-skin wearable medical device.

Implementations of the embodiments may include one or more of the following. The applicator may include an engagement portion configured to engage the adaptor body. The engagement portion may be configured to engage an outer surface of the adaptor body. The on-skin wearable medical device may be a first on-skin wearable medical device, and the engagement portion is configured to engage a second on-skin wearable medical device having a different configuration than the first on-skin wearable medical device. The first on-skin wearable medical device may have a wearable housing with a smaller diameter or a smaller height than a wearable housing of the second on-skin wearable medical device. The on-skin wearable medical device may be a first on-skin wearable medical device having a first wearable housing, and the engagement portion is sized to fit a second wearable housing of a second on-skin wearable medical device, the second wearable housing having a greater diameter or a greater height than the first wearable housing. The adaptor body may be configured to adapt at least the portion of the on-skin wearable medical device to fit the engagement portion of the applicator. The adaptor body may include a retention area for receiving at least the portion of the on-skin wearable medical device. The adaptor body may include a cavity for receiving at least the portion of the on-skin wearable medical device. The adaptor body may include one or more walls bounding a retention area for receiving at least the portion of the on-skin wearable medical device. The applicator may include an engagement portion configured to engage at least a portion of the one or more walls. The adaptor body may include one or more device couplers for coupling the on-skin wearable medical device to the adaptor body. The one or more device couplers may comprise releasable couplers configured to release the on-skin wearable medical device from the adaptor body. The adaptor body may include one or more support portions configured to support the one or more device couplers in a coupled configuration with at least the portion of the on-skin wearable medical device; and the applicator may include one or more contact surfaces configured to abut the one or more support portions. The one or more support portions may comprise one or more hooks. The one or more contact surfaces may comprise at least one post configured to engage the one or more hooks, the at least one post configured to be withdrawn from the one or more hooks to allow the one or more device couplers to decouple from at least the portion of the on-skin wearable medical device. The applicator may be configured such that the at least one post is withdrawn during retraction of a needle for guiding a transcutaneous analyte sensor into a skin of a host. The adaptor body may include an ejection portion configured to allow at least the portion of the on-skin wearable medical device to eject from the adaptor body; and the applicator includes an ejection actuator configured to eject the on-skin wearable medical device from the adaptor body. The on-skin wearable medical device may comprise a coupling mismatch with the applicator. The on-skin wearable medical device may have a wearable housing having a different configuration than a configuration of a wearable housing that an engagement portion of the applicator is configured to fit. The wearable housing of the on-skin wearable medical device may have a smaller diameter or a smaller height than the configuration of the wearable housing that the engagement portion of the applicator is configured to fit. The on-skin wearable medical device may include a transcutaneous analyte sensor. The adaptor body may comprise an activation body for electrically activating the on-skin wearable medical device. The activation body may comprise a magnet.

In a third aspect, a method comprising: utilizing an applicator to apply an on-skin wearable medical device to a skin of a host, an adaptor body interfacing between at least a portion of the on-skin wearable medical device and the applicator.

Implementations of the embodiments may include one or more of the following. The on-skin wearable medical device may comprise a coupling mismatch with the applicator. The adaptor body may adapt at least the portion of the on-skin wearable medical device to fit an engagement portion of the applicator. The on-skin wearable medical device may be a first on-skin wearable medical device, and the applicator has an engagement portion configured to engage a second on-skin wearable medical device having a different configuration than the first on-skin wearable medical device. The engagement portion may be configured to engage a wearable housing of the second on-skin wearable medical device that has a greater diameter or a greater height than a wearable housing of the first on-skin wearable medical device. The method may include releasing the on-skin wearable medical device from the adaptor body. The method may include utilizing one or more releasable couplers to release the on-skin wearable medical device from the adaptor body. The method may include retaining the adaptor body to the applicator following release of the on-skin wearable medical device from the adaptor body. The on-skin wearable medical device may include a transcutaneous analyte sensor. The method may include guiding the transcutaneous analyte sensor into the skin of the host utilizing an insertion element of the applicator. The method may further comprise electrically activating the on-skin wearable medical device with an activation body disposed on the adaptor body. The activation body may comprise a magnet. Electrically activating the on-skin wearable medical device may comprise causing the on-skin wearable medical device to activate from a lower power state to a higher power state. Electrically activating the on-skin wearable medical device may include increasing a distance between the on-skin wearable medical device and the activation body. The adaptor body may include one or more walls bounding a retention area for receiving at least the portion of the on-skin wearable medical device, and the activation body is disposed on at least one of the one or more walls.

In further aspects and embodiments, the above methods and features of the various aspects are formulated in terms of a system as in various aspects, having an applicator configured to carry out the method features. Any of the features of an embodiment of any of the aspects, including but not limited to any embodiments of any of the first through third aspects referred to above, is applicable to all other aspects and embodiments identified herein, including but not limited to any embodiments of any of the first through third aspects referred to above. Moreover, any of the features of an embodiment of the various aspects, including but not limited to any embodiments of any of the first through third aspects referred to above, is independently combinable, partly or wholly with other embodiments described herein in any way, e.g., one, two, or three or more embodiments may be combinable in whole or in part. Further, any of the features of an embodiment of the various aspects, including but not limited to any embodiments of any of the first through third aspects referred to above, may be made optional to other aspects or embodiments. Any aspect or embodiment of a method can be performed by a system or apparatus of another aspect or embodiment, and any aspect or embodiment of a system or apparatus can be configured to perform a method of another aspect or embodiment, including but not limited to any embodiments of any of the first through third aspects referred to above.

This Summary is provided to introduce a selection of concepts in a simplified form. The concepts are further described in the Detailed Description section. Elements or steps other than those described in this Summary are possible, and no element or step is necessarily required. This Summary is not intended to identify key features or essential features of the claimed subject matter, nor is it intended for use as an aid in determining the scope of the claimed subject matter. The claimed subject matter is not limited to implementations that solve any or all disadvantages noted in any part of this disclosure.

### BRIEF DESCRIPTION OF THE DRAWINGS

These and other features, aspects, and advantages are described below with reference to the drawings, which are intended to illustrate, but not to limit, the disclosure. In the drawings, like reference characters denote corresponding features consistently throughout similar examples.

FIG. 1 illustrates a schematic view of a continuous analyte sensor system.

FIG. 2A illustrates a top perspective assembly view of an on-skin sensor assembly.

FIG. 2B illustrates a bottom perspective view of the on-skin sensor assembly of FIG. 2A in an assembled state.

FIG. 2C illustrates a top perspective view of the on-skin sensor assembly of FIG. 2A in an assembled state.

FIG. 3 illustrates a perspective assembly view of an on-skin sensor assembly.

FIG. 4 illustrates a perspective view of an on-skin sensor assembly.

FIG. 5 illustrates a perspective view of an applicator system for an on-skin sensor assembly of an analyte sensor system.

FIG. 6 illustrates an exploded perspective view of the applicator system of FIG. 5.

FIGS. 7-9 illustrate several cross-sectional views of the applicator system of FIGS. 5 and 6, taken along the section line A-A′ of FIG. 5, during operation.

FIGS. 10-12 illustrate several cross-sectional views of the applicator system of FIGS. 5 and 6, taken along the section line B-B′ of FIG. 5, during operation.

FIGS. 13 and 14 illustrate magnified views of some features of the applicator system of FIGS. 5 and 6.

FIGS. 15 and 16 illustrate magnified views of some features of the applicator system of FIGS. 5 and 6.

FIG. 17 illustrates a perspective partial cutaway view of the needle carrier assembly, hub, and on-skin sensor assembly of the applicator system of FIGS. 5 and 6.

FIG. 18 illustrates a cross-sectional view of the hub and on-skin sensor assembly of the applicator system of FIGS. 5 and 6.

FIG. 19 illustrates a top view of a portion of the needle carrier assembly and hub of FIGS. 5 and 6.

FIGS. 20A and 20B illustrate perspective views of locking features for needles for use in an applicator for an analyte sensor system.

FIGS. 21-23 illustrate several cross-sectional views, and various features and operating positions, of yet another applicator for an on-skin sensor assembly of an analyte sensor system.

FIG. 24 illustrates a perspective view of various features of the applicator system of FIGS. 21-23.

FIG. 25 illustrates a cross-sectional view of a system, according to some examples.

FIGS. 26A-26B illustrate cross-sectional schematic views of an analyte sensor with and without an insertion element.

FIG. 27 illustrates a perspective view of a housing of an on-skin sensor assembly.

FIG. 28 illustrates a perspective view of a housing of an on-skin sensor assembly.

FIG. 29A illustrates an upper perspective view of an adaptor.

FIG. 29B illustrates a lower perspective view of the adaptor shown in FIG. 29A.

FIG. 29C illustrates a top view of the adaptor shown in FIG. 29A, positioned upon an on-skin sensor assembly.

FIG. 30A illustrates a perspective view of the adaptor shown in FIG. 29A coupled to an applicator.

FIG. 30B illustrates a perspective view of the adaptor shown in FIG. 29A coupled to an applicator and coupled to an on-skin sensor assembly.

FIG. 30C illustrates a perspective detail view of a device coupler of an adaptor.

FIG. 30D illustrates a perspective view of a proximal movement of the adaptor and on-skin sensor assembly shown in FIG. 30B.

FIG. 30E illustrates a perspective view of retraction of an engagement portion of an applicator.

FIG. 30F illustrates a perspective view of the adaptor shown in FIG. 30A with an on-skin sensor assembly having been released from the adaptor.

FIG. 30G illustrates a perspective detail view of an applicator coupler of the adaptor shown in FIG. 29A.

FIG. 31A illustrates a top view of an adaptor.

FIG. 31B illustrates a top view of the adaptor shown in FIG. 31A, positioned upon an on-skin sensor assembly.

FIG. 31C illustrates a bottom view of the adaptor shown in FIG. 31A, positioned upon an on-skin sensor assembly.

FIG. 31D illustrates a top view of the adaptor shown in FIG. 31A, positioned upon an on-skin sensor assembly and with device couplers in a closed position.

FIG. 31E illustrates a bottom view of the adaptor shown in FIG. 31A, positioned upon an on-skin sensor assembly and with device couplers in a closed position.

FIG. 32A illustrates an end view of the adaptor shown in FIG. 31A, positioned upon an on-skin sensor assembly and coupled to an applicator.

FIG. 32B illustrates a perspective view of the adaptor shown in FIG. 31A, coupled to an applicator.

FIG. 32C illustrates an end view of retraction of an engagement portion of an applicator.

FIG. 32D illustrates a perspective detail view of release of a device coupler from an on-skin sensor assembly.

FIG. 32E illustrates an end view of an on-skin sensor assembly having been released from the adaptor shown in FIG. 31A.

FIG. 33A illustrates a top view of an adaptor positioned upon an on-skin sensor assembly.

FIG. 33B illustrates a bottom view of the adaptor shown in FIG. 33A positioned upon an on-skin sensor assembly.

FIG. 33C illustrates a bottom view of the adaptor shown in FIG. 33A separated from an on-skin sensor assembly.

FIG. 34A illustrates a perspective view of the adaptor shown in FIG. 33A, coupled to an applicator.

FIG. 34B illustrates a perspective view of a proximal movement of the adaptor and on-skin sensor assembly shown in FIG. 33B.

FIG. 34C illustrates a perspective view of the adaptor shown in FIG. 33A with an on-skin sensor assembly having been released from the adaptor.

FIG. 34D illustrates a side cross sectional view of the adaptor shown in FIG. 34A coupled to an on-skin sensor assembly and positioned within an applicator.

FIG. 34E illustrates a side cross sectional view of the adaptor shown in FIG. 34A positioned within an applicator.

FIG. 34F illustrates a perspective view of a portion of an applicator.

FIG. 35A illustrates a top view of an adaptor.

FIG. 35B illustrates a bottom perspective view of the adaptor shown in FIG. 35A.

FIG. 35C illustrates a top view of the adaptor shown in FIG. 35A, positioned upon an on-skin sensor assembly.

FIG. 35D illustrates a bottom view of the adaptor shown in FIG. 35A, positioned upon an on-skin sensor assembly.

FIG. 35E illustrates a bottom view of the adaptor shown in FIG. 35A, positioned upon an on-skin sensor assembly and coupled to an applicator.

FIG. 35F illustrates a perspective view of the adaptor shown in FIG. 35A, positioned upon an on-skin sensor assembly and coupled to an applicator.

FIG. 35G illustrates a detail perspective view of a contact surface of the applicator contacting support portions of the adaptor shown in FIG. 35A.

FIG. 36A illustrates a perspective view of an adaptor.

FIG. 36B illustrates a perspective view of an on-skin sensor assembly.

FIG. 36C illustrates a perspective view of the on-skin sensor assembly shown in FIG. 36B within a retention area of the adaptor shown in FIG. 36A.

FIG. 37 illustrates a cross sectional view along line C-C′ in FIG. 36C of the on-skin sensor assembly within the retention area of the adaptor.

FIG. 38 illustrates a perspective view of an engagement portion of an applicator.

FIG. 39A illustrates a cross sectional view of an engagement portion of an applicator coupled to the adaptor shown in FIG. 36A.

FIG. 39B illustrates a cross sectional view of an engagement portion of an applicator released from the adaptor shown in FIG. 36A.

FIG. 39C illustrates a cross sectional view of the adaptor shown in FIG. 36A released from the on-skin sensor assembly.

FIG. 40 illustrates a perspective view of an adaptor body shown with an on-skin sensor assembly.

FIG. 41 illustrates a side cross sectional view of the adaptor body and on-skin sensor assembly shown in FIG. 40 along line D-D′ of FIG. 40.

### DETAILED DESCRIPTION

The following description illustrates some examples of the disclosure in detail. Those of skill in the art will recognize that there are numerous variations and modifications of the disclosure that are encompassed by its scope. Accordingly, the description of a certain example should not be deemed to limit the scope of the present disclosure.

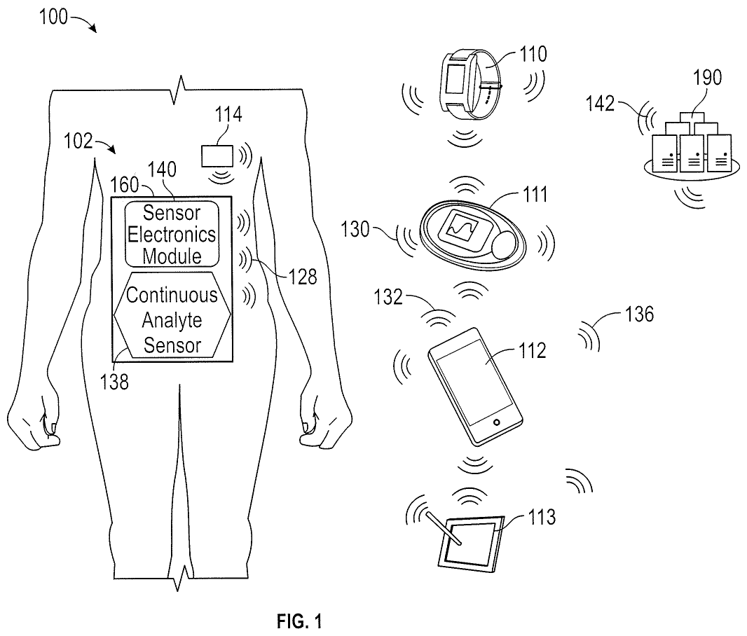

FIG. 1 is a diagram depicting an example medical device system according to examples herein. The medical device system in examples may comprise a continuous analyte monitoring system 100. The continuous analyte monitoring system 100 may include an analyte sensor system 102 comprising an on-skin sensor assembly 160 configured to be fastened to the skin of a host via a base (not shown).

In examples, other forms of medical device systems may be utilized, including other forms of monitoring systems, medicament delivery systems, or other therapeutic systems. In examples, an on-skin wearable medical device may be utilized that may comprise an on-skin sensor assembly, or a medicament delivery medical device, among other forms of on-skin wearable medical devices.

As shown in FIG. 1, the analyte sensor system 102 may be operatively connected to a host and a plurality of display devices 110-114 according to certain aspects of the present disclosure. Example display devices 110-114 may include computers such as smartphones, smartwatches, tablet computers, laptop computers, and desktop computers. In some examples, display devices 110-114 may be Apple Watches, iPhones, and iPads made by Apple Inc., or iOS, Windows, or Android operating system devices. It should be noted that display device 114 alternatively or in addition to being a display device, may be a medicament delivery device that can act cooperatively with analyte sensor system 102 to deliver medicaments to the host. Analyte sensor system 102 may include a sensor electronics module 140 and a continuous analyte sensor 138 associated with sensor electronics module 140. Sensor electronics module 140 may be in direct wireless communication with one or more of the plurality of display devices 110-114 via wireless communications signals. As will be discussed in greater detail below, display devices 110-114 may also communicate amongst each other and/or through each other to analyte sensor system 102. For ease of reference, wireless communications signals from analyte sensor system 102 to display devices 110-114 can be referred to as “uplink” signals 128. Wireless communications signals from, e.g., display devices 110-114 to analyte sensor system 102 can be referred to as “downlink” signals 130. Wireless communication signals between two or more of display devices 110-114 may be referred to as “crosslink” signals 132. Additionally, wireless communication signals can include data transmitted by one or more of display devices 110-113 via “long-range” uplink signals 136 (e.g., cellular signals) to one or more remote servers 190 or network entities, such as cloud-based servers or databases, and receive long-range downlink signals 142 transmitted by remote servers 190.

In examples shown by FIG. 1, one of the plurality of display devices may be a custom display device 111 specially designed for displaying certain types of displayable sensor information associated with analyte values received from the sensor electronics module 140 (e.g., a numerical value and an arrow, in some examples). In some examples, one of the plurality of display devices may be a handheld device 112, such as a mobile phone based on the Android, IOS operating systems or other operating system, a palm-top computer and the like, where handheld device 112 may have a relatively larger display and be configured to display a graphical representation of the continuous sensor data (e.g., including current and historic data). Other display devices can include other hand-held devices, such as a tablet 113, a smart watch 110, a medicament delivery device 114, a blood glucose meter, and/or a desktop or laptop computer.

It should be understood that in the case of display device 114, which may be a medicament delivery device in addition to or instead of a display device, the alerts and/or sensor information provided by continuous analyte sensor 138 vis-à-vis sensor electronics module 140, can be used to initiate and/or regulate the delivery of the medicament to host.

During use, a sensing portion of sensor 138 may be disposed under the host's skin and a contact portion of sensor 138 can be electrically connected to sensor electronics module 140. Electronics module 140 can be engaged with a housing (e.g., a base) which is attached to a patch that may engage the skin of the host. The housing may comprise a wearable housing. The patch may be an adhesive patch in examples. In some examples, electronics module 140 is integrally formed with the housing. Furthermore, electronics module 140 may be disposable and directly coupled to the patch.

Continuous analyte sensor system 100 can include a sensor configuration that provides an output signal indicative of a concentration of an analyte. The output signal including (e.g., sensor data, such as a raw data stream, filtered data, smoothed data, and/or otherwise transformed sensor data) is sent to the receiver.

In some examples, analyte sensor system 102 includes a transcutaneous glucose sensor, such as is described in U.S. Patent Publication No. 2011/0027127, the entire contents of which are hereby incorporated by reference. In some examples, sensor system 102 includes a continuous glucose sensor and comprises a transcutaneous sensor (e.g., as described in U.S. Pat. No. 6,565,509, as described in U.S. Pat. No. 6,579,690, and/or as described in U.S. Pat. No. 6,484,046). The contents of U.S. Pat. Nos. 6,565,509, 6,579,690, and 6,484,046 are hereby incorporated by reference in their entirety.

Various signal processing techniques and glucose monitoring system examples suitable for use with the examples described herein are described in U.S. Patent Publication No. 2005/0203360 and U.S. Patent Publication No. 2009/0192745, the contents of which are hereby incorporated by reference in their entirety. The sensor can extend through a housing, which can maintain sensor 138 on, in or under the skin and/or can provide for electrical connection of sensor 138 to sensor electronics in sensor electronics module 140.

In some examples, description of a base, a housing, a wearable, and/or a transmitter of on-skin sensor assembly 160 may be interchangeable. In other examples, a base and a housing of on-skin sensor assembly 160 may be different in the sense that they may be separate components from sensor electronics module 140, e.g., from a transmitter or receiver.

In several examples, sensor 138 is in a form of a wire. A distal end of the wire can be formed, e.g., having a conical shape (to facilitate inserting the wire into the tissue of the host). Sensor 138 may comprise an elongate analyte sensor, and may include an elongate conductive body, such as an elongate conductive core (e.g., a metal wire) or an elongate conductive core coated with one, two, three, four, five, or more layers of material, each of which may or may not be conductive. The elongate analyte sensor may be long and thin, yet flexible and strong. For example, in some examples, the smallest dimension of the elongate conductive body is less than 0.1 inches, less than 0.075 inches, less than 0.05 inches, less than 0.025 inches, less than 0.01 inches, less than 0.004 inches, less than 0.002 inches, less than 0.001 inches, and/or less than 0.0005 inches.

Sensor 138 may have a circular shaped cross section. In some examples, the cross section of the elongated conductive body can be ovoid, rectangular, triangular, polyhedral, star-shaped, C-shaped, T-shaped, X-shaped, Y-shaped, irregular, or the like. In some examples, a conductive wire electrode is employed as a core. In other examples, sensor 138 may be disposed on a substantially planar substrate. To such an electrode, one or two additional conducting layers may be added (e.g., with intervening insulating layers provided for electrical isolation). The conductive layers can be comprised of any suitable material. In certain examples, it may be desirable to employ a conductive layer comprising conductive particles (i.e., particles of a conductive material) in a polymer or other binder.

In some examples, the materials used to form the elongate conductive body (e.g., stainless steel, titanium, tantalum, platinum, platinum-iridium, iridium, certain polymers, and/or the like) can be strong and hard, and therefore can be resistant to breakage. For example, in several examples, the ultimate tensile strength of the elongated conductive body is greater than 80 kPsi and less than 140 kPsi, and/or the Young's modulus of the elongate conductive body is greater than 160 GPa and less than 220 GPa. The yield strength of the elongate conductive body can be greater than 58 kPsi and less than 2200 kPsi.

Electronics module 140 can be releasably or permanently coupled to sensor 138. Electronics module 140 can include electronic circuitry associated with measuring and processing the continuous analyte sensor data. Electronics module 140 can be configured to perform algorithms associated with processing and calibration of the sensor data. For example, electronics module 140 can provide various aspects of the functionality of a sensor electronics module as described in U.S. Patent Publication No. 2009/0240120 and U.S. Patent Publication No. 2012/0078071, the entire contents of which are incorporated by reference herein. Electronics module 140 may include hardware, firmware, and/or software that enable measurement of levels of the analyte via a glucose sensor, such as sensor 138.

For example, electronics module 140 can include a potentiostat, a power source for providing power to sensor 138, signal processing components, data storage components, and a communication module (e.g., a telemetry module) for one-way or two-way data communication between electronics module 140 and one or more receivers, repeaters, and/or display devices, such as devices 110-114. Electronic components can be affixed to a printed circuit board (PCB), or the like, and can take a variety of forms. The electronic components can take the form of an integrated circuit (IC), such as an Application-Specific Integrated Circuit (ASIC), a microcontroller, and/or a processor. The electronics module 140 may include sensor electronics that are configured to process sensor information, such as storing data, analyzing data streams, calibrating analyte sensor data, estimating analyte values, comparing estimated analyte values with time-corresponding measured analyte values, analyzing a variation of estimated analyte values, and the like. Examples of systems and methods for processing sensor analyte data are described in more detail in U.S. Pat. Nos. 7,310,544, 6,931,327, U.S. Patent Publication No. 2005/0043598, U.S. Patent Publication No. 2007/0032706, U.S. Patent Publication No. 2007/0016381, U.S. Patent Publication No. 2008/0033254, U.S. Patent Publication No. 2005/0203360, U.S. Patent Publication No. 2005/0154271, U.S. Patent Publication No. 2005/0192557, U.S. Patent Publication No. 2006/0222566, U.S. Patent Publication No. 2007/0203966 and U.S. Patent Publication No. 2007/0208245, the contents of which are hereby incorporated by reference in their entirety. Electronics module 140 may communicate with the devices 110-114, and/or any number of additional devices, via any suitable communication protocol. Example communication methods or protocols include radio frequency; Bluetooth; universal serial bus; any of the wireless local area network (WLAN) communication standards, including the IEEE 802.11, 802.15, 802.20, 802.22 and other 802 communication protocols; ZigBee; wireless (e.g., cellular) telecommunication; paging network communication; magnetic induction; satellite data communication; a proprietary communication protocol, open source communication protocol, and/or any suitable wireless communication method.

Additional sensor information is described in U.S. Pat. Nos. 7,497,827 and 8,828,201. The entire contents of U.S. Pat. Nos. 7,497,827 and 8,828,201 are incorporated by reference herein.

Any sensor shown or described herein can be an analyte sensor; a glucose sensor; and/or any other suitable sensor. A sensor described in the context of any example can be any sensor described herein or incorporated by reference. Sensors shown or described herein can be configured to sense, measure, detect, and/or interact with any analyte.

As used herein, the term “analyte” is a broad term, and is to be given its ordinary and customary meaning to a person of ordinary skill in the art (and is not to be limited to a special or customized meaning), and refers without limitation to a substance or chemical constituent in a biological fluid (for example, blood, interstitial fluid, cerebral spinal fluid, lymph fluid, urine, sweat, saliva, etc.) that can be analyzed. Analytes can include naturally occurring substances, artificial substances, metabolites, or reaction products.

In some examples, the analyte for measurement by the sensing regions, devices, systems, and methods is glucose. However, other analytes are contemplated as well, including, but not limited to ketone bodies; acetyl-CoA; acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; cortisol; testosterone; choline; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; triglycerides; glycerol; free ß-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); acetone (e.g., succinylacetone); acetoacetic acid; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain examples. The analyte can be naturally present in the biological fluid or endogenous, for example, a metabolic product, a hormone, an antigen, an antibody, and the like. Alternatively, the analyte can be introduced into the body or exogenous, for example, a contrast agent for imaging, a radioisotope, a chemical agent, a fluorocarbon-based synthetic blood, or a drug or pharmaceutical composition, including but not limited to insulin; glucagon; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), 5-hydroxyindoleacetic acid (FHIAA), and intermediaries in the Citric Acid Cycle.

Any of the features described in the context of at least FIG. 1 can be applicable to all aspects and examples identified herein. Moreover, any of the features of an example is independently combinable, partly or wholly with other examples described herein in any way, e.g., one, two, or three or more examples may be combinable in whole or in part. Further, any of the features of an example may be made optional to other aspects or examples. Any aspect or example of a method can be performed by a system or apparatus of another aspect or example, and any aspect or example of a system can be configured to perform a method of another aspect or example.

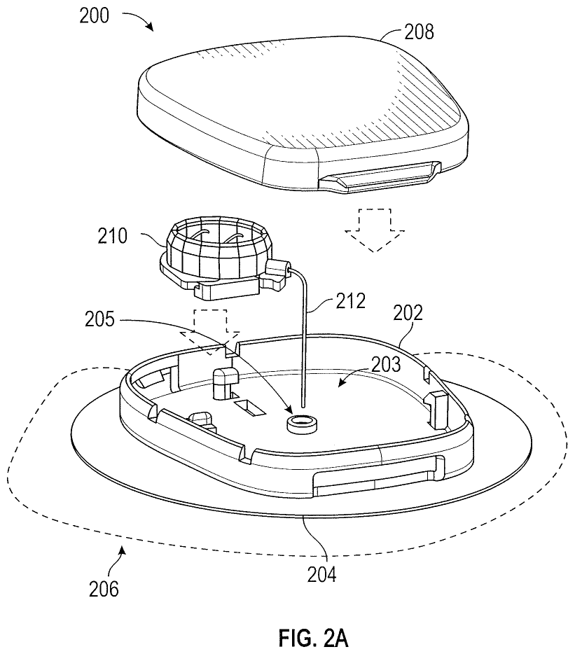

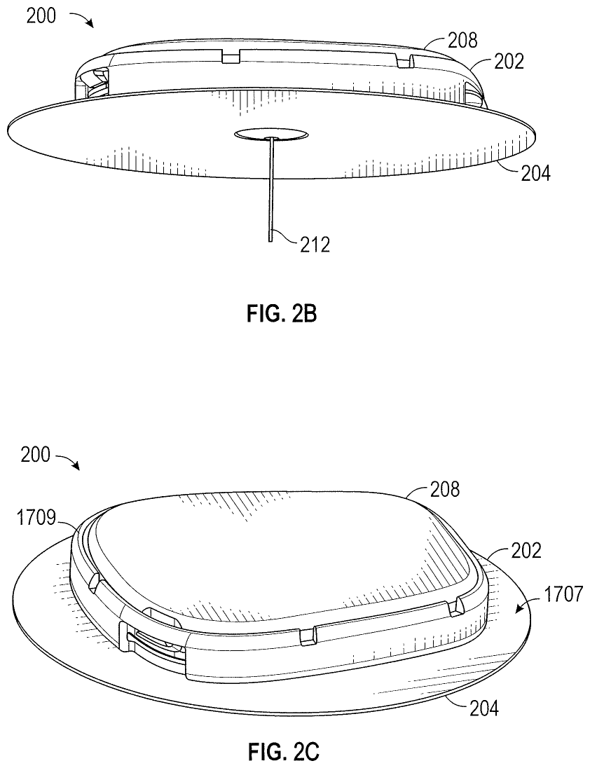

FIG. 2A illustrates a perspective view of an exemplary on-skin wearable medical device, in the form of an on-skin sensor assembly 200, which is configured to be deployed to skin. The on-skin sensor assembly 200 may include a housing or base 202. The housing or base 202 may be configured to be worn on skin of a host and may include a distal surface for facing towards the skin and a proximal surface 203 facing opposite the distal surface. The housing or base 202 may include an opening 205 for an insertion element to be retracted proximally through from the skin. A patch 204 such as an adhesive patch can couple the base 202 to the skin 206 of the host. The patch 204 may be positioned on the distal surface of the housing or base 202. In some examples, the adhesive patch 204 may include an engaging surface for engaging the skin and including an adhesive suitable for skin adhesion, for example a pressure sensitive adhesive (e.g., acrylic, rubber-based, or other suitable type) bonded to a carrier substrate (e.g., spun lace polyester, polyurethane film, or other suitable type) for skin attachment, though any suitable type of adhesive is also contemplated. An on-skin sensor assembly 200 may comprise an electronics unit 208 (e.g., a transmitter) which may further comprise a glucose sensor module 210 coupled to an analyte sensor such as a transcutaneous analyte sensor (e.g., a glucose sensor) 212 and to base 202.

The applicator system can engage the adhesive patch 204 to skin 206. The glucose sensor module 210 may be secured to base 202 (e.g., via retention elements such as snap fits and/or interference features, adhesive, welding, etc.) to ensure analyte sensor 212 (e.g., glucose sensor) is coupled to base 202. In alternative examples, the sensor module 210 and base 202 are preassembled or manufactured as a single component.

After on-skin sensor assembly 200 is deployed to a user's skin, a user (or an applicator) can couple electronics unit 208 (e.g., a transmitter) to on-skin sensor assembly 200 via retention elements such as snap fits and/or interference features. Electronics unit 208 can measure and/or analyze glucose indicators sensed by transcutaneous analyte sensor (e.g., a glucose sensor) 212. Electronics unit 208 can transmit information (e.g., measurements, analyte data, glucose data) to a remotely located device (e.g., 110-114 shown in FIG. 1).

On-skin sensor assembly 200 may be attached to the host with use of an applicator adapted to provide convenient and secure application. Such an applicator may also be used for attaching electronics unit 208 to base 202, inserting sensor 212 through the host's skin, and/or connecting sensor 212 to electronics unit 208. Once electronics unit 208 is engaged with the base and sensor 212 has been inserted into the skin (and is connected to the electronics unit 208), the sensor assembly can detach from the applicator.

FIG. 2B illustrates a perspective view of electronics unit 208 coupled to base 202 via retention elements such as snap fits and/or interference features. In some examples, electronics unit 208 and base 202 are coupled by adhesive, welding, or other bonding techniques. Patch 204, on a distal surface of base 202, is configured to couple sensor assembly 200 to the skin.

FIG. 2C illustrates a perspective view of on-skin sensor assembly 200. On-skin sensor assembly 200 may be disposable or reusable. FIG. 2C further illustrates electronics unit 208 coupled to a base 202, and adhesive patch 204 configured to be attached to on-skin sensor assembly 200, which, when combined, may be held within the applicator.

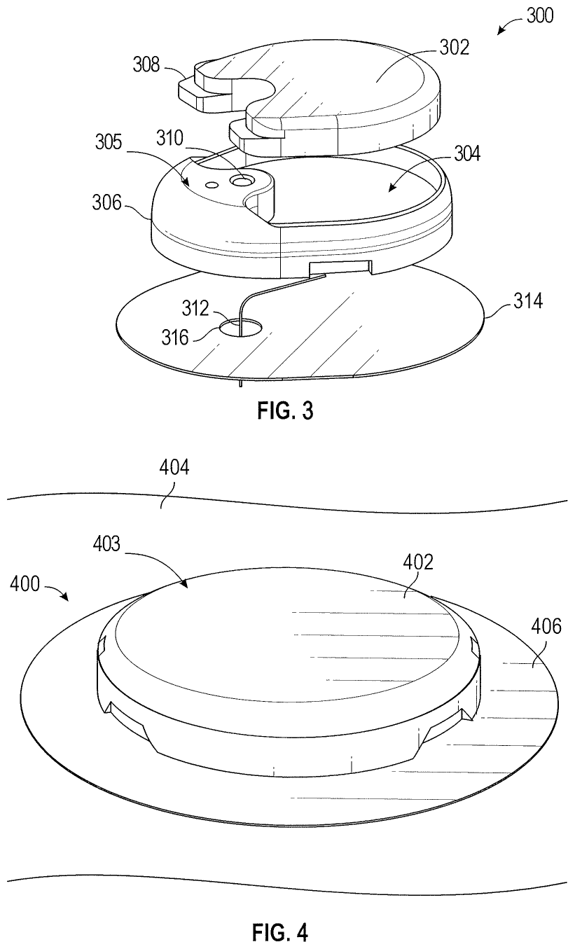

FIG. 3 illustrates an example of an on-skin wearable medical device in the form of an on-skin sensor assembly 300 with an electronics unit 302 configured to insert into a cavity 304 of the base or housing 306. The base or housing 306 may be configured to be worn on skin of a host and may include a distal surface for facing towards the skin and a proximal surface 305 facing opposite the distal surface. The electronics unit 302 may include one or more tabs 308 that couple to a portion of the housing 306 and allow the electronics unit 302 to be retained by the housing 306. The housing 306 may include an opening 310 for an insertion element to be retracted proximally through from the skin. The opening 310 may allow the insertion element (such as a needle) to pass through to deploy the transcutaneous analyte sensor 312 to the skin. The patch 314 may further include an aperture 316 that may allow the sensor 312 and the insertion element to pass through. The electronics unit 302 may couple to the housing 306 prior to or following deployment of the sensor 312 to the host's skin.

FIG. 4 illustrate an example of an on-skin wearable medical device in the form of an on-skin sensor assembly 400, in which the electronics unit is integral with the housing 402. The housing 402 may be configured to be worn on skin of a host and may include a distal surface for facing towards the skin and a proximal surface 403 facing opposite the distal surface. The on-skin sensor assembly 400 is shown on the skin 404, with the patch 406 engaging the skin 404.

The examples of FIGS. 2A-4 may each include an engaging surface for engaging the skin. The engaging surface may be positioned on the patch in examples, for example on a distal surface of the patch or may have another position in examples. The engaging surface may comprise an adhesive surface in examples configured to adhere to the skin. The adhesive can be configured for adhering to skin. Additional adhesive information is described in U.S. Pat. No. 11,219,413, which was filed on Aug. 25, 2015. The entire contents of U.S. Pat. No. 11,219,413 are incorporated by reference herein. The engaging surface in examples may be covered with a liner prior to deployment to the host's skin.

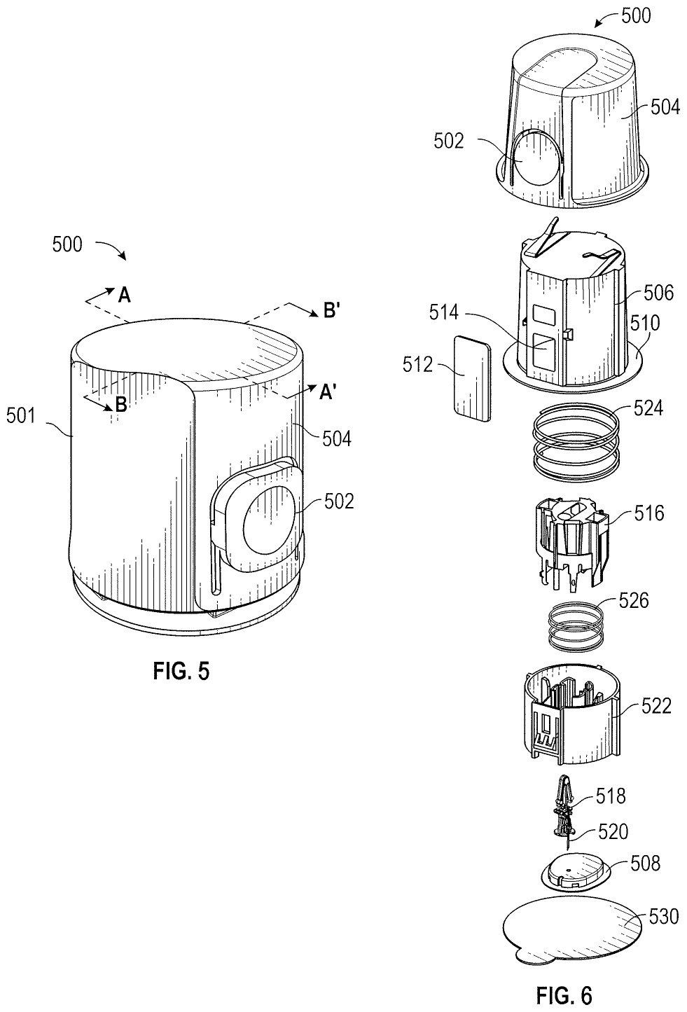

FIG. 5 illustrates a system for deploying an on-skin wearable medical device to skin. The system may comprise an applicator system in examples. The system may include an applicator for an on-skin sensor assembly of an analyte sensor system, according to some examples. In examples, other forms of systems may be utilized.

The applicator 500 may include an applicator housing 501, which may include an outer housing 504 and an inner housing 506, and other forms of housings in examples. The applicator housing 501 may be configured to retain the on-skin wearable medical device in examples. The applicator 500 may include a deployment mechanism that may be configured to deploy the on-skin wearable medical device to skin. The deployment mechanism, for example, may include an engagement portion for retaining the on-skin wearable medical device and releasing the on-skin wearable medical device from the applicator housing 501 to the skin in examples. The engagement portion may include one or more retention element(s). The deployment mechanism may include an insertion assembly for inserting at least a portion of the on-skin wearable medical device into the skin. The insertion assembly may drive a portion of the on-skin wearable medical device, such as the insertion element and the sensor, into the skin of the host. The deployment mechanism may include a retraction assembly for retracting the portion of the on-skin wearable medical device from the skin, such as an insertion element.

In examples, the applicator 500 may include an activation element 502 disposed on a side of applicator 500, for example, on a side of an outer housing 504 of applicator 500. In some examples, activation element 502 may be a button, a switch, a toggle, a slide, a trigger, a knob, a rotating member, a portion of applicator 500 that deforms and/or flexes or any other suitable mechanism for activating an insertion and/or retraction assembly of applicator 500. In some examples, activation element 502 may be disposed in any location, e.g., a top, upper side, lower side, or any other location of applicator 500. Applicator 500 may be large enough for a host to grasp with a hand and push, or otherwise activate, activation element 502 with, for example, a thumb, or with an index finger and/or a middle finger.

Applicator 500 may be configured with one or more safety features such that applicator 500 is prevented from activating until the safety feature is deactivated. In one example, the one or more safety features prevents applicator 500 from activating unless applicator 500 is pressed against the skin of a host with sufficient force. Moreover, as will be described in more detail in connection with one or more of FIGS. 6-20B below, applicator 500 may be further configured such that one or more components therein retract based at least in part on the one or more components pushing against the skin of the host with a force exceeding a predetermined threshold, rather than based on the one or more components translating beyond a predetermined and static distal position. In other words, applicator 500 may implement force-based retraction triggering rather than being limited to displacement-based retraction triggering.

FIG. 6 illustrates an exploded perspective view of applicator 500 of FIG. 5, according to some examples. Applicator 500 may include outer applicator housing 504 comprising activation element 502. The outer applicator housing 504 may be configured to be gripped by a user in examples. Outer applicator housing 504 may be configured to translate in a distal direction by a force applied by a host to applicator 500, specifically to inner housing 506, thereby aligning activation element 502 in a position that allows applicator 500 to fire. Further explanation of the alignment process will be explained below.

Applicator 500 further comprises inner housing 506, configured to house at least one or more mechanisms utilized to apply on-skin sensor assembly 508 to skin of a host. A distal surface 510 of a bottom opening of inner housing 506 may define a bottom surface of applicator 500. In some examples, upon pressing applicator 500 against skin of the host, skin may deform in a substantially convex shape at distal surface 510 such that at least a portion of a surface of skin disposed at the bottom opening of applicator inner housing 506 extends into the bottom opening of inner housing 506 beyond a plane defined by distal surface 510 in a proximal direction.

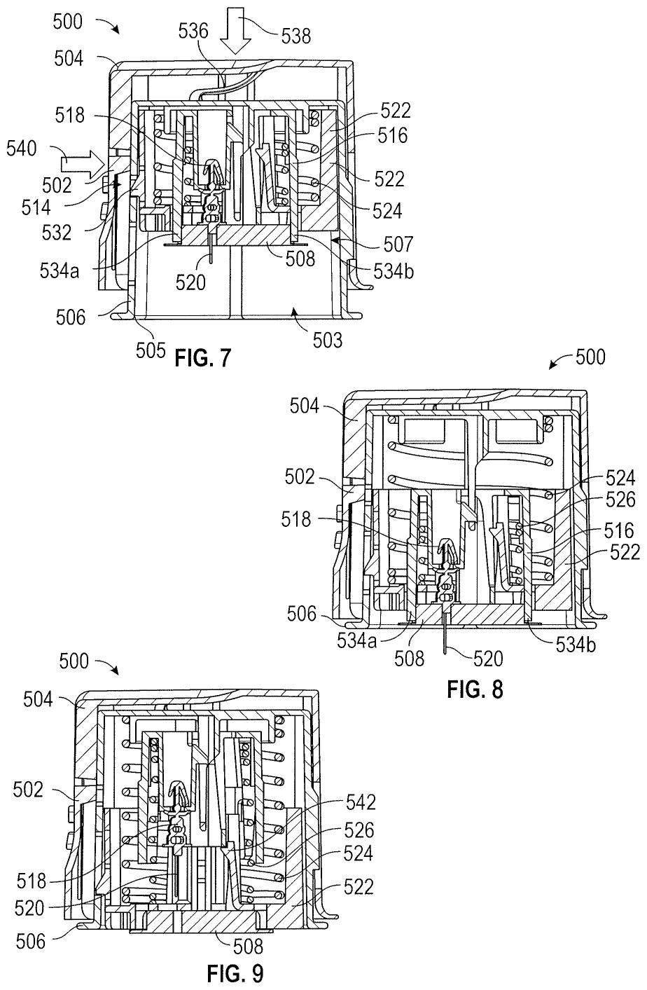

As shown in FIG. 7, the housing 501, and particularly the inner housing 506 may include an internal cavity 503 for retaining the on-skin wearable medical device. The internal cavity 503 may have a distal end portion 505 at the opening for on-skin wearable medical device to be deployed from. A proximal end portion 507 of the internal cavity 503 may include the on-skin wearable medical device coupled to the needle carrier assembly 516.

Referring back to FIG. 6, in some examples, a first barrier layer 512 may be disposed over one or more apertures in inner housing 506, for example, an aperture 514 through which at least a portion of activation element 502 may be configured to extend through during activation of applicator 500. In such examples, a portion of activation element 502 may be configured to pierce or deform first barrier layer 512 upon activation of applicator 500. First barrier layer 512 may comprise a gas permeable material such as Tyvek, or a non-gas permeable material such as metallic foil, polymer film, elastomer, or any other suitable material.

Applicator 500 may further comprise a needle carrier assembly 516, including a needle hub 518 configured to couple an insertion element 520 to needle carrier assembly 516. In some other examples, insertion element 520 may be directly coupled to needle carrier assembly 516. Insertion element 520 is configured to insert sensor of on-skin sensor assembly 508 into skin of the host. In some examples, the insertion element comprises a needle, for example, an open sided-needle, a needle with a deflected-tip, a curved needle, a polymer-coated needle, a hypodermic needle, or any other suitable type of needle or structure. In yet other examples, insertion element 520 may be integrally formed with sensor and may be sufficiently rigid to be inserted partially into skin of the host with minimal or no structural support.

Applicator 500 may further include a holder 522 releasably coupled to needle carrier assembly 516 and configured to guide needle carrier assembly 516 and on-skin sensor assembly 508 while coupled to needle carrier assembly 516, e.g., at least during translation from a proximal position to a distal insertion position. As will be described in more detail below, on-skin sensor assembly 508 may be stripped or released from holder 522 and/or needle carrier assembly 516 once on-skin sensor assembly 508 is disposed on skin of the host. For example, an engagement portion or one or more retention elements may release the on-skin wearable medical device from the applicator housing 501.

Applicator 500 may further comprise an insertion assembly configured to translate insertion element 520, needle hub 518, needle carrier assembly 516, and on-skin sensor assembly 508 from a proximal position, in the distal direction, to a distal insertion position. Such an insertion assembly may include at least one spring for inserting at least a portion of the on-skin wearable device into the skin. The insertion assembly may include a first spring 524. First spring 524 may be a compression spring, or any suitable type of spring, and may have a first end in contact with or coupled to inner applicator housing 506 and a second end in contact with or coupled to holder 522. First spring 524 is configured to, upon activation of the insertion assembly, translate holder 522, needle carrier assembly 516, needle hub 518, insertion element 520 and on-skin sensor assembly 508, in the distal direction to the distal insertion position. Substantially at the distal insertion position, needle carrier assembly 516 may decouple from holder 522 and on-skin sensor assembly 508.

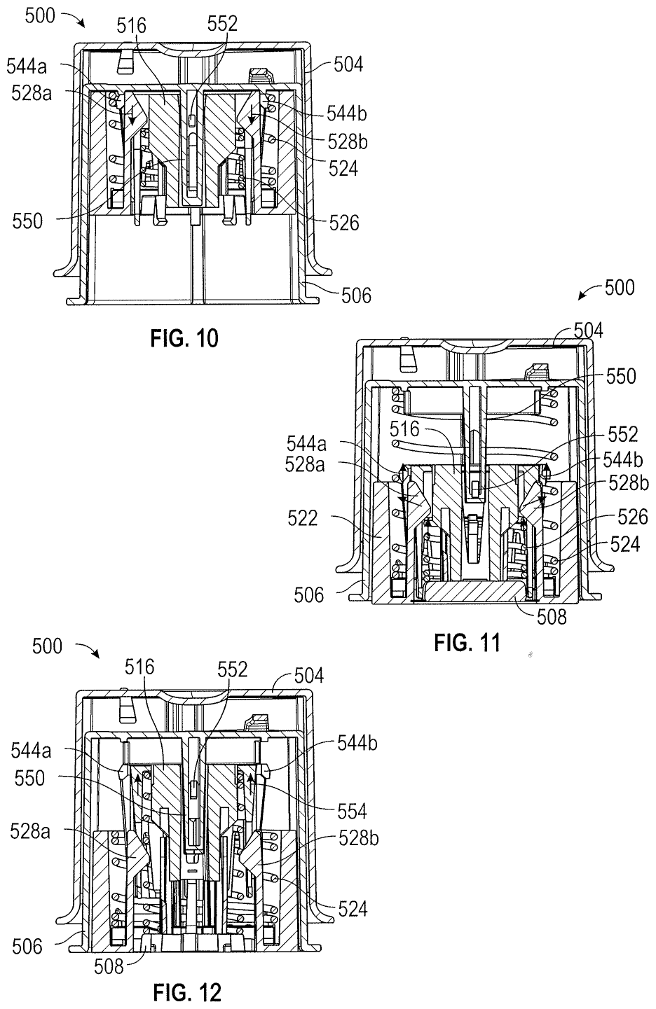

Applicator 500 may further comprise a retraction assembly for retracting the insertion element (e.g., needle) from the skin. The retraction assembly may be configured to translate needle carrier assembly 516, needle hub 518 and insertion element 520, in the proximal direction, from the distal insertion position to a proximal retracted position. In some examples the initial proximal position may be the same as the proximal retracted position. In other examples, the initial proximal position may be different from the proximal retracted position. Such a retraction assembly may include at least one spring. The retraction assembly may include a second spring 526. Second spring 526 may be a compression spring, or any suitable type of spring, and may have a first end contacting or coupled to holder 522 and a second end in contact with or coupled to at least one spring retention element (e.g., 528a, 528b in FIGS. 10-14), at least until retraction. Second spring 526 is configured to translate needle carrier assembly 516, needle hub 518, and insertion element 520 in the proximal direction from the distal insertion position to the proximal retracted position in response to on-skin sensor assembly 508 contacting skin of the host, and/or reaching a limit of travel with a force exceeding a predetermined threshold sufficient to cause first end of second spring 526 to overcome the at least one spring retention element (e.g., 528a, 528b in FIGS. 10-14). In some examples, a stop feature (not shown) may be disposed at a bottom of applicator 500, e.g., on a distal portion of inner housing 506. Such a stop feature may be configured to contact one or more of on-skin sensor assembly 508, needle carrier assembly 516, or holder 522 in the distal insertion position.

In some examples, a second barrier layer 530 may be disposed over the bottom opening of inner housing 506. Second barrier layer 530 may comprise a gas permeable material such as Tyvek, or a non-gas permeable material such as metallic foil, film. In some examples, second barrier layer 530 may be removed by the host prior to use of applicator 500. In examples comprising one or both of first and second barrier layers 512, 530, such layers may provide a sterile environment between applicator 500 and the outside environment and/or may allow ingress and egress of gas such as during sterilization.

A brief description of some aspects of the operation of applicator 500 follows with respect to FIGS. 7-9, which illustrate several cross-sectional views of applicator 500 of FIGS. 5 and 6 during operation, according to some examples. FIGS. 7-9 may correspond to applicator 500 cut along the section line A-A′ shown in FIG. 5, for example.

FIG. 7 illustrates a state of applicator 500 prior to activation. Holder 522 comprises an insertion assembly retention element 532 configured to contact inner housing 506, thereby immobilizing holder 522, needle carrier assembly 516, needle hub 518, insertion element 520 and on-skin sensor assembly 508, in the pre-activated state.

Needle carrier assembly 516 includes an engagement portion of the applicator. The engagement portion may comprises a plurality of wearable retention and/or alignment elements 534a, 534b configured to extend through holder 522 and releasably couple on-skin sensor assembly 508 to holder 522 and/or to needle carrier assembly 516. The engagement portion may have other configurations in examples. Wearable retention elements 534a, 534b may comprise, e.g., arms, deflection element, tabs, detents, snaps or any other features capable of a retaining function. In some examples, wearable retention elements 534a, 534b may extend around rather than through holder 522. Although two wearable retention elements are illustrated, any number of wearable retention elements are contemplated. In some examples, wearable retention element(s) 534a, 534b may comprise snap fits, friction fits, interference features, elastomeric grips and/or adhesives configured to couple on-skin sensor assembly 508 with needle carrier assembly 516 and/or holder 522.

Inner housing 506 may comprise a spring 536 configured to contact outer housing 504 and maintain a predetermined spacing between outer housing 504 and inner housing 506 in the pre-activation orientation of FIG. 7. Spring 536 may be a compression spring, leaf spring, flex arm spring, a piece of foam or rubber, etc. In some other examples, outer housing 504 may comprise spring 536 and spring 536 may be configured to contact inner housing 506, in a reverse fashion from that shown in FIG. 7.

Activation of applicator 500 may include a host pressing applicator 500 against their skin with sufficient force to translate outer housing 504 in a distal direction, as shown by arrow 538, toward and with respect to inner housing 506 until activation element 502 is aligned with aperture 514 of inner housing 506 and insertion assembly retention element 532 of holder 522. Insertion assembly retention element 532 may comprise, e.g., an arm, a deflection element, a tab, a detent, a snap or any other feature capable of a retaining function. Once such an alignment is achieved, a host may initiate (e.g. pushing) activation element 502, as shown by arrow 540, thereby deflecting insertion assembly retention element 532 sufficiently to release holder 522 from inner housing 506. In some other examples, applicator 500 may be configured such that activation element 502 may be activated first, but that actual insertion is not triggered until outer housing 504 is translated sufficiently in the distal direction toward and with respect to inner housing 506. In yet other examples, activation element 502 may be biased toward a center of applicator 500 such that activation element 502 need not be explicitly activated by the host but, instead, activation element 502 may be configured to automatically initiate insertion upon outer housing 504 being translated sufficiently in the distal direction toward and with respect to inner housing 506.

Such configurations provide several benefits. First, translation of outer housing 504 with respect to inner housing 506 before activation provides a measure of drop protection such that if applicator 500 is accidentally dropped, it may not prematurely fire. Second, spring 536 provides a force bias that the host has to affirmatively overcome by pressing applicator 500 into their skin prior to firing, thereby reducing the probability of activating applicator 500 before it is properly positioned. Further, the host may decide to not fire applicator 500 and discontinue pressing applicator 500 against their skin, in which spring 536 will bias against outer housing 504 and allow outer housing 504 to return to its initial state.

Holder 522, needle carrier assembly 516, needle hub 518, insertion element 520, on-skin sensor assembly 508, first spring 524 and second spring 526 are all shown in pre-activation positions in FIG. 7.

FIG. 8 illustrates applicator 500 during insertion of on-skin sensor assembly 508 but before retraction of needle carrier assembly 516. First spring 524 drives holder 522, needle carrier assembly 516, needle hub 518, insertion element 520, and on-skin sensor assembly 508, in the distal direction toward the distal insertion position. FIG. 8 illustrates a position where on-skin sensor assembly 508 is in contact with skin of the host but where holder 522 is not yet fully driven, by first spring 524, into contact with on-skin sensor assembly 508 or skin of the host.

In some examples, masses of each of holder 522, needle carrier assembly 516, needle hub 518, insertion element 520, and on-skin sensor assembly 508 may be specifically designed to reduce or substantially eliminate a tendency of needle carrier assembly 516, needle hub 518, insertion element 520, and on-skin sensor assembly 508 to detach due to inertial forces from holder 522 while being driven in the distal direction during insertion. In some examples, a force exerted by first spring 524 may be selected to be sufficient for proper operation of applicator 500, while not so large as to further exacerbate such above-described inertially triggered detachment. In some examples, a spring (not shown) may be configured to exert a force against a portion of needle carrier assembly 516, for example in a distal direction, sufficient to prevent needle carrier assembly 516 from inertially triggered detaching from holder 522 during insertion.

FIG. 9 illustrates applicator 500 during activation, as needle carrier assembly 516, needle hub 518 and insertion element 520 are retracted in the proximal direction by second spring 526. In FIG. 9, first spring 524 has fully driven on-skin sensor assembly 508 to the skin of the host. In this position, second spring 526 is released from spring retention elements (e.g., 528a, 528b in FIGS. 10-14) and drives needle carrier assembly 516, needle hub 518, and insertion element 520 in the proximal direction from the distal insertion position. Upon needle carrier assembly 516 reaching the proximal retraction position, needle carrier retention element 542 of holder 522 engages with needle carrier assembly 516, thereby maintaining needle carrier assembly 516, needle hub 518 and insertion element 520 in a locked, retracted position limiting access to insertion element 520. Needle carrier retention element 542 may comprise, e.g., an arm, a deflection element, a tab, a detent, a snap or any other feature capable of a retaining function. In this retracted position, needle carrier assembly 516, needle hub 518, and insertion element 520 is prevented from travelling in a distal direction.

A further description of some aspects of the operation of applicator 500 follows with respect to FIGS. 10-12, which illustrate several cross-sectional views of applicator 500 of FIGS. 5 and 6 during operation, according to some examples. FIGS. 10-12 may correspond to applicator 500 cut along the section line B-B′ shown in FIG. 5, for example. For ease of illustration, needle hub 518 and insertion element 520 are not shown in FIGS. 10-12.

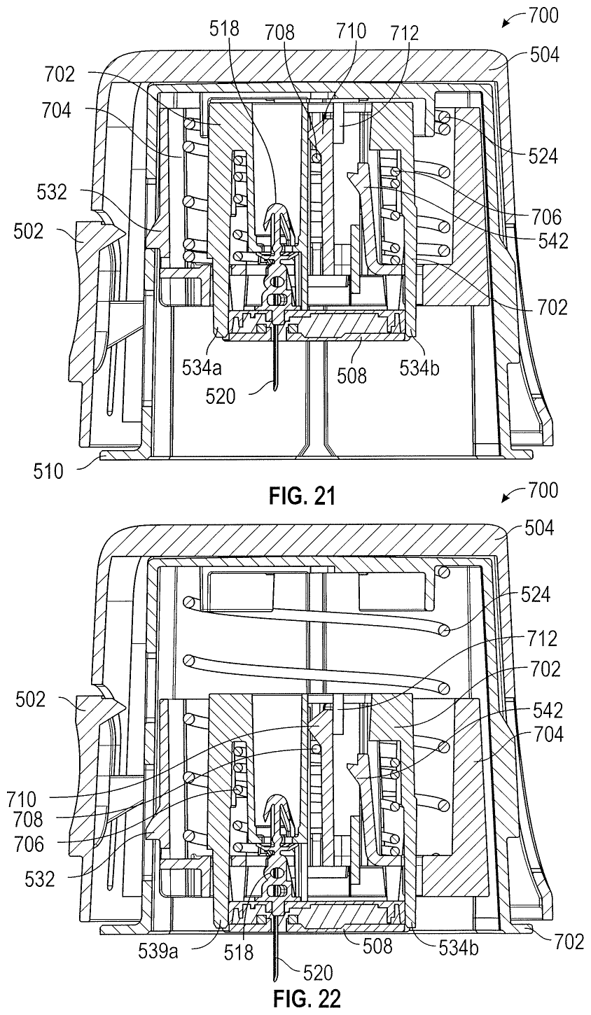

FIG. 10 illustrates a state of applicator 500 prior to activation. For ease of illustration, on-skin sensor assembly 508 is not illustrated in FIG. 10. Holder 522 comprises spring retention elements 528a, 528b configured to contact and retain a first end of second spring 526 in the pre-activated state, e.g., during insertion, while a second end of spring 526 is in contact with needle carrier assembly 516. Spring retention elements 528a, 528b may comprise, e.g., arms, deflection element, tabs, detents, snaps or any other features capable of a retaining function. Although two spring retention elements 528a, 528b are shown, at least one spring retention element is contemplated. In some examples, applicator 500 may include one spring retention element, as shown in FIGS. 21-24. In some examples, applicator 500 may include three spring retention elements. In some examples, applicator 500 may include four spring retention elements. In some examples, spring retention elements 528a, 528b are deflectable arms, rigid arms, deformable features, snaps, catches, or hooks. In some examples, spring retention elements 528a, 528b may be actively deflected by one or more features within applicator 500.

Needle carrier assembly 516 comprises backstop features 544a, 544b, configured to prevent lateral deflection of spring retention elements 528a, 528b in the proximal starting position, e.g., at least during insertion, thereby supporting retention of second spring 526 between spring retention elements 528a, 528b and holder 522 until retraction. Although two backstop features are illustrated, any number of backstop features are contemplated. The number of backstop features may equal the number of spring retention elements.

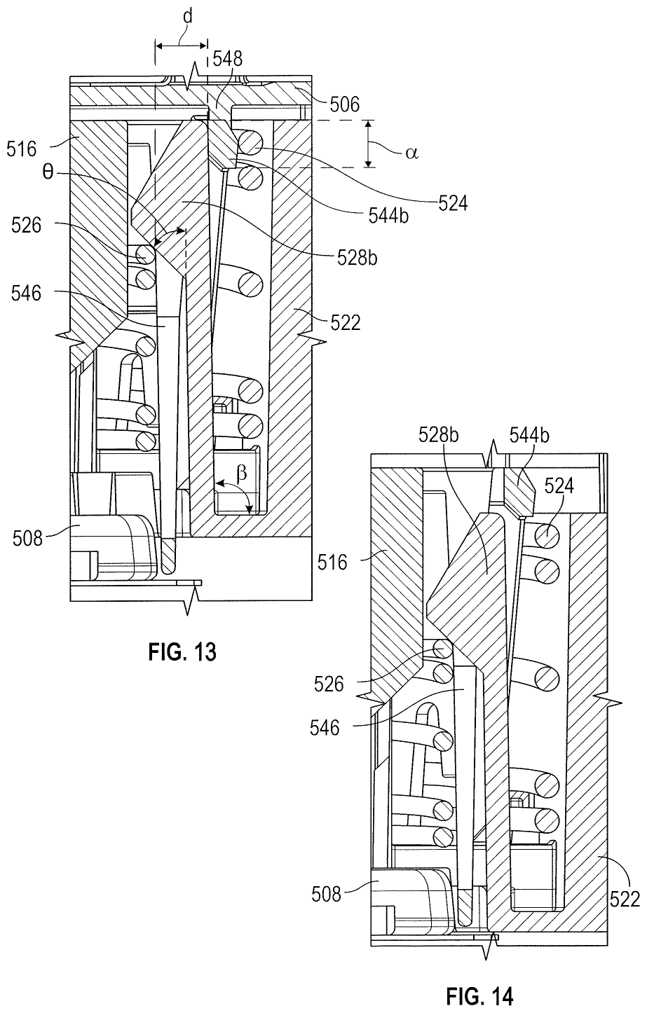

FIG. 13 illustrates a magnified view of spring retention element 528b and backstop feature 544b. In FIG. 13, first spring 524 is driving holder 522, needle carrier assembly 516 and on-skin sensor assembly 508, in the distal direction toward the distal inserted position. Backstop feature 544b is shown engaged to spring retention element 528b, preventing spring retention element 528b from deflecting laterally, thereby preventing second spring 526 from releasing. As shown in FIG. 13, a proximal end of spring retention element 528b may be offset from a distal end of backstop feature 544b by a distance a. In some examples, distance a is the length required for spring retention element 528b to traverse along backstop feature 544b such that spring retention element 528b clears past backstop feature 544b. Backstop feature 544b may feature a ramp to guide spring retention element 528b. A distal end of needle carrier assembly 516 and a distal end of holder 522 may be offset from each other at least the same distance a to allow for spring retention element 528b to traverse distally past backstop feature 544b.

It may be appreciated that the frictional force between corresponding contacting surfaces of backstop feature 544b and spring retention element 528b may at least partly determine an amount of force to release spring retention element 528b from backstop feature 544b. This force may allow for lateral deflection of spring retention element 528b and thus allow the expansion of second spring 526. In some examples, the amount of force is at least 0.1 pounds. In some examples, the amount of force is at least 0.5 pounds. In some examples, the amount of force is at least 1 pound. In some examples, the amount of force is at least 2 pounds. In some examples, the amount of force is at least 3 pounds. In some examples, the amount of force is at least 4 pounds. In some examples, the amount of force is at least 5 pounds.

Although the figure shows backstop feature 544b preventing lateral deflection of spring retention element 528b in a radially outward direction, it is contemplated that an inverse structural relationship can be achieved. For instance, the ramped surface of spring retention element 528b can be reversed to face the opposite direction as shown in FIG. 13. Further, the ramped surface of spring retention element 528b may be biased in a radially inward direction by second spring 526 against backstop feature 544b. In such examples, backstop feature 544b may be located radially inward of spring retention element 528b.

Accordingly, in some examples, materials utilized to form holder 522 and needle carrier assembly 516 may be selected based on a desired amount of force to release spring retention element 528b for lateral deflection. Examples of such materials may include polycarbonate, ABS, PC/ABS, polypropylene, HIPS (High impact polystyrene), polybutylene terephthalate (PBT), polyoxymethylene (POM), acetal, polyacetal, polyformaldehyde, PTFE, high density polyethylene (HDPE), ultra-high-molecular-weight polyethylene (UHMWPE), nylon, polyethylene terephthalate (PET), thermoplastic elastomer (TPE), thermoplastic polyurethane (TPU), TPSiv, cyclo-olefin polymer (COP), cyclo-olefin copolymer (COC), and/or liquid-crystal polymer (LCP).

An angle θ of a portion of spring retention element 528b in contact with second spring 526 may also affect the amount of frictional force to laterally deflect spring retention element 528b and so to release second spring 526. Accordingly, the angle θ may be selected based on a desired amount of force to laterally deflect spring retention element 528b sufficiently to release second spring 526. In some examples, the angle θ is at least 1 degree with respect to a vertical axis of the spring retention element 528b. In some examples, the angle θ is at least 5 degrees. In some examples, the angle θ is at least 10 degrees. In some examples, the angle θ is at least 15 degrees. In some examples, the angle θ is at least 20 degrees. In some examples, the angle θ is about 30 to 45 degrees. In addition, the force profile of second spring 526 may affect a target amount of frictional force to laterally deflect spring retention element 528b. Accordingly, in some examples, the force profile of second spring 526 may be taken into account when selecting one or both of the materials for forming holder 522 and needle carrier assembly 516 and the angle θ of the portion of spring retention element 528b in contact with second spring 526.

An angle ß of spring retention element 528b with respect to a vertical axis may also affect the amount of frictional force to laterally deflect spring retention element 528b and so to release second spring 526. By contacting spring retention element 528b, second spring 526 may exert a force on spring retention element 528b at a distance d from a bottom of spring retention element 528b that causes a torque moment sufficient to induce a lateral deflection of spring retention element 528b.

FIG. 13 further illustrates needle carrier assembly 516 comprising a deflecting element 546 configured to contact spring retention element 528b and maintain spring retention element 528b in a laterally deflected orientation once second spring 526 has initially deflected spring retention element 528b and sufficiently driven needle carrier assembly 516 in the proximal direction, as will be shown in more detail in FIG. 14. Deflecting element 546 may prevent spring retention element 528b from contacting the windings of second spring 526 while second spring 526 is extending, smoothing the operation of applicator 500 and preventing energy released by second spring 526 and designed for driving needle carrier assembly 516 in the proximal direction from being absorbed by undesired contact with spring retention element 528b during the release of second spring 526.

In some examples, the angle θ of the portion of spring retention element 528b in contact with second spring 526 may be substantially 90° (e.g., flat) and deflecting element 546 may have a ramped or angled surface in contact with spring retention element 528b in the position illustrated in FIG. 13. In such examples, deflecting element 546, in addition to the above-described functionality, may be configured to initially deflect spring retention element 528b as first spring 524 drives holder 522 from the position illustrated in FIG. 13 to the position illustrated in FIG. 14.

In some examples, inner housing 506 may comprise a protrusion 548 extending from inner housing 506 in the distal direction. Protrusion 548 may be configured to contact at least one of spring retention elements 528a, 528b and backstop features 544a, 544b in the pre-activation state such that spring retention elements 528a, 528b are prevented from laterally deflecting until holder 522 and needle carrier assembly 516 have translated at least a predetermined minimum distance in the distal direction. Accordingly, protrusion 548 may provide a measure of drop protection such that applicator 500 may not prematurely fire in response to a concussive shock from being dropped before intentional activation.

Turning back to FIG. 10, inner housing 506 may further comprise an engagement element 550 configured to engage with a protrusion 552 of needle carrier assembly 516 upon needle carrier assembly 516 translating in the distal direction beyond a predetermined threshold, thereby preventing needle carrier assembly 516 from translating in the distal direction beyond the predetermined threshold. It is contemplated that this may ensure needle carrier assembly retraction in the event of an air firing or dry firing in which applicator 500 is somehow activated when not held against the skin of the host. In some examples, the predetermined threshold may correspond to the distal end of needle carrier assembly 516 extending beyond a point proximal to the distal end of inner housing 506, to a point substantially in line with the distal end of inner housing 506 or to a point distal of the distal end of inner housing 506. In some examples, engagement element 550 comprises a hook, a U-shaped structure, a loop, a protrusion, or any other structure capable of engaging with protrusion 552 as described above.

FIG. 11 illustrates applicator 500 after activation, at a beginning of a force retraction feature process at or near the distal insertion position where on-skin sensor assembly 508 may be in contact with the skin of the host. First spring 524 has driven holder 522, needle carrier assembly 516, needle hub 518, insertion element, and on-skin sensor assembly 508, in the distal direction toward the distal insertion position. During proper operation, holder 522 and on-skin sensor assembly 508 should be pressing against the skin of the host. However, FIG. 11 may also illustrate a dry fire condition, where applicator 500 is not properly pressed against the skin of the host before triggering applicator 500. Accordingly, upon first spring 524 driving holder 522 and needle carrier assembly 516 in the distal direction beyond the predetermined threshold, engagement element 550 contacts protrusion 552, which prevents needle carrier assembly 516 from traveling further in the distal direction, while holder 522 is driven sufficiently further in the distal direction such that backstop features 544a, 544b of needle carrier assembly 516 no longer contact spring retention elements 528a, 528b in the distal insertion position, thereby releasing the first end of second spring 526 and initiating retraction even when applicator 500 is dry fired. The insertion force provided by first spring 524 may be sufficient to additionally overcome the frictional force between corresponding contacting surfaces of backstop feature 544b and spring retention element 528b.

Turning to FIG. 14, first spring 524 has driven holder 522, needle carrier assembly 516 and on-skin sensor assembly 508 in the distal direction to the skin of the host. As first spring 524 drives holder 522, needle carrier assembly 516 and on-skin sensor assembly 508 against the skin of the host, the skin provides a counter force to the force generated by first spring 524. The skin may oppose the force of first spring 524 and bias against the distal end of on-skin sensor assembly 508. Because the distal end of holder 522 is offset from the distal end of on-skin sensor assembly 508 as shown in FIG. 13, the counter force provided by the skin is transferred to holder 522 as first spring 524 continues to drive holder 522 towards the skin while on-skin sensor assembly 508 is pressed against the skin. The counter force provided by the skin allows spring retention element 528b to displace past backstop feature 544b. Once spring retention element 528b has cleared distance a past backstop feature 544b, second spring 526 can laterally deflect spring retention element 528b, thereby releasing second spring 526, which drives needle carrier assembly 516 in the proximal direction. Alternatively, as described above in connection with FIG. 13, where the angle θ of the portion of spring retention element 528b in contact with second spring 526 is substantially 90° (e.g., flat), the ramped or angled surface of deflecting element 546 in contact with spring retention element 528b deflects spring retention element 528b sufficiently to release second spring 526, which drives needle carrier assembly 516 in the proximal direction.

In some examples, engagement element 550 may engage protrusion 552 even when applicator 500 is pressed against the skin of a user. In such examples, engagement element 550 engages protrusion 552 as first spring 524 drives holder 522, needle carrier assembly 516, and on-skin sensor assembly 508 against the skin of the host. As explained above, engagement element 550 prevents needle carrier assembly 516 from moving distally when engagement element 550 engages protrusion 552. This allows spring retention elements 528a, 528b to separate away from backstop features 544a, 544b and allow for release of second spring 526. The engagement of engagement element 550 and protrusion 552 may add additional force to the counter force provided by the skin, thus increasing the energy needed to overcome the frictional engagement of spring retention elements 528a, 528b and backstop features 544a, 544b. In some instances, the engagement of engagement element 550 and protrusion 552 provides an immediate impulse force that converts at least some of the initial energy of first spring 524 into energy needed to overcome the frictional engagement of spring retention elements 528a, 528b and backstop features 544a, 544b. It is contemplated that such examples may benefit users with soft skin or higher body fat percentage.

Turning back to FIG. 12, which illustrates applicator 500 during activation, needle carrier assembly 516 is retracted in the proximal direction by second spring 526, as indicated by arrow 554. In FIG. 12, with backstop features 544a, 544b no longer immobilizing spring retention elements 528a, 528b, first end of second spring 526 pushes against spring retention elements 528a, 528b with sufficient force to deflect spring retention elements 528a, 528b in the distal insertion position when on-skin sensor assembly 508 is in contact with skin of the host, allowing second spring 526 to clear spring retention elements 528a, 528b and drive needle carrier assembly 516 in the proximal direction, thereby maintaining needle carrier assembly 516, needle hub 518 (see FIGS. 7-9) and insertion element 520 (see FIGS. 7-9) in a locked, retracted position even in the event of a dry fire.

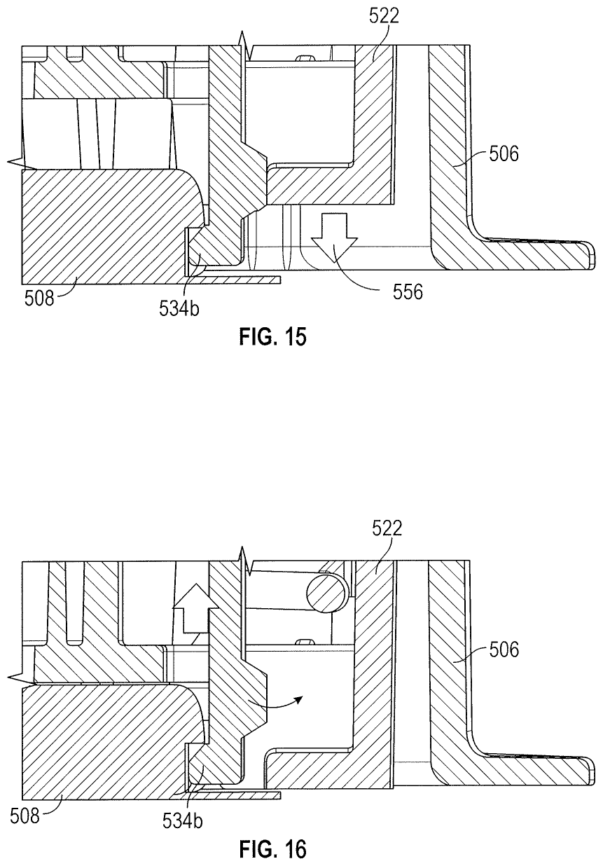

FIGS. 15 and 16 illustrate magnified views of some features of an applicator, such as applicator 500, according to some examples.

In FIG. 15, first spring 524 (see FIGS. 6-12) is driving holder 522, as well as the needle carrier assembly and on-skin sensor assembly 508 in the distal direction, illustrated by arrow 556, toward the distal insertion position. The engagement portion in the form of retention element 534b of the needle carrier assembly is releasably coupled to on-skin sensor assembly 508. As illustrated, during insertion and near the distal inserted position, holder 522 is in contact with spring retention element 534b, preventing spring retention element 534b from deflecting laterally and thereby rigidly securing on-skin sensor assembly 508 to the needle carrier assembly.

In FIG. 16, second spring 526 (see FIGS. 6-12) is driving needle carrier assembly 516 in the proximal direction from the distal insertion position. Because holder 522 has been driven sufficiently in the distal direction, at the distal insertion position, holder 522 is no longer in contact with wearable retention element 534b. Accordingly, wearable retention element 534b is free to deflect laterally, thereby releasing on-skin sensor assembly 508 from wearable retention element 534b and thus from the needle carrier assembly 516. Needle carrier assembly 516 is now driven in the proximal direction by second spring 526, while on-skin sensor assembly 508 is secured to the skin of the host. Moreover, in some examples, because holder 522 is driven to the distal inserted position and substantially held in that position by first spring 524, holder 522 may press against one or both of on-skin sensor assembly 508 or an adhesive patch of on-skin sensor assembly 508, supporting one or both during attachment to the skin of the host.

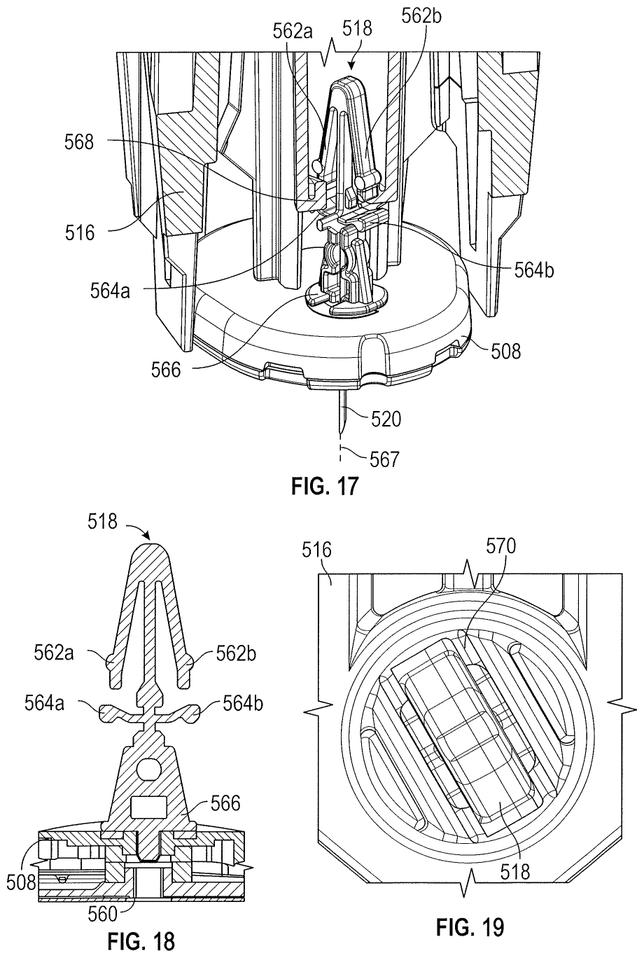

FIG. 17 illustrates a perspective partial cutaway view of needle carrier assembly 516, needle hub 518, and on-skin sensor assembly 508 of applicator 500 of FIGS. 5 and 6, according to some examples. FIG. 18 illustrates a cross-sectional view of needle hub 518 and on-skin sensor assembly 508, according to some examples. FIG. 19 illustrates a top view of a portion of needle carrier assembly 516 and needle hub 518, according to some examples. The following is a description of these features with reference to FIGS. 17-19.

On-skin sensor assembly 508 comprises sensor assembly opening 560. Needle hub 518 is configured to couple insertion element 520 to needle carrier assembly 516 and to substantially maintain a desired orientation of insertion element 520 during insertion of the sensor of on-skin sensor assembly 508 into the skin of the host.

Needle hub 518 comprises a plurality of upper arms 562a, 562b, a plurality of lower arms 564a, 564b, and a base 566. Although two upper arms and two lower arms are illustrated, any number of arms, including a single upper and lower arm, are contemplated. In some examples, upper arms 562a, 562b and lower arms 564a, 564b may be flexible such that, when needle hub 518 is coupled to needle carrier assembly 516, upper arms 562a, 562b and lower arms 564a, 564b secure needle hub 518 in a desired orientation with respect to needle carrier assembly 516. For example, upper arms 562a, 562b may be configured to flex radially inward, such that when disposed through a carrier aperture 568 in needle carrier assembly 516, upper arms 562a, 562b are in contact with an upper surface of needle carrier assembly 516 adjacent to carrier aperture 568 and lower arms 564a, 564b are in contact with a lower surface of needle carrier assembly 516 adjacent to carrier aperture 568. Such an arrangement allows a compliant fit between needle carrier assembly 516 and needle hub 518 where lower arms 564a, 564b deflect to allow upper arms 562a, 562b to expand after clearing surface of carrier aperture 568. The lower arms 564a, 564b can partially or fully relax to bias the needle hub in a distal direction and decrease the clearance between the needle hub and the needle carrier that would otherwise exist with a non-compliant fit. In addition, upper arms 562a, 562b and lower arms 564a, 564b also help to maintain contact between base 566 and a top surface of on-skin sensor assembly 508.

Base 566 comprises an anti-rotation feature. The anti-rotation feature may comprise a key having a shape complementary to at least a portion of sensor assembly opening 560 of on-skin sensor assembly 508 and may be configured to substantially prevent needle hub 518 from rotating about an axis 567 parallel to insertion element 520 with respect to on-skin sensor assembly 508, e.g., to prevent rotation of base 566 within sensor assembly opening 560. In addition, or the alternative, the upper surface of needle carrier assembly 516 adjacent to carrier aperture 568 may comprise a groove 570 configured to accept upper arms 562a, 562b when upper arms 562a, 562b are disposed through carrier aperture 568 in an orientation complementary to an orientation of groove 570, as illustrated in FIG. 19, thereby immobilizing needle hub 518 with respect to needle carrier assembly 516.

In some examples, base 566 further comprises a substantially flat surface configured to mate with a top surface, upper surface, or proximal surface of on-skin sensor assembly 508 and maintain insertion element 520 in a substantially perpendicular orientation to the top surface of on-skin sensor assembly 508, in some cases, when the anti-rotation feature of base 566 is engaged within an opening 560 of on-skin sensor assembly 508.

Based at least upon the above-described features of needle hub 518, on-skin sensor assembly 508, and/or needle carrier assembly 516, base 566 allows easy assembly during manufacture, including but not limited to proper alignment and preassembly of insertion element 520 onto on-skin sensor assembly 508, and/or the ability to easily engage an assembly of needle hub 518, insertion element 520, sensor and on-skin sensor assembly 508 to other portions of assembled applicator 500.

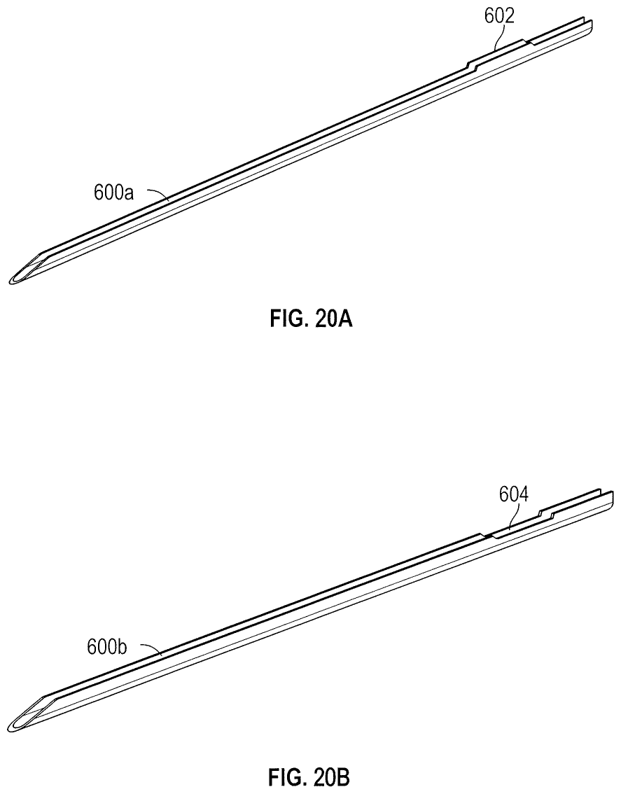

FIGS. 20A and 20B illustrate perspective views of locking features for insertion elements in the form of needles 600a, 600b for use in an applicator for an analyte sensor system, according to some examples. For example, needle 600a of FIG. 20A comprises a locking feature comprising a ridge 602 configured to mate with a complementary-shaped feature within needle hub 518, for example. In the alternative, needle 600b of FIG. 20B comprises a locking feature comprising a groove 604 configured to mate with a complementary-shaped feature within needle hub 518, for example.

In yet another alternative, any insertion element described in this disclosure may comprise a locking feature that heat stakes the selected insertion element to needle hub 518, for example. In yet another alternative, any insertion element described in this disclosure may comprise a locking feature comprising one or more friction-fit or snap-fit elements securing the selected insertion element to needle hub 518, for example. In yet another alternative, any insertion element described in this disclosure may comprise a locking feature comprising complementary clamshell elements on the selected insertion element and needle hub 518, for example, configured to mate with one another. In yet another alternative, any insertion element described in this disclosure may comprise a locking element comprising one or more inserted molded elements configured to couple the selected insertion element to needle hub 518, for example.

During manufacture, applicator 500 may be assembled in stages. For example, and not limitation, if present, first barrier layer 512 may be affixed to inner housing 506. Insertion element 520 may be coupled to needle hub 518, which may then be coupled to on-skin sensor assembly 508. Second spring 526 may be placed into holder 522 or needle carrier assembly 516 and then needle carrier assembly 516 may be disposed into holder 522 and attached to needle hub 518 and to on-skin sensor assembly 508 via wearable retention elements 534a, 534b. First spring 524 may be disposed in holder 522, which may then be installed into inner housing 506. Inner housing 506 may be inserted into and secured to outer housing 504. If present, second barrier layer 530 may be affixed to inner housing 506. If a separate element, activation element 502 may then be disposed into outer housing 504. Any labeling, sterilizing and/or packaging may then be applied to applicator 500.

FIGS. 21-23 illustrate several cross-sectional views, and various features and operating positions, of yet another applicator 700 for an on-skin sensor assembly of an analyte sensor system, according to some examples.

Applicator 700 may include outer applicator housing 504 comprising activation element 502. Outer applicator housing 504 may be configured to translate in a distal direction under force applied by a host of applicator 700, thereby aligning activation element 502 in a position that allows applicator 700 to fire, an alignment illustrated by FIG. 21. As previously described in connection with applicator 500, in some examples, activation element 502 may be disposed in any location, e.g., a top, upper side, lower side, or any other location of applicator 700.

Applicator 700 further comprises inner housing 506, configured to house one or more mechanisms utilized to apply on-skin sensor assembly 508 to skin of a host. Distal surface 510 of a bottom opening of inner housing 506 may define a bottom surface of applicator 700. In some examples, upon pressing applicator 700 against the skin of the host, the skin may deform in a substantially convex shape at distal surface 510 such that at least a portion of a surface of the skin disposed at the bottom opening of applicator housing 506 extends into the bottom opening of inner housing 506, in a proximal direction, beyond a plane defined by distal surface 510.

Although not illustrated in FIGS. 21-23, inner housing 506 may comprise a spring 536 configured to contact outer housing 504 and maintain a predetermined spacing between outer housing 504 and inner housing 506 in the pre-activation orientation (see FIG. 7). Spring 536 may be a compression spring, leaf spring, flex arm spring, a piece of foam or rubber, etc. In some other examples, outer housing 504 may comprise spring 536 and spring 536 may be configured to contact inner housing 506.

Applicator 700 may further comprise a needle carrier assembly 702. Needle carrier assembly 702 comprises an engagement portion, which may be in the form of wearable retention and/or alignment elements 534a, 534b configured to pass through holder 704 and releasably couple on-skin sensor assembly 508 to holder 704 and/or to needle carrier assembly 702. Although two wearable retention and/or alignment elements are illustrated, any number of wearable retention and/or alignment elements are contemplated.

Applicator 700 further comprises needle hub 518 configured to couple insertion element 520 to needle carrier assembly 702. Insertion element 520 is configured to insert sensor of on-skin sensor assembly 508 into skin of the host. In some examples, insertion element 520 comprises a needle, for example, an open sided-needle, a needle with a deflected-tip, a curved needle, a polymer-coated needle, a hypodermic needle, or any other suitable type of needle or structure. In yet other examples, insertion element 520 may be integrally formed with sensor, in which insertion element 520 may be sufficiently rigid to be inserted partially into skin of the host with minimal or no structural support.

Applicator 700 may further include holder 704 releasably coupled to needle carrier assembly 702 and configured to guide on-skin sensor assembly 508 while coupled to needle carrier assembly 702, e.g., at least during translation from a proximal position to a distal insertion position. As previously described in connection with applicator 500, on-skin sensor assembly 508 may be stripped or released from holder 704 and/or needle carrier assembly 702 once on-skin sensor assembly 508 is disposed on the skin of the host.

Applicator 700 may further comprise an insertion assembly configured to translate insertion element 520, needle hub 518, and needle carrier assembly 702 from a proximal position, in the distal direction, to a distal insertion position. Such an insertion assembly may include first spring 524. First spring 524 may be a compression spring, or any suitable type of spring, and may have its first end in contact with or coupled to inner applicator housing 506 and its second end in contact with or coupled to holder 704. First spring 524 is configured to, upon activation of the insertion assembly, translate holder 704, needle carrier assembly 702, needle hub 518, insertion element 520 and on-skin sensor assembly 508, in the distal direction to the distal insertion position. Substantially at the distal insertion position, needle carrier assembly 702 may decouple from holder 704 and on-skin sensor assembly 508.

Applicator 700 may further comprise a retraction assembly configured to translate needle carrier assembly 702, needle hub 518 and insertion element 520, in the proximal direction, from the distal insertion position to a proximal retracted position. In some examples the initial proximal position may be the same as the proximal retracted position. In other examples, the initial proximal position may be different from the proximal retracted position. Such a retraction assembly may include a second spring 706. Second spring 706 may be a compression spring, or any suitable type of spring, and may have a first end contacting or coupled to holder 704 and a second end, comprising a tang 708 (e.g., a spring portion or spring end) disposed substantially along a diameter of second spring 706, in contact with or coupled to a spring retention element 710 of holder 704, at least until retraction. Spring retention element 710 may comprise, e.g., an arm, a deflection element, a tab, a detent, a snap or any other feature capable of a retaining function. Spring retention element 710 may have substantially the same form and function as spring retention elements 528a, 528b of applicator 500 except as described below. Second spring 706 is configured to translate needle carrier assembly 702, needle hub 518, and insertion element 520 in the proximal direction from the distal insertion position to the proximal retracted position. Tang 708 of second spring 706 is released from spring retention element 710 in the distal insertion position when spring retention element 710 is not backed up by backstop element 712 and in response to tang 708 of second spring 706 pushing against spring retention element 710 with a force exceeding a predetermined threshold sufficient to overcome and deflect spring retention element 710.

Needle carrier assembly 702 further comprises a backstop feature 712, configured to prevent lateral motion of spring retention element 710 of holder 704 in at least the proximal pre-activation position, thereby supporting retention of second spring 706 between spring retention element 710 and holder 704 until retraction. In the orientation shown in FIG. 21, second spring 706 is exerting a force against spring retention element 710 but backstop feature 712 prevents lateral deflection of retention element 710.

Holder 704 further comprises needle carrier retention element 542, which may comprise a deflectable arm, rigid arm, deformable feature, snap, catch, or hook. Upon needle carrier assembly 702 reaching the proximal retraction position after activation, needle carrier retention element 542 is configured to engage with needle carrier assembly 702, thereby maintaining needle carrier assembly 702, needle hub 518 and insertion element 520 in a locked, retracted position, limiting access to insertion element 520.

Although not illustrated in FIGS. 21-23, inner housing 506 of applicator 700 may further comprise engagement element 550 and needle carrier assembly 702 may further comprise protrusion 552 and may function substantially as previously described in connection with at least FIGS. 10-12.

Although not illustrated in FIGS. 21-23, inner housing 506 of applicator 700 may further comprise a protrusion extending from inner housing 506 in the distal direction, substantially as previously described protrusion 548. Similar to that previously described in connection with FIG. 13, this protrusion may be configured to contact at least one of spring retention element 710 and backstop feature 712 in the pre-activation state such that spring retention element 710 is prevented from laterally deflecting until holder 704 and needle carrier assembly 702 have translated at least a predetermined minimum distance in the distal direction. Accordingly, the protrusion may provide a measure of drop protection such that applicator 700 may not prematurely fire in response to a concussive shock from being dropped before activation.

Applicator 700 functions substantially similarly to applicator 500 with the exception that instead of utilizing spring retention elements 528a, 528b, which are disposed along an outside of second coil of spring 526 and are configured to contact and retain a coil of second spring 526, applicator 700 utilizes spring retention element 710, which is disposed along an inside of second spring 706 and is configured to contact and retain tang 708 of second spring 706 along a diameter of second spring 706. Disposing spring retention element 710 within and substantially along a center of second spring 706, as opposed to along an outside of second spring 706, further ensures that spring retention element 710 does not contact the coils of second spring 706 as second spring 706 extends during retraction, thereby smoothing the operation of applicator 700. In addition, the arrangement including spring retention element 710, as opposed to spring retention elements 528a, 528b mitigates the risk of, and difficulty ensuring that, multiple spring retention elements trigger or are overcome at substantially the same time.

FIG. 21 illustrates a state of applicator 700 prior to activation, according to some examples. Holder 704, needle carrier assembly 702, needle hub 518, insertion element 520, on-skin sensor assembly 508, first spring 524 and second spring 526 are all shown in pre-activation positions.

Retention element 532 of holder 704 is in contact with inner housing 506, thereby immobilizing holder 704, and therefore also needle carrier assembly 702, needle hub 518, insertion element 520 and on-skin sensor assembly 508, in the pre-activated state.

Backstop feature 712 of needle carrier assembly 702 is in contact with and prevents spring retention element 710 from deflecting laterally, thereby ensuring spring retention element 710 retains tang 708 of second spring 706 in the loaded or pre-activation position shown.

Activation of applicator 700 may include a host pressing applicator 700 against their skin with sufficient force to translate outer housing 504 in a distal direction toward and with respect to inner housing 506 until activation element 502 is aligned with insertion assembly retention element 532 of holder 704, as shown in FIG. 21. Once such an alignment is achieved, a host may initiate activation element 502, thereby deflecting insertion assembly retention element 532 sufficiently to release holder 704 from inner housing 506. In some other examples, applicator 700 may be configured such that activation element 502 may be activated first, but that actual insertion is not triggered until outer housing 504 is translated sufficiently in the distal direction toward and with respect to inner housing 506. In yet other examples, activation element 502 may be biased toward a center of applicator 700 such that activation element 502 need not be explicitly activated by the host but, instead, activation element 502 may be configured to automatically initiate insertion upon outer housing 504 being translated sufficiently in the distal direction toward and with respect to inner housing 506.

FIG. 22 illustrates applicator 700 after activation and during insertion, according to some examples. First spring 524 drives holder 704, and so needle carrier assembly 702, needle hub 518, insertion element 520, and on-skin sensor assembly 508, in the distal direction toward the distal insertion position. FIG. 22 illustrates on-skin sensor assembly 508 in contact with skin of the host but where holder 704 is not yet fully driven, by first spring 524, into contact with on-skin sensor assembly 508 or skin of the host.

In some examples, masses of each of holder 704, needle carrier assembly 702, needle hub 518, insertion element 520, and on-skin sensor assembly 508 may be specifically designed to reduce or substantially eliminate a tendency of needle carrier assembly 702, needle hub 518, insertion element 520, and on-skin sensor assembly 508 to detach from holder 704 while being driven in the distal direction during insertion. In some examples, a force exerted by first spring 524 may further be selected to be sufficient for proper operation of applicator 500, while not so large as to further exacerbate such above-described inertially triggered detachment. In some examples, a spring (not shown) may be configured to exert a force against a portion of needle carrier assembly 702, for example in the distal direction, sufficient to prevent needle carrier assembly 516 from inertially triggered detaching from holder 704 during insertion.

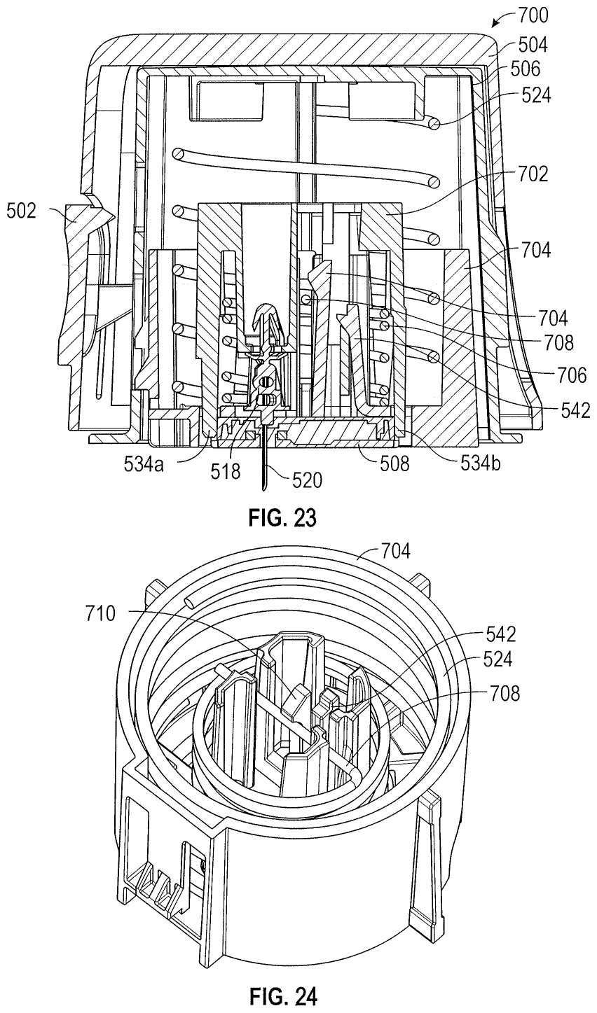

FIG. 23 illustrates applicator 700 after activation and at or near the distal insertion position, according to some examples. First spring 524 has driven holder 704, needle carrier assembly 702 and on-skin sensor assembly 508 in the distal direction to the distal inserted position. Since first spring 524 has driven holder 704 a short distance farther in the distal direction than needle carrier assembly 702, backstop feature 712 is no longer in contact with spring retention element 710, allowing second spring 706 (e.g. tang 708) to laterally deflect spring retention element 710, thereby releasing second spring 706, which drives needle carrier assembly 702 in the proximal direction. Alternatively, similar to that described above in connection with applicator 500 in FIG. 13, where the angle θ of the portion of spring retention element 710 in contact with tang 708 of second spring 526 is substantially 90° (e.g., flat), spring retention element 710 may be biased to automatically deflect sufficiently to release second spring 526 once backstop feature 712 is no longer in contact with spring retention element 710, thereby freeing second spring 706 to drive needle carrier assembly 702 in the proximal direction. Although not shown in FIGS. 21-23, inner housing 506 may further comprise engagement element 550 configured to engage with a protrusion 552 of needle carrier assembly 702, and to function substantially as previously described in connection with at least FIGS. 10-12. In some examples, a stop feature (not shown) may be disposed at a bottom of applicator 700, e.g., on a distal portion of inner housing 506. Such a stop feature may be configured to contact one or more of on-skin sensor assembly 508, needle carrier assembly 702, or holder 704 in the distal insertion position.

Upon release of second spring 706, second spring 706 is configured to drive needle carrier assembly 702, needle hub 518 and insertion element 520, in the proximal direction. Although not shown in FIG. 23, as needle carrier assembly 702 travels to the proximal retracted position, needle carrier retention element 542 may engage with needle carrier assembly 702, thereby retention needle carrier assembly 702, needle hub 518 and insertion element 520, in a locked, retracted position limiting access to insertion element 520.

FIG. 24 illustrates a perspective view of holder 704, first spring 524 and second spring 706 of applicator 700, according to some examples. FIG. 24 illustrates spring retention element 710, retention tang 708 of second spring 706 in an orientation within applicator 700 before retraction.

During manufacture, applicator 700 may be assembled in stages. For example, and not limitation, if present, as previously described in connection with applicator 500, first barrier layer 512 (see FIG. 6) may be affixed to inner housing 506. Insertion element 520 may be coupled to needle hub 518, which may then be coupled to on-skin sensor assembly 508. Second spring may be placed into holder 704 or needle carrier assembly 702 and then needle carrier assembly 702 may be disposed into holder 704 and attached to needle hub 518 and to on-skin sensor assembly via wearable retention elements 534a, 534b. First spring 524 may be disposed in holder 704, which may then be installed into inner housing 506. Inner housing 506 may be inserted into and secured to outer housing 504. If present, as previously described in connection with applicator 500, second barrier layers 530 (see FIG. 6) may be affixed to inner housing 506. If a separate element, activation element 502 may then be disposed into outer housing 504. Any labeling, sterilizing and/or packaging may then be applied to applicator 700.

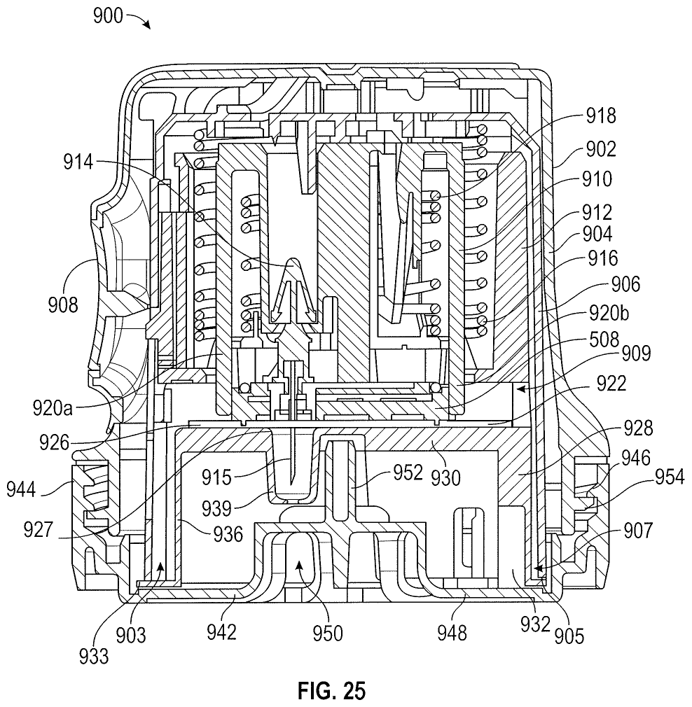

In examples, applicator systems may include a cap and/or a liner removal component. FIG. 25, for example, illustrates an example of an applicator 900 having an applicator housing 902 configured to retain the on-skin wearable medical device, and a deployment mechanism configured to deploy the on-skin wearable medical device to the skin. The applicator housing 902 may be configured similarly as in examples of applicators disclosed herein, including having an outer housing 904 and an inner housing 906 as disclosed in regard to the examples of FIGS. 5-24. The outer housing 904 for example, may be configured similarly as the outer housing 504 and the inner housing may be configured similarly as the inner housing 506. The applicator housing 902 may be configured to be gripped by a user in examples. Various other configurations of applicator housings may be utilized as desired.

The applicator housing 902 may include an internal cavity 903 for retaining the on-skin wearable medical device. The housing 902 may include an opening 905 at an end portion 907 of the internal cavity 903 for the on-skin wearable medical device to be deployed from. The internal cavity 903 may include a proximal end portion 909 that may include the on-skin wearable medical device coupled to a needle carrier assembly.

The deployment mechanism may be configured similarly as other forms of deployment mechanisms disclosed herein. The deployment mechanism may be configured similarly as the deployment mechanisms disclosed in regard to the examples of FIGS. 5-24. For example, the deployment mechanism may include an engagement portion in the form of one or more retention element(s) for retaining the on-skin wearable medical device and releasing the on-skin wearable medical device from the housing 902 to the skin in examples. The deployment mechanism may include an insertion assembly for inserting at least a portion of the on-skin wearable medical device into the skin. The insertion assembly may insert an insertion element (e.g., a needle) into the skin. The deployment mechanism may drive the insertion element to the skin upon the deployment mechanism deploying the on-skin wearable medical device to skin. The deployment mechanism may include a retraction assembly for retracting the insertion element from the skin. Other forms of deployment mechanisms may be utilized in examples as desired.

The applicator 900 may include an activation element 908 that may operate similarly as the activation element 502. The applicator 900 may include a needle carrier assembly 910 that may operate similarly as the needle carrier assembly 516. The applicator 900 may include a holder 912 that may operate similarly as the holder 522. The applicator 900 may include a hub (e.g., a needle hub 914) that may operate similarly as the needle hub 518. The applicator 900 may include an insertion element 915 (e.g., a needle) that may operate similarly as the insertion element 520. The applicator 900 may include springs 916, 918 that may operate similarly as the springs 524, 526 respectively. The applicator 900 may include retention elements 920a, b that may operate similarly as the retention elements 534a, 534b respectively. Additional components of the applicators shown in FIGS. 5-24 may be utilized with the applicator 900. The applicator 900 may operate in a similar manner and provide similar function as the applicators shown in FIGS. 5-24.

The applicator 900 may include a cap 942 that may be positioned at a distal portion of the applicator housing 902 and may cover the distal opening 905 of the internal cavity 903. The cap 942 may include a grip portion 944 on an exterior surface of the cap 942 and an engagement portion 946 on an interior surface of the cap 942. The cap 942 may include a central portion 948 that covers and spans the distal opening 905 of the internal cavity. The cap 942 may comprise an exterior lid for the applicator 900 upon transport and unpackaging of the applicator 900.

The central portion 948 of the cap 942 may include one or more openings 950 that may allow a sterilizing material such as sterilizing gas to pass through, to sterilize internal components of the applicator 900. The central portion 948 may include a central support 952 that may be configured to press against a liner removal component 928 to retain the liner removal component 928 in position. The central support 952 may be configured to rotate upon uncoupling or unscrewing of the cap 942 from the applicator housing 902.

The engagement portion 946 of the cap 942 may comprise threading or another form of engagement portion 946 for engaging a corresponding engagement portion 954 on an exterior surface of the housing 902. The engagement portion 946 may be configured to be rotated relative to the applicator housing 902 to unscrew from the housing 902 and allow for release of the liner removal component 928 from the applicator housing 902.

The applicator 900 may include a liner removal component 928. The liner removal component 928 may be configured to engage a liner 926 positioned on an engaging surface of the patch 922 and remove the liner 926 from the engaging surface of the on-skin wearable medical device upon being withdrawn from the engaging surface of the on-skin wearable medical device. The liner removal component 928 may include an engaging surface 930 for engaging the liner 926. The engaging surface 930 may be a flattened surface that may extend parallel with the liner 926. The engaging surface 930 may include an opening 927 configured to allow the insertion element 915 to pass through. The liner removal component 928 may further include a sheath 939 configured to cover the insertion element 915. The liner removal component 928 may further include a raised portion 936 that may extend from a distal portion 932 of the liner removal component 928. The raised portion 936 may extend axially within the internal cavity 903.

The distal portion 932 of the liner removal component 928 may include a flange 933 for grip by a user to remove the liner removal component 928 from the internal cavity 903 and accordingly remove the liner 926 from the engaging surface of the on-skin wearable medical device. In examples, the flange 933 may be excluded from use.

The liner 926 may be positioned on an engaging surface of the patch in examples. The liner may cover the engaging surface and may protect the engaging surface from damage, deterioration, or other adverse effects. The liner, for example may comprise a sheet of material that covers the engaging surface of the patch. The liner may have a proximal surface contacting the engaging surface of the patch and a distal surface facing opposite the proximal surface. The liner in examples, may be configured to reduce the possibility of an exposed engaging surface from deteriorating or otherwise losing adhesive properties prior to deployment. For example, during a sterilization process using a gas or other sterilizing material, the liner may reduce the possibility of the engaging surface deteriorating. A sterilizing gas may comprise ethylene oxide (EtO) or another form of sterilizing gas as desired. The liner, however, is to be removed from the engaging surface prior to deployment of the on-skin sensor assembly to the skin.

The applicator 900 may be utilized to deploy an on-skin wearable medical device to skin. The on-skin wearable medical device may comprise the on-skin sensor assembly 508 shown in FIG. 6, for example, which may include a housing, an analyte sensor coupled to the housing, an electronics unit, and a patch 922. The on-skin sensor assembly may have forms as shown in FIGS. 2A-4, for example, or other forms as desired.

The cap 942 and the liner removal component 928 may be removed prior to deployment of the on-skin wearable medical device to skin.

Upon activation, an applicator as disclosed herein may insert the analyte sensor into the skin of a host by utilizing an insertion element (such as insertion element 915).

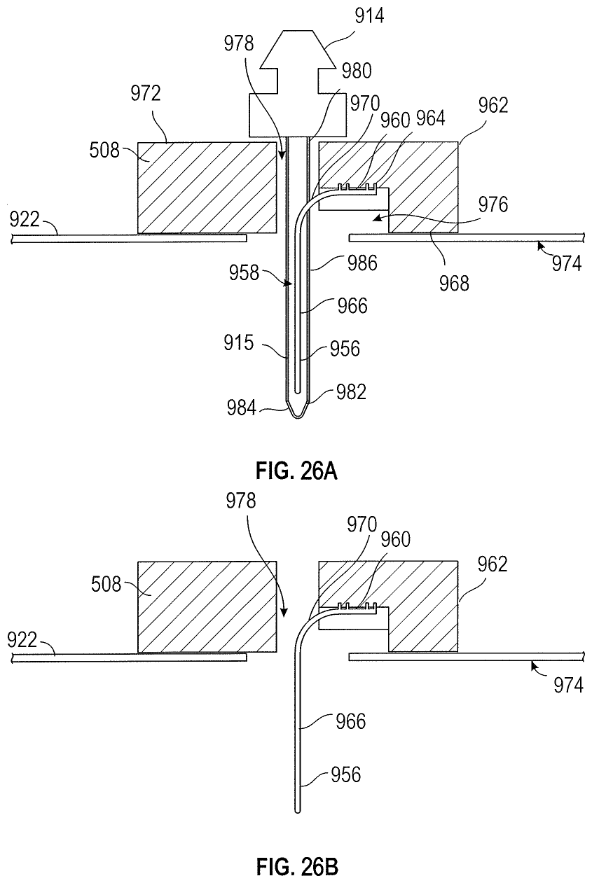

Referring to FIG. 26A, the insertion element 915 may drive the analyte sensor 956 of the on-skin sensor assembly 508 into the host's skin by the analyte sensor 956 extending along a channel 958 of the insertion element 915.

The analyte sensor 956, for example, may include a first portion 960 or contact portion that may be coupled to the housing 962 of the on-skin sensor assembly 508. The first portion 960, for example, may include electrical contacts 964 that may electrically connect to electrical terminals of the on-skin sensor assembly 508 or another component of the on-skin sensor assembly 508. Electrical terminals may be positioned on an interface board or circuit board, or another component of the on-skin sensor assembly 508 as desired. Other methods of coupling between the first portion 960 and the housing 962 may be utilized as desired.

The analyte sensor 956 may include a second portion 966 including a sensing portion that may be configured to be inserted into or through the skin of a host and positioned in or under the skin. The second portion 966, in examples, may extend distally from a distal surface 968 of the housing 962 and may be guided by the insertion element 915 into the skin of the host. The second portion 966 may be straight and may be axially aligned with an opening 978 for the insertion element 915 to pass through, as shown in FIG. 26A.

The analyte sensor 956 may comprise an elongate analyte sensor. The second portion 966 may extend distally to be positioned within the skin layers of the host. In examples, the second portion 966 of the analyte sensor 956 may extend perpendicular with respect to the distal surface 968 of the housing 962. In examples, other angles may be utilized as desired. The second portion 966 may extend perpendicular with respect to the first portion 960 of the analyte sensor 956. In examples, other angles may be utilized as desired.

A bend 970 may angle the second portion 966 of the analyte sensor 956 with respect to the first portion 960 of the analyte sensor 956. The bend 970, for example, may be positioned between the second portion 966 and the first portion 960 and may have a continuous curvature as shown in FIG. 26A or may have another form as desired. The bend 970 may angle the second portion 966 with respect to the first portion 960 at a perpendicular angle or another angle as desired. The bend 970 may be axially aligned with an opening 978 for the insertion element 915 to pass through, as shown in FIG. 26A. Other forms of analyte sensors 956 may be utilized as desired.

The housing 962 of the on-skin sensor assembly 508 may be configured similarly as other forms of housing disclosed herein. The housing 962 may comprise a wearable housing. The housing 962 may be configured to be worn on the skin of the host. The housing 962 may include the distal surface 968, which may be configured to face towards the host's skin. The patch 922 may be positioned on the distal surface 968 of the housing 962. The patch 922 may include the engaging surface 974 for engaging the skin of the host. The engaging surface 974 may comprise an adhesive surface in examples or another form of a surface.

The housing 962 may include a proximal surface 972 facing opposite the distal surface 968. The proximal surface 972 may extend parallel with the distal surface 968 or may have another configuration as desired.

The housing 962 may include a cavity 976 that may receive the first portion 960 of the analyte sensor 956 in examples. The cavity 976 may have a variety of forms as desired. For example, the cavity 976 may be configured to retain an adhesive (which may comprise a liquid adhesive or curable adhesive) that may couple the first portion 960 of the analyte sensor 956 to the housing 962 in examples. The cavity 976 may include one or more dams or other features that may retain the adhesive and may be utilized to electrically isolate portions of the analyte sensor 956 from each other if desired. In examples, the cavity 976 may comprise a recess for the first portion 960 of the analyte sensor 956 to be inserted into, to otherwise couple with the housing 962. In examples, use of a cavity 976 may be excluded and the first portion 960 of the analyte sensor 956 may otherwise couple to the housing 962.

The housing 962 may include an opening 978 for the insertion element 915 to pass through. The opening 978 may extend through the proximal surface 972 of the housing 962 and may extend to the distal surface 968 of the housing 962. The opening 978 may be configured for the insertion element 915 to be retracted proximally through from the skin. The insertion element 915 may be retracted following penetration of the host's skin. In examples, the insertion element 915 may be positioned within the opening 978 upon insertion into the host's skin or may be passed distally relative to the opening 978 upon insertion into the host's skin. In an example as shown in FIG. 26A, the insertion element 915 may be positioned within the opening 978 and may be static relative to the opening 978 upon insertion into the host's skin. For example, as shown in FIGS. 7-8 and 21-23, the insertion element 915 may move distally along with the housing 962 of the on-skin sensor assembly 508 and may remain static relative to the housing 962 upon insertion into the host's skin. Other forms of insertion may be utilized in examples.

The insertion element 915 may include a proximal end portion 980 and a distal end portion 982 comprising a tip 984 of the insertion element 915. The tip 984 may comprise a sharpened tip in examples, and may be configured to puncture the host's skin and be inserted into the host's skin.

The needle hub 914 may be positioned at the proximal end portion 980 of the insertion element 915. The needle hub 914 may be in contact with the proximal surface 972 of the housing 962 or may be spaced from the proximal surface 972 as desired.

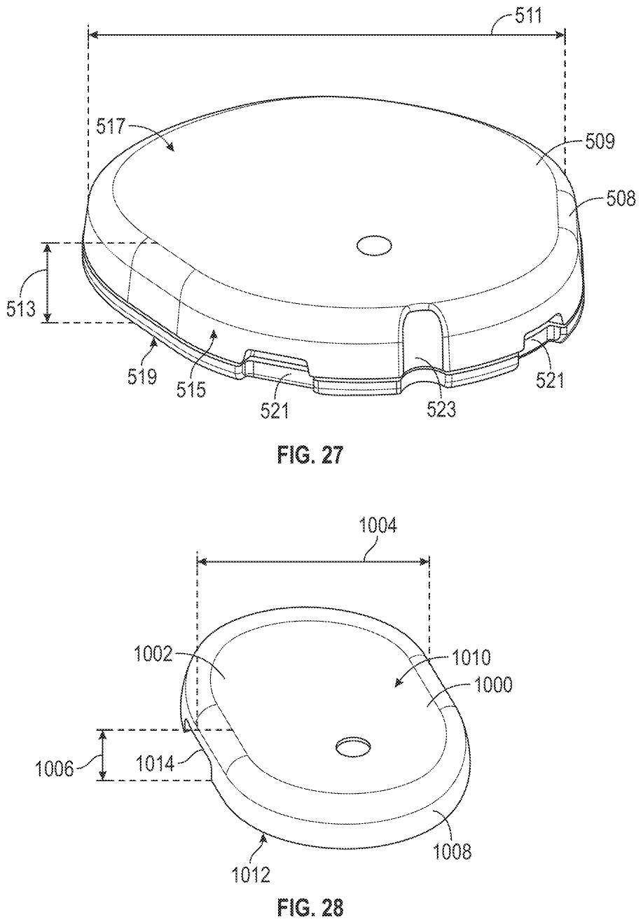

FIG. 27 illustrates a perspective view of the on-skin wearable medical device in the form of on-skin sensor assembly 508. The wearable housing 509 of the on-skin sensor assembly 508 is shown, with the patch 922 (marked in FIG. 25) excluded from view for clarity.

The wearable housing 509 has a diameter 511 and a height 513 (marked in FIG. 27). The wearable housing 509 further may include one or more outer side surfaces 515 having a contour that defines a shape of the wearable housing 509. The wearable housing 509 includes an outer upper, top, or proximal surface 517 having a shape (e.g., a flat shape as shown in FIG. 27). The wearable housing 509 includes an outer lower, bottom, or distal surface 519 having a shape. A distance from the outer upper surface 517 to the outer lower surface 519 of the housing 509 may comprise the height 513 of the housing 509.

The wearable housing 509 may further comprise one or more coupling features 521 that may be configured to engage an engagement portion of the applicator. The coupling features 521 may have a variety of forms, and may comprise recesses as shown in FIG. 27. Other forms of coupling features (e.g., protrusions, adhesives) may be utilized in examples as desired. The coupling features 521 may be positioned on the one or more outer side surfaces 515 of the housing 509 or may have other positions in examples as desired. The coupling features 521 may be configured to engage an engagement portion of an applicator, which may be in the form of wearable retention and/or alignment elements 534a, 534b as shown in FIGS. 15 and 16 for example.

In examples, a stabilization feature 523 may be provided that may stabilize the wearable housing 509 within the applicator. The stabilization feature 523, for example, may comprise a recess in one or more of the outer side surfaces 515 that may mate with a stabilizer of the applicator. The stabilizer may contact the stabilization feature 523 to prevent dislodgement or other movement of the on-skin sensor assembly 508 within the applicator.

The configuration of the on-skin sensor assembly 508 may fit the applicator (e.g., the applicators disclosed herein including applicators 500, 700). For example, the size of the on-skin sensor assembly 508 (e.g., a diameter 511 and/or height 513 of the wearable housing 509) may fit to the applicator and may fit to an engagement portion of the applicator. An engagement portion in the form of wearable retention and/or alignment elements 534a, 534b as shown in FIGS. 15 and 16 for example, may be sized and otherwise configured to engage with the wearable housing 509 of the on-skin sensor assembly 508.

The position and shape of the coupling features 521 may further be configured to engage the engagement portion of the applicator. For example, the coupling features 521 may be positioned, shaped, or otherwise configured for the wearable retention and/or alignment elements 534a, 534b to enter into as shown in FIGS. 15 and 16. The stabilization feature 523 may further be positioned, shaped, or otherwise configured to mate with the stabilizer of the applicator.

The applicator (e.g., the applicators disclosed herein including applicators 500, 700) accordingly may be configured to engage and deploy the on-skin sensor assembly 508 shown in FIG. 27. The engagement portion of the applicator (e.g., wearable retention and/or alignment elements 534a, 534b as shown in FIGS. 15 and 16 for example) may fit the on-skin sensor assembly 508.

Other forms of on-skin wearable medical devices may be utilized in examples however. For example, FIG. 28 illustrates a perspective view of an on-skin wearable medical device having a different configuration than the on-skin wearable medical device shown in FIG. 27. FIG. 28 illustrates an on-skin wearable medical device in the form of an on-skin sensor assembly 1000. The wearable housing 1002 of the on-skin sensor assembly 1000 is illustrated (with a patch excluded from view for clarity). A patch may have a configuration similar to other configurations of patches disclosed herein, or may have other configurations as desired.

The wearable housing 1002 may have a diameter 1004 and a height 1006. The wearable housing 1002 further may include one or more outer side surfaces 1008 having a contour that defines a shape of the wearable housing 1002. The wearable housing 1002 includes an outer upper, top, or proximal surface 1010 having a shape (e.g., a flat shape as shown in FIG. 28). The wearable housing 1002 includes an outer lower, bottom, or distal surface 1012 having a shape. A distance from the outer upper surface 1010 to the outer lower surface 1012 of the housing 1002 may comprise the height 1006 of the housing 1002.

The wearable housing 1002 may further comprise one or more coupling features 1014. The coupling features 1014 may have a variety of forms, and may comprise recesses as shown in FIG. 28. Other forms of coupling features (e.g., protrusions, adhesives) may be utilized in examples as desired. The coupling features 1014 may be positioned on the one or more outer side surfaces 1008 of the housing 1002 or may have other positions in examples as desired.

The configuration of the on-skin sensor assembly 1000 shown in FIG. 28 differs from the configuration of the on-skin sensor assembly 508 shown in FIG. 27. For example, the size of the on-skin sensor assembly 1000 differs from the size of the on-skin sensor assembly 508 shown in FIG. 27. The diameter 1004 of the housing 1002 is smaller than the diameter 511 of the housing 509, for example. The height 1006 of the housing 1002 is smaller than the height 513 of the housing 509, for example. The height and diameter are both shown to differ in FIGS. 27 and 28, yet in examples, one or more of a height or diameter may differ.

The shape of the on-skin sensor assembly 1000 shown in FIG. 28 differs from the shape of the on-skin sensor assembly 508 shown in FIG. 27. The on-skin sensor assembly 508 shown in FIG. 27, for example, has a wider end portion and a narrow end portion, and the on-skin sensor assembly 1000 shown in FIG. 28 has end portions having the same width. The on-skin sensor assembly 1000 may have an oval shape or elliptical shape, and the on-skin sensor assembly 508 of FIG. 27 may have an ovoid shape with one end portion wider than another end portion.

The configuration of the on-skin sensor assembly 1000 shown in FIG. 28 may differ from the configuration of the on-skin sensor assembly 508 shown in FIG. 27 in that the size, position, or other configuration of coupling features may differ. The coupling features 521 shown in FIG. 27, for example, may be positioned at end portions of the wearable housing 509. The coupling features 1014 shown in FIG. 28 may be positioned at a mid-portion of the wearable housing 1002. The configuration of the on-skin sensor assembly 1000 shown in FIG. 28 may differ from the configuration of the on-skin sensor assembly 508 shown in FIG. 27 in other manners as desired.

The on-skin sensor assembly 1000 accordingly may comprise a coupling mismatch with an applicator (e.g., the applicators disclosed herein including applicators 500, 700). An applicator may be configured to fit an on-skin sensor assembly 508 shown in FIG. 27, yet due to the variation in the configuration of the on-skin sensor assembly 1000 shown in FIG. 28, the applicator may not fit the on-skin sensor assembly 1000. For example, referring to FIGS. 15 and 16, the engagement portion in the form of wearable retention and/or alignment elements 534a, 534b may not be configured to engage with the on-skin sensor assembly 1000.

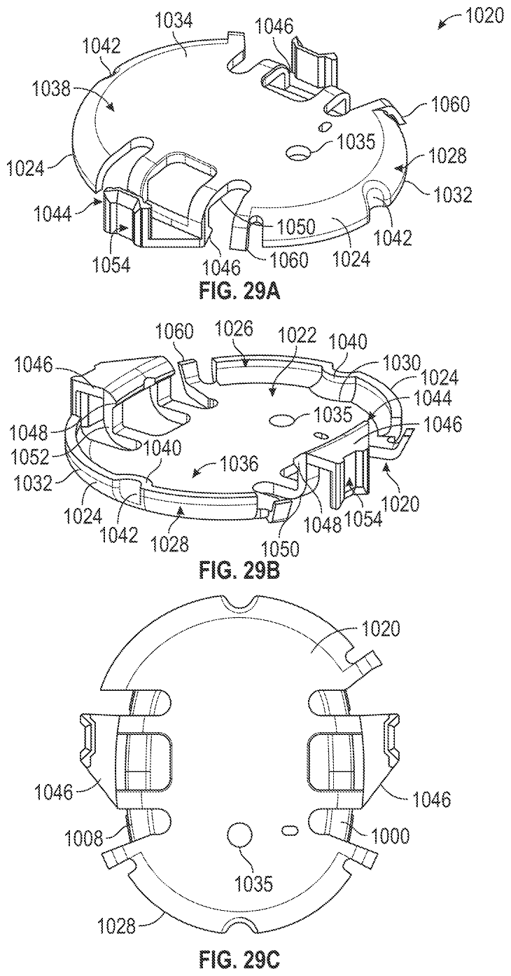

Referring to FIG. 29A, in examples, an adaptor body 1020 may be utilized. The adaptor body 1020 may be configured to interface between at least a portion of an on-skin wearable medical device and an applicator. The adaptor body 1020, for example, may adapt at least the portion of the on-skin wearable medical device to fit an engagement portion of an applicator. For example, referring to FIGS. 27 and 28, the adaptor body 1020 may allow the on-skin sensor assembly 1000 to fit an engagement portion of the applicator that is configured to fit the on-skin sensor assembly 508. The engagement portion may be configured to engage the adaptor body 1020.

In examples, the adaptor body 1020 may be configured to interface in a variety of manners. For example, the adaptor body 1020 may interface between the portion of the on-skin sensor assembly 1000 having a first configuration (as shown in FIG. 28) and a portion of the applicator (e.g., an engagement portion as shown in FIGS. 15 and 16) configured to engage a second configuration of an on-skin sensor assembly 508 (as shown in FIG. 27) that is different than the first configuration. The configurations may differ in one or more of a shape or size, or other configuration (e.g., position or shape of a coupling feature) from each other. The configurations may be of the respective wearable housings 509, 1002 or other portions of the assemblies 508, 1000.

In examples, the on-skin sensor assembly 1000 may be smaller than the on-skin sensor assembly 508. The difference in size may be a difference in size between the housings 509, 1002 of the on-skin sensor assemblies 508, 1000. For example, a smaller diameter or smaller height of the housing 1002 may be provided. The adaptor body 1020 accordingly may comprise a spacer body configured to space the difference in size between the on-skin sensor assemblies 508, 1000. Other forms of interface may be provided in examples.

In examples, the adaptor body 1020 may be configured to adapt an on-skin wearable medical device that may be modified or adjusted from a first configuration to a second configuration. For example, an on-skin wearable medical device may have components or modules added or removed, or other variations in the on-skin wearable medical device that may vary a configuration of the on-skin wearable medical device for engaging with an applicator. The components or modules added or removed may comprise electrical components or modules (e.g., communication components, power sources such as batteries, sensing components, processors, among others) or other configurations of components or modules. The variation in the on-skin wearable medical device may vary a shape, size, or other configuration (e.g., a position or shape of a coupling feature) of the on-skin wearable medical device from the first configuration to the second configuration. The on-skin wearable medical device with the first configuration accordingly may be the same on-skin wearable medical device that has the second configuration. The adaptor body 1020 may be configured to interface between the on-skin wearable device having the second configuration and the applicator (whereas the applicator may be configured to engage the same on-skin wearable device having the first configuration without use of the adaptor body 1020). Other utilizations of the adaptor body 1020 may result.

Referring to FIGS. 29A and 29B, the adaptor body 1020 may include a retention area 1022 for receiving at least a portion of an on-skin wearable medical device. The retention area 1022, for example, may comprise a cavity for receiving at least the portion of the on-skin wearable medical device.

The adaptor body 1020 may include one or more walls that may bound the retention area 1022. The one or more walls may include one or more side walls 1024 that may bound the retention area 1022. The side walls 1024 may be configured to cover corresponding side surfaces 1008 (marked in FIG. 28) of the on-skin sensor assembly 1000.

The side walls 1024 may include an inner surface 1026 (marked in FIG. 29B) and an outer surface 1028. The inner surface 1026 may be configured to face towards the portion of the on-skin wearable medical device that may be positioned within the retention area 1022. The outer surface 1028 may be configured to face opposite the inner surface 1026 and may face radially outward from the on-skin wearable medical device.

The one or more side walls 1024 may have a spacing between the inner surface 1026 and the outer surface 1028 that may be configured to space an outer surface 1008 (marked in FIG. 28) of the on-skin wearable medical device from the outer surface 1028 of the one or more side walls 1024. The outer surface 1008 of the on-skin wearable medical device, for example, may comprise an outer side surface 1008 of the housing 1002. A thickness of the one or more side walls 1024 may form the spacing, or the spacing may be provided in other manners.

The inner surface 1026 may be configured to contact the outer surface 1008 of the on-skin wearable medical device. The inner surface 1026 may be contoured to the shape of the outer surface 1008 of the on-skin wearable medical device to provide a contoured fit to the shape of the outer surface 1008. The outer surface 1028 of the adaptor body 1020 may have a different contour than the inner surface 1026 of the adaptor body 1020 in examples.

For example, the inner surface 1026 may form an inner perimeter 1030 of the adaptor body 1020. The outer surface 1028 may form an outer perimeter 1032 of the adaptor body 1020. The inner perimeter 1030 may have a different contour than the outer perimeter 1032. Such a feature may account for a difference in shape between the outer side surface 1008 of the on-skin sensor assembly 1000 and a shape to which the adaptor body 1020 is adapting the on-skin sensor assembly 1000. For example, if an engagement portion of an applicator is configured to fit a square shape, and the outer side surface 1008 of the on-skin sensor assembly 1000 has an oval shape, then the outer perimeter 1032 may contour to a square shape. The inner perimeter 1030, however, may retain an oval shape to accommodate the shape of the outer side surface 1008 of the on-skin sensor assembly 1000. The inner perimeter 1030 may have a smaller diameter than the outer perimeter 1032 in examples. The engagement portion may be configured to engage at least a portion of one or more walls of the adaptor body. The engagement portion may be configured to engage an outer surface of the adaptor body.

The spacing between the inner surface 1026 and the outer surface 1028 may adapt the on-skin wearable medical device to fit an engagement portion of an applicator. For example, the engagement portion of the applicator may be configured to engage an on-skin sensor assembly having a larger diameter than the on-skin sensor assembly 1000. The spacing may compensate for the difference in diameters. The spacing may alternatively or in combination compensate for a difference in shape between the on-skin sensor assemblies.

The one or more walls may include an upper wall 1034 that may bound the retention area 1022. The upper wall 1034 may be configured to cover a corresponding outer upper, top, or proximal surface 1010 (marked in FIG. 28) of the on-skin wearable medical device. The upper wall 1034 may include an inner surface 1036 (marked in FIG. 29B) and an outer surface 1038. The inner surface 1036 may be configured to face towards the portion of the on-skin wearable medical device that may be positioned within the retention area 1022. The outer surface 1038 may be configured to face opposite the inner surface 1036 and may face radially outward from the on-skin wearable medical device.

The upper wall 1034 may have a spacing between the inner surface 1036 and the outer surface 1038 that may be configured to space an outer upper, top, or proximal surface 1010 (marked in FIG. 28) of the on-skin wearable medical device from the outer surface 1038 of the upper wall 1034. The outer surface 1010 of the on-skin wearable medical device, for example, may comprise an outer upper surface 1010 of the housing 1002. A thickness of the upper wall 1034 may form the spacing, or the spacing may be provided in other manners. The upper wall 1034 may include an opening 1035 that may allow for insertion or retraction of an insertion element, such as a needle.

The inner surface 1036 may be configured to contact the outer surface 1010 of the on-skin wearable medical device. The inner surface 1036 may be contoured to the shape of the outer surface 1010 of the on-skin wearable medical device to provide a contoured fit to the shape of the outer surface 1010. The outer upper surface 1038 of the adaptor body 1020 may have a different contour than the inner surface 1036 of the adaptor body 1020 in examples.

The spacing between the inner surface 1036 and the outer surface 1038 may adapt the on-skin wearable medical device to fit an engagement portion of an applicator. For example, the engagement portion of the applicator may be configured to engage an on-skin sensor assembly having a larger height than the on-skin sensor assembly 1000. The spacing may compensate for the difference in height. The spacing may alternatively or in combination compensate for a difference in shape between the on-skin sensor assemblies.

The side walls 1024 may extend distally from the upper wall 1034 in examples. The side walls 1024 may extend distally from the outer edges or perimeter of the upper wall 1034 in examples. Other configurations may be provided in examples.

One or more stabilizers 1040 may be provided for stabilizing the on-skin wearable medical device within the retention area 1022. The stabilizers may comprise protrusions as shown in FIG. 29B for example. The stabilizers may have other forms in examples. The stabilizers may be configured to contact the on-skin wearable medical device to prevent the on-skin wearable device from moving within the retention area 1022. For example, lateral movement may be resisted. The stabilizers 1040 may comprise a portion of one or more of the side walls 1024, including the inner surface 1026 of the side walls 1024.

Referring to FIGS. 29A and 29B, the outer surface 1028 of the side walls 1024 may include a stabilization feature 1042 that may be configured to stabilize the adaptor body 1020 within an applicator. The stabilization feature 1042 may comprise a recess of the outer surface 1028 of the side walls 1024. The stabilization feature 1042 may have a contour and position similar to the stabilization feature 523 shown in FIG. 27 for example. As such, the stabilization feature 1042 may be utilized to stabilize the adaptor body 1020 in a similar manner as the stabilization feature 523 stabilizes the on-skin sensor assembly 508 within the applicator. Other configurations of stabilization features may be utilized as desired.

The adaptor body 1020 may include a retainer portion 1044 for retaining the on-skin wearable medical device to the adaptor body 1020. The retainer portion 1044 may have a variety of forms. In examples, the retainer portion 1044 may include one or more device couplers 1046 that may couple the on-skin wearable medical device to the adaptor body 1020.

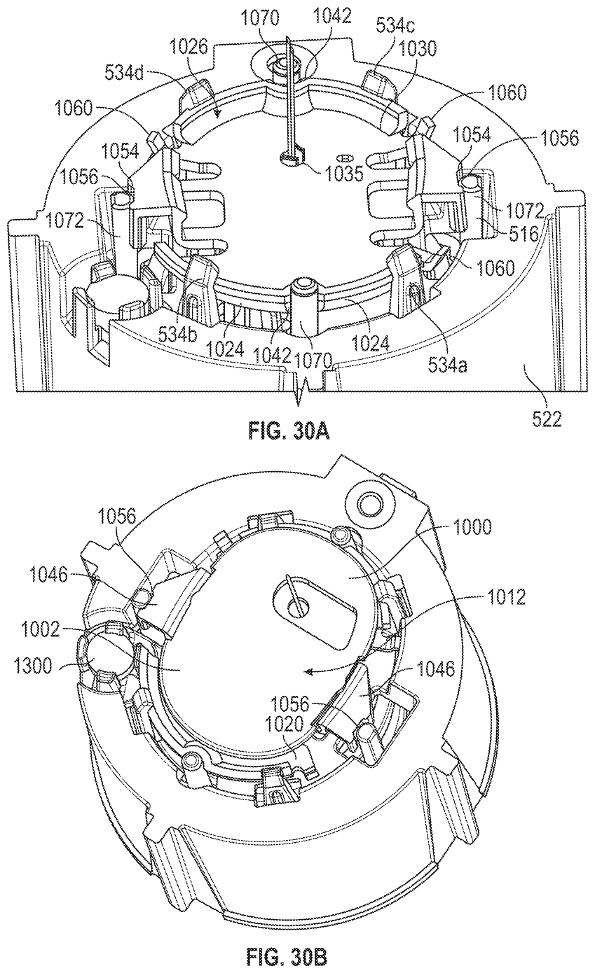

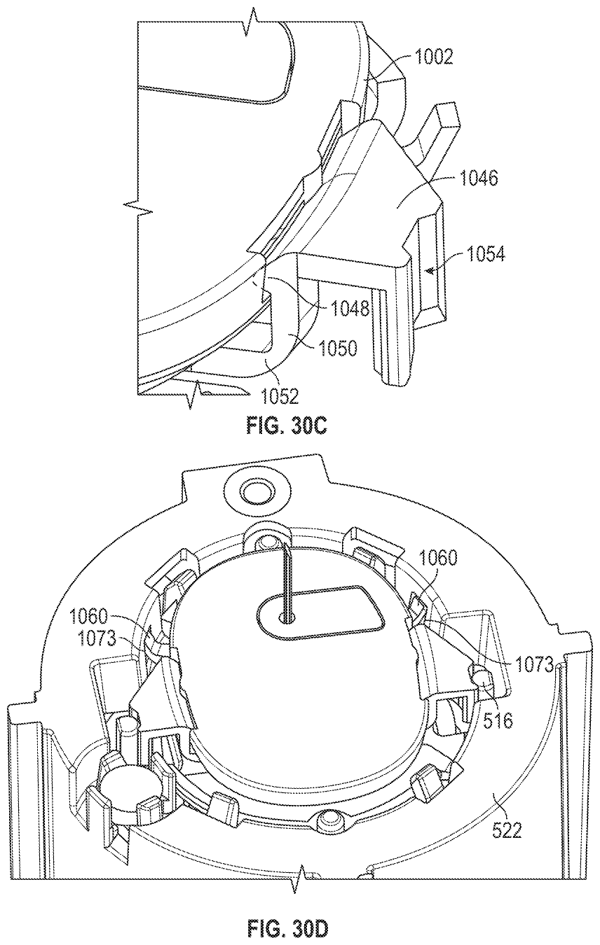

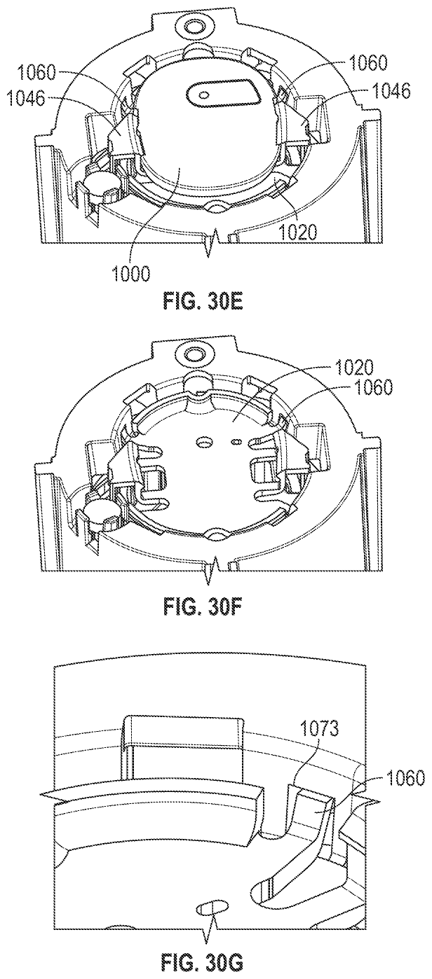

The device couplers 1046 may have a variety of forms in examples. Referring to FIG. 29B, the device couplers 1046 may include protrusions 1048 that may be configured to engage the on-skin sensor assembly 1000. The protrusions 1048 may be configured to engage the coupling features 1014 of the wearable housing 1002 of the on-skin sensor assembly 1000 (as shown in the detail view of FIG. 30C for example). The protrusions 1048 may enter into the coupling features 1014 in the form of recesses in the wearable housing 1002. The device couplers 1046 may have other forms in examples. For example, the device couplers may comprise a protrusion or a recess, or one or more of a protrusion or recess. The device couplers may comprise an adhesive or may have another form.

The device couplers 1046 may be positioned at edge portions of the upper wall 1034, and may comprise portions of the side walls 1024 of the adaptor body 1020 in examples. The device couplers 1046 may have other positions in examples.

The device couplers 1046 may comprise releasable couplers configured to release the on-skin wearable medical device from the adaptor body 1020. The device couplers 1046, for example, may be configured to deflect to release the on-skin wearable medical device from the adaptor body 1020. The deflection may be in a radially outward direction to allow the device couplers 1046 to disengage from the on-skin wearable medical device.

The device couplers 1046 may comprise arms in examples. For example, the arms 1050 may extend from the upper wall 1034 in a distal direction. End portions of the arms 1050 may include the protrusions 1048. The arms 1050 may couple to the upper wall 1034 with a hinge portion 1052. For example, referring to FIG. 30C, the hinge portion 1052 may allow the arm 1050 to deflect radially outward from the wearable housing 1002. The hinge portion 1052 may be configured to pivot about an axis extending parallel with a plane of the adaptor body 1020 (e.g., the plane of the upper wall 1034).

The arms 1050 may bound the retention area 1022 in examples. The arms 1050 may deflect radially outward from the retention area 1022 to allow for release of the on-skin wearable medical device from the retention area 1022 of the adaptor body 1020.

In examples, the adaptor body 1020 may include one or more support portions 1054. The support portions 1054 may be configured to support the device couplers 1046 in a coupled configuration with the on-skin wearable medical device. The support portions 1054 may have a variety of forms and may comprise contact surfaces of the adaptor body 1020. The contact surfaces may be configured to contact with contact surfaces 1056 of the applicator (marked in FIG. 30A) to press the device couplers 1046 towards the on-skin wearable medical device. The contact surfaces 1056 may abut the contact surfaces of the support portions 1054. The device couplers 1046 accordingly may be held in the coupled configuration with the on-skin wearable medical device. The support portions 1054 may have other configurations in examples.

Referring to FIGS. 29A and 29B, applicator couplers 1060 may be utilized for coupling the adaptor body 1020 to at least a portion of the applicator. The applicator couplers 1060 may comprise protrusions that may engage the applicator. The applicator couplers 1060 may have other forms, such as recesses or an adhesive. One or more of a protrusion or recess may be provided in examples. As shown in FIGS. 29A and 29B, the applicator couplers 1060 may protrude radially outward from the outer surface 1028 of the one or more side walls 1024.

FIG. 29C illustrates a top view of the adaptor body 1020 positioned upon the on-skin sensor assembly 1000. The spacing of the outer surface 1028 of the side walls 1024 from the outer side surfaces 1008 of the wearable housing 1002 is visible.

FIG. 30A illustrates the adaptor body 1020 coupled to the applicator. The engagement portion of the applicator, for example, the wearable retention elements 534a-d may engage the side walls 1024 of the adaptor body 1020. Contact surfaces of the side walls 1024 may be positioned in the same position that the coupling features 521 shown in FIG. 27 would engage the wearable retention elements 534a-d. The applicator includes stabilizers 1070 that mate with the stabilization feature 1042 of the adaptor body 1020. The stabilizers 1070 may comprise portions of the needle carrier assembly 516 shown in FIG. 6 for example. The stabilizers 1070 may comprise one or more posts, or may have another form in examples.

The applicator may further include stabilizers 1072 having contact surfaces 1056 for contacting the support portions 1054 of the adaptor body 1020. The stabilizers 1072 may also comprise portions of the needle carrier assembly 516 shown in FIG. 6 for example. The stabilizers 1072 may comprise one or more posts, or may have another form in examples.

The insertion element in the form of a needle may extend through the opening 1035 in the upper surface of the adaptor body 1020.

In a configuration as shown in FIG. 30A, the applicator couplers 1060 may not yet couple to the applicator. This is because the deployment mechanism may be in a distal position, prior to application to the host's skin. The deployment mechanism may be moved proximally upon contact with the host's skin in a deployment procedure.

FIG. 30B illustrates the wearable housing 1002 of the on-skin sensor assembly 1000 coupled to the adaptor body 1020. The device couplers 1046 may couple the wearable housing 1002 to the adaptor body 1020. FIG. 30C illustrates a detail view of the device couplers 1046 coupled to the wearable housing 1002.

FIG. 30D illustrates an exemplary step in the deployment of the on-skin sensor assembly 1000 to the skin of a host. The applicator may be pressed against the surface of the skin, causing the deployment mechanism to retract proximally. The needle carrier assembly 516, for example, may retract proximally relative to the holder 522. Such movement may allow the applicator couplers 1060 to couple to the applicator. The applicator couplers 1060, for example, may enter into recesses or channels 1073 in the holder 522 to couple to the holder 522.

The needle carrier assembly 516 may be retracted in a deployment procedure according to methods disclosed herein. FIG. 30E illustrates a resulting configuration in which needle carrier assembly 516 has been retracted. Accordingly, the engagement portion in the form of the wearable retention elements 534a-d may be released from the adaptor body 1020 in a similar manner as disclosed herein regarding the on-skin sensor assembly 508 (e.g., FIGS. 15 and 16 illustrate an exemplary release). The insertion element in the form of the needle may be retracted. The applicator couplers 1060 may retain the adaptor body 1020 to the applicator.

The stabilizers 1072 (marked in FIG. 30A) having the contact surfaces 1056 for contacting the support portions 1054 of the adaptor body 1020 may be retracted. The support portions 1054 accordingly may be unsupported by the contact surfaces 1056 and the device couplers 1046 may be configured to deflect radially outward. The outward deflection of the device couplers 1046 may allow the on-skin sensor assembly 1000 to release from the adaptor body 1020. The on-skin sensor assembly 1000 may be deployed to the skin of the host with an adhesive or other form of engagement with the skin, and thus may remain deployed to the skin upon removal of the applicator from the skin.

FIG. 30F, for example, illustrates a resulting configuration of a portion of the applicator and the adaptor body 1020 with the on-skin sensor assembly 1000 released from the adaptor body 1020. The adaptor body 1020 may be coupled to the applicator with the applicator couplers 1060 positioned within the recesses or channels 1073 of the applicator (as shown in the detail view of FIG. 30G for example).

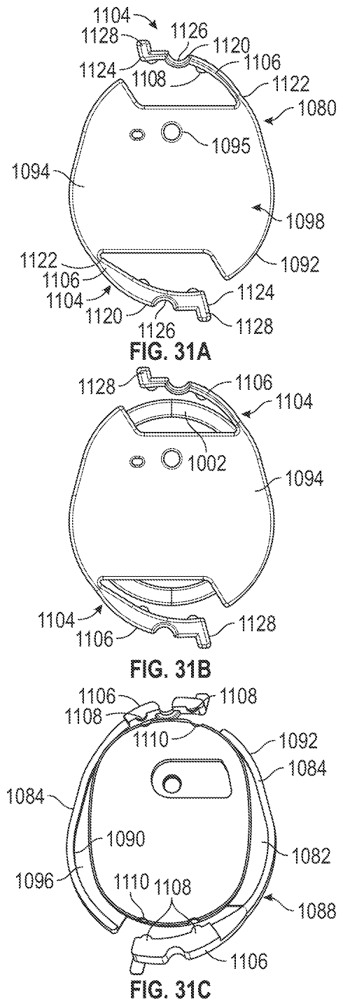

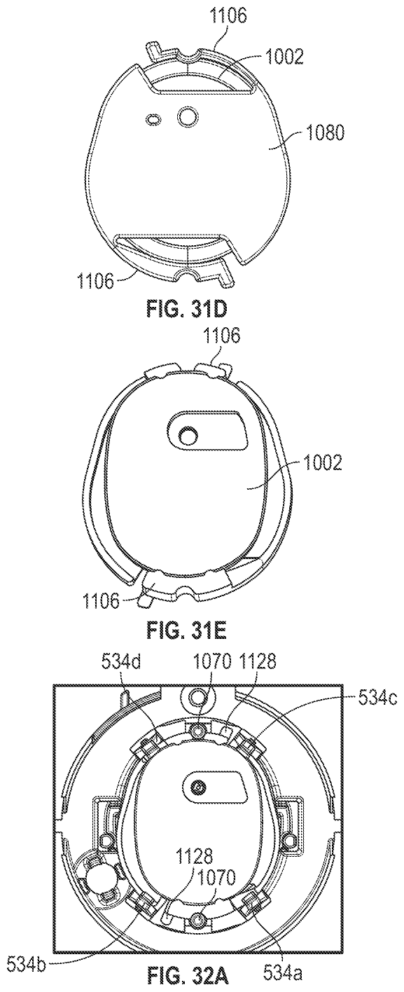

Other configurations of adaptor bodies may be utilized in examples. FIG. 31A illustrates an example of an adaptor body 1080. The adaptor body 1080 may include the features of the adaptor body 1020 unless stated otherwise.

Referring to FIGS. 31A-31C, for example, the adaptor body 1080 may include a retention area 1082 for receiving at least a portion of an on-skin wearable medical device. The retention area 1082, for example, may comprise a cavity for receiving at least the portion of the on-skin wearable medical device.

The adaptor body 1080 may include one or more walls that may bound the retention area 1082. The one or more walls may include one or more side walls 1084 that may bound the retention area 1082. The side walls 1084 may be configured to cover corresponding side surfaces 1008 (marked in FIG. 28) of the on-skin sensor assembly 1000.

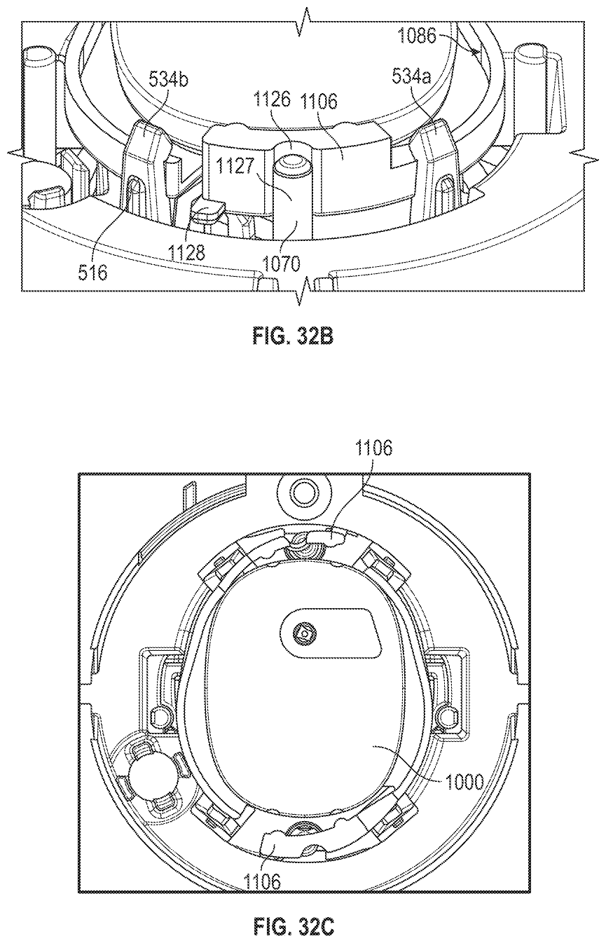

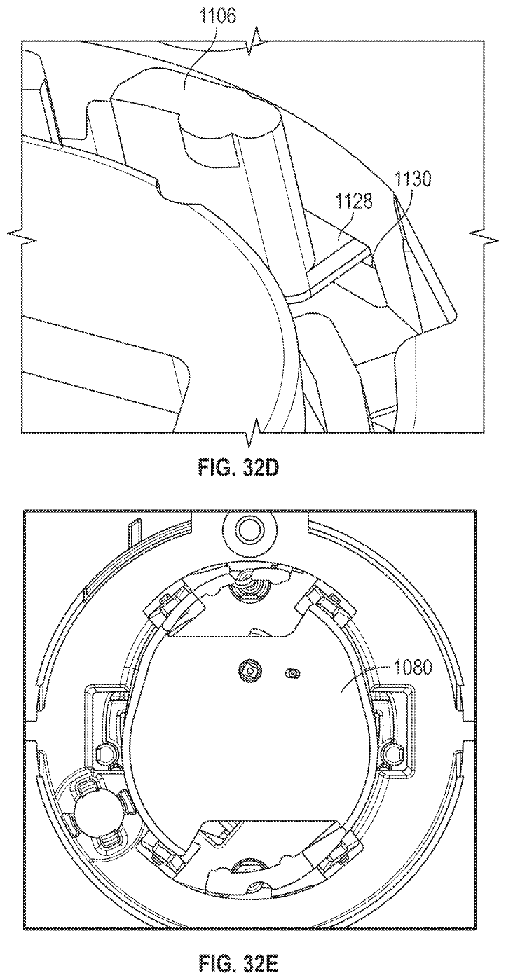

The side walls 1084 may include an inner surface 1086 (marked in FIG. 32B) and an outer surface 1088 (marked in FIG. 31C). The inner surface 1086 may be configured to face towards the portion of the on-skin wearable medical device that may be positioned within the retention area 1082. The outer surface 1088 may be configured to face opposite the inner surface 1086 and may face radially outward from the on-skin wearable medical device.

The one or more side walls 1084 may have a spacing between the inner surface 1086 and the outer surface 1088 that may be configured to space an outer surface 1008 (marked in FIG. 28) of the on-skin wearable medical device from the outer surface 1088 of the one or more side walls 1084. A thickness of the one or more side walls 1084 may form the spacing, or the spacing may be provided in other manners.

The inner surface 1086 may be configured to contact the outer surface 1008 of the on-skin wearable medical device. The inner surface 1086 may be contoured to the shape of the outer surface 1008 of the on-skin wearable medical device to provide a contoured fit to the shape of the outer surface 1008. The outer surface 1088 of the adaptor body 1080 may have a different contour than the inner surface 1086 of the adaptor body 1080 in examples.

For example, the inner surface 1086 may form an inner perimeter 1090 (marked in FIG. 31C) of the adaptor body 1080. The outer surface 1088 may form an outer perimeter 1092 of the adaptor body 1080. The inner perimeter 1090 may have a different contour than the outer perimeter 1092. Such a feature may account for a difference in shape between the outer side surface 1008 of the on-skin sensor assembly 1000 and a shape to which the adaptor body 1080 is adapting the on-skin sensor assembly 1000. The inner perimeter 1090 may have a smaller diameter than the outer perimeter 1092 in examples.

The spacing between the inner surface 1086 and the outer surface 1088 may adapt the on-skin wearable medical device to fit an engagement portion of an applicator. For example, the engagement portion of the applicator may be configured to engage an on-skin sensor assembly having a larger diameter than the on-skin sensor assembly 1000. The spacing may compensate for the difference in diameters. The spacing may alternatively or in combination compensate for a difference in shape between the on-skin sensor assemblies.

The one or more walls may include an upper wall 1094 (marked in FIG. 31A) that may bound the retention area 1082. The upper wall 1094 may be configured to cover a corresponding outer upper, top, or proximal surface 1010 (marked in FIG. 28) of the on-skin wearable medical device. The upper wall 1094 may include an inner surface 1096 (marked in FIG. 31C) and an outer surface 1098. The inner surface 1096 may be configured to face towards the portion of the on-skin wearable medical device that may be positioned within the retention area 1082. The outer surface 1098 may be configured to face opposite the inner surface 1096 and may face radially outward from the on-skin wearable medical device.

The upper wall 1094 may have a spacing between the inner surface 1096 and the outer surface 1098 that may be configured to space an outer upper, top, or proximal surface 1010 (marked in FIG. 28) of the on-skin wearable medical device from the outer surface 1098 of the upper wall 1094. The outer surface 1010 of the on-skin wearable medical device, for example, may comprise an outer upper surface 1010 of the housing 1002. A thickness of the upper wall 1094 may form the spacing, or the spacing may be provided in other manners. The upper wall 1094 may include an opening 1095 that may allow for insertion or retraction of an insertion element, such as a needle.

The inner surface 1096 may be configured to contact the outer surface 1010 of the on-skin wearable medical device. The inner surface 1096 may be contoured to the shape of the outer surface 1010 of the on-skin wearable medical device to provide a contoured fit to the shape of the outer surface 1010. The outer upper surface 1098 of the adaptor body 1020 may have a different contour than the inner surface 1096 of the adaptor body 1020 in examples.

The spacing between the inner surface 1096 and the outer surface 1098 may adapt the on-skin wearable medical device to fit an engagement portion of an applicator. For example, the engagement portion of the applicator may be configured to engage an on-skin sensor assembly having a larger height than the on-skin sensor assembly 1000. The spacing may compensate for the difference in height. The spacing may alternatively or in combination compensate for a difference in shape between the on-skin sensor assemblies.

The side walls 1084 may extend distally from the upper wall 1094 in examples. The side walls 1084 may extend distally from the outer edges or perimeter of the upper wall 1094 in examples. Other configurations may be provided in examples.

The adaptor body 1080 may include a retainer portion 1104 for retaining the on-skin wearable medical device to the adaptor body 1080. The retainer portion 1104 may have a variety of forms. In examples, the retainer portion 1104 may include one or more device couplers 1106 that may couple the on-skin wearable medical device to the adaptor body 1080.

The device couplers 1106 may have a variety of forms in examples. Referring to FIG. 31C, the device couplers 1106 may include protrusions 1108 that may be configured to engage the on-skin sensor assembly 1000. The protrusions 1108 may be configured to engage coupling features 1110 of the wearable housing 1002 of the on-skin sensor assembly 1000 that may have a different configuration than the coupling features shown in FIG. 28 for example. The coupling features 1110, for example, may be positioned at end portions of the wearable housing 1002. Other locations may be utilized as desired. The protrusions 1108 may enter into the coupling features 1110 in the form of recesses in the wearable housing 1002. The device couplers 1106 may have other forms in examples. For example, the device couplers may comprise a protrusion or a recess, or one or more of a protrusion or recess. The device couplers may comprise an adhesive or may have another form.

The device couplers 1106 may comprise arms in examples. For example, the arms 1120 may extend circumferentially about the outer perimeter of the adaptor body 1080. The arms 1120 may extend from a respective hinge portion 1122 circumferentially to a respective end portion 1124 of the arm 1120. Each arm 1120 may extend circumferentially about a side surface of the wearable housing 1002. Each arm 1120 may include the protrusions 1108.

The hinge portion 1122 may allow the arm 1120 to deflect radially outward from the wearable housing 1002. The hinge portion 1122 may be configured to pivot about an axis extending transverse or perpendicular with a plane of the adaptor body 1080 (e.g., the plane of the upper wall 1094).

The arms 1120 may bound the retention area 1082 in examples. The arms 1120 may deflect radially outward from the retention area 1082 to allow for release of the on-skin wearable medical device from the retention area 1082 of the adaptor body 1080.

The device couplers 1106 may be positioned at edge portions of the upper wall 1094, and may comprise portions of the side walls 1084 of the adaptor body 1080 in examples. The device couplers 1106 may have other positions in examples.

The device couplers 1106 may comprise releasable couplers configured to release the on-skin wearable medical device from the adaptor body 1080. The device couplers 1106, for example, may be configured to deflect to release the on-skin wearable medical device from the adaptor body 1080. The deflection may be in a radially outward direction to allow the device couplers 1106 to disengage from the on-skin wearable medical device.

In examples, the adaptor body 1080 may include one or more support portions 1126. The support portions 1126 may be configured to support the device couplers 1106 in a coupled configuration with the on-skin wearable medical device. The support portions 1126 may have a variety of forms and may comprise contact surfaces of the adaptor body 1080. The contact surfaces may be configured to contact with contact surfaces 1127 of the applicator (marked in FIG. 32B) to press the device couplers 1106 towards the on-skin wearable medical device. The support portions 1126 may comprise recesses in the arms 1120 or may have another configuration in examples. The device couplers 1106 accordingly may be held in the coupled configuration with the on-skin wearable medical device. The support portions 1126 may have other configurations in examples.

The support portions 1126 may further comprise a stabilization feature that may be configured to stabilize the adaptor body 1080 within an applicator. The stabilization feature may operate in a similar manner as other forms of stabilization features disclosed herein.

Referring to FIGS. 31A and 31B, applicator couplers 1128 may be utilized for coupling the adaptor body 1080 to at least a portion of the applicator. The applicator couplers 1128 may comprise protrusions that may engage the applicator. The applicator couplers 1128 may have other forms, such as recesses or an adhesive. One or more of a protrusion or recess may be provided in examples. As shown in FIGS. 31A and 31B, the applicator couplers 1128 may protrude radially outward from the outer surface 1088 of the one or more side walls 1084. The applicator couplers 1128 may be positioned on the device couplers 1106 and may be positioned on the end portions 1124 of the arms 1120. Other positions may be utilized in examples.

In examples, the device couplers 1106 may be biased to extend radially outward from the retention area 1082. FIG. 31A, for example, illustrates a top view of the adaptor body 1080 showing the device couplers 1106 in a free state in which the device couplers 1106 extend radially outward (i.e., an opened configuration). FIG. 31B illustrates a top view of the adaptor body 1080 positioned upon the wearable housing 1002. FIG. 31C illustrates a bottom view of the adaptor body 1080 upon the wearable housing 1002, with the device couplers 1106 in an opened configuration.

FIG. 31D illustrates a top view of the adaptor body 1080 positioned upon the wearable housing 1002, with the device couplers 1106 in a closed configuration. FIG. 31E illustrates a bottom view of the adaptor body 1080 positioned upon the wearable housing 1002, with the device couplers 1106 in a closed configuration.

FIG. 32A illustrates the adaptor body 1080 coupled to the applicator. The engagement portion of the applicator, for example, the wearable retention elements 534a-d may engage the side walls 1084 of the adaptor body 1080. Contact surfaces of the side walls 1084 may be positioned in the same position that the coupling features 521 shown in FIG. 27 would engage the wearable retention elements 534a-d. The applicator includes stabilizers 1070 that mate with the support portions 1126 of the adaptor body 1080. The contact between the stabilizers 1070 and the support portions 1126 of the adaptor body 1080 is shown in the perspective view of FIG. 32B. The contact may retain the device couplers 1106 in the closed configuration.

In a configuration as shown in FIGS. 32A and 32B, the applicator couplers 1128 may not yet couple to the applicator. This is because the deployment mechanism may be in a distal position, prior to application to the host's skin. The deployment mechanism may be moved proximally upon contact with the host's skin in a deployment procedure.

Referring to FIG. 32C, the needle carrier assembly 516 may be retracted in a deployment procedure according to methods disclosed herein. FIG. 32C illustrates a resulting configuration in which needle carrier assembly 516 has been retracted. Accordingly, the engagement portion in the form of the wearable retention elements 534a-d may be released from the adaptor body 1080 in a similar manner as disclosed herein regarding the on-skin sensor assembly 508 (e.g., FIGS. 15 and 16 illustrate an exemplary release). The retraction of the needle carrier assembly 516 may retract the insertion element in the form of a needle.

The stabilizers 1070 (marked in FIG. 30A) having the contact surfaces for contacting the support portions 1126 of the adaptor body 1080 may be retracted. The support portions 1126 accordingly may be unsupported by the contact surfaces and the device couplers 1106 may be configured to deflect radially outward. The outward deflection of the device couplers 1106 may allow the on-skin sensor assembly 1000 to release from the adaptor body 1080. The on-skin sensor assembly 1000 may be deployed to the skin of the host with an adhesive or other form of engagement with the skin, and thus may remain deployed to the skin upon removal of the applicator from the skin.

The outward deflection of the device couplers 1106 may further allow the applicator couplers 1128 to couple to recesses or channels 1130 of the applicator (marked in FIG. 32D). The applicator couplers 1128 accordingly may retain the adaptor body 1080 to the applicator.

FIG. 32E, for example, illustrates a resulting configuration of a portion of the applicator and the adaptor body 1080 with the on-skin sensor assembly 1000 released from the adaptor body 1080. The adaptor body 1080 may be coupled to the applicator with the applicator couplers 1128 positioned within the recesses or channels 1130 of the applicator (as shown in the detail view of FIG. 32D for example).

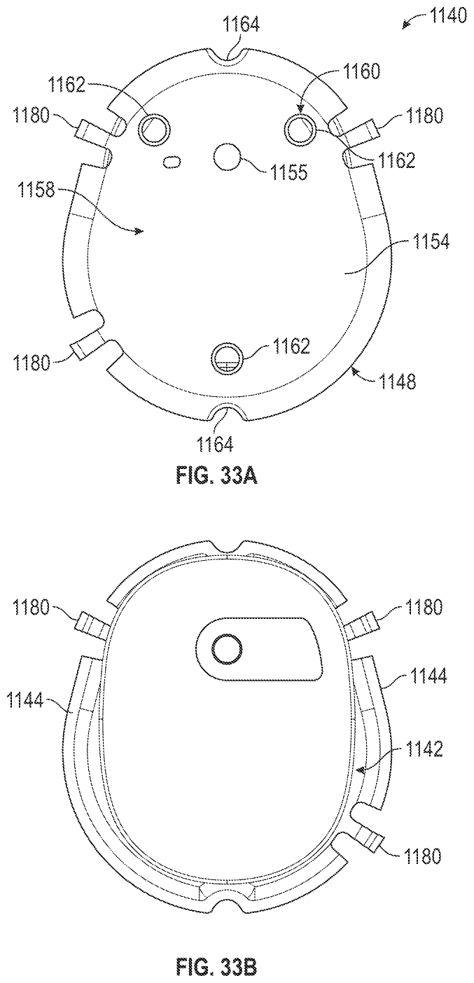

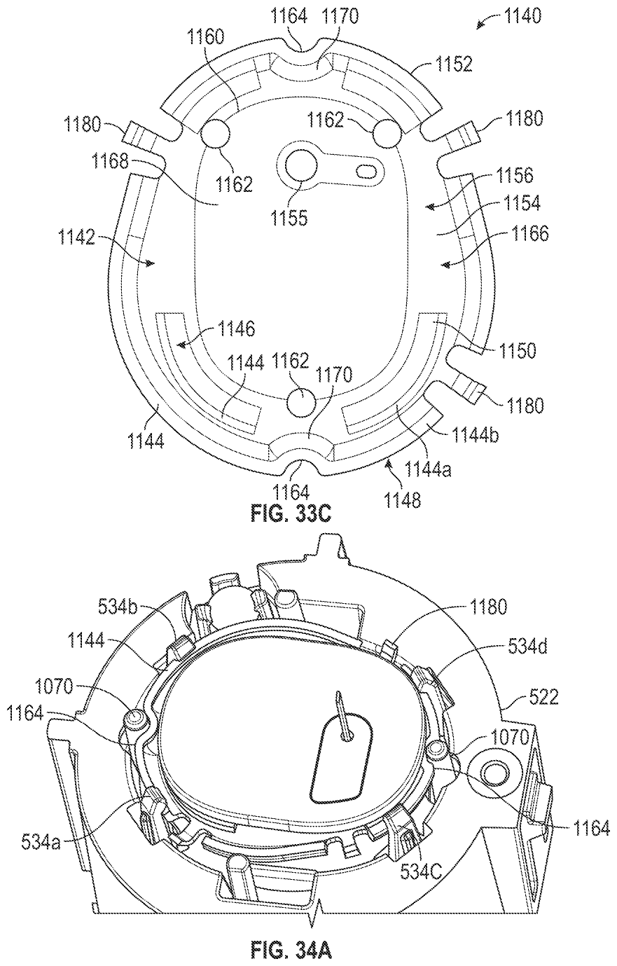

Other configurations of adaptor bodies may be utilized in examples. FIG. 33A illustrates an example of an adaptor body 1140. The adaptor body 1140 may include the features of other forms of adaptor bodies disclosed herein unless stated otherwise.

Referring to FIGS. 33A-33C, the adaptor body 1140 may include a retention area 1142 for receiving at least a portion of an on-skin wearable medical device. The retention area 1142, for example, may comprise a cavity for receiving at least the portion of the on-skin wearable medical device.

The adaptor body 1140 may include one or more walls that may bound the retention area 1142. The one or more walls may include one or more side walls 1144 that may bound the retention area 1142. The side walls 1144 may be configured to cover corresponding side surfaces 1008 (marked in FIG. 28) of the on-skin sensor assembly 1000.

The side walls 1144 may include an inner surface 1146 (marked in FIG. 33C) and an outer surface 1148. The inner surface 1146 may be configured to face towards the portion of the on-skin wearable medical device that may be positioned within the retention area 1142. The outer surface 1148 may be configured to face opposite the inner surface 1146 and may face radially outward from the on-skin wearable medical device.

The one or more side walls 1144 may have a spacing between the inner surface 1146 and the outer surface 1148 that may be configured to space an outer surface 1008 (marked in FIG. 28) of the on-skin wearable medical device from the outer surface 1148 of the one or more side walls 1144. The outer surface 1008 of the on-skin wearable medical device, for example, may comprise an outer side surface 1008 of the housing 1002. The spacing may be provided by the distance from an inner side wall 1144a (as shown in FIG. 33C) relative to an outer side wall 1144b.

The inner surface 1146 may be configured to contact the outer surface 1008 of the on-skin wearable medical device. The inner surface 1146 may be contoured to the shape of the outer surface 1008 of the on-skin wearable medical device to provide a contoured fit to the shape of the outer surface 1008. The outer surface 1148 of the adaptor body 1140 may have a different contour than the inner surface 1146 of the adaptor body 1140 in examples.

For example, the inner surface 1146 may form an inner perimeter 1150 of the adaptor body 1140. The outer surface 1148 may form an outer perimeter 1152 of the adaptor body 1140. The inner perimeter 1150 may have a different contour than the outer perimeter 1152. Such a feature may account for a difference in shape between the outer side surface 1008 of the on-skin sensor assembly 1000 and a shape to which the adaptor body 1140 is adapting the on-skin sensor assembly 1000. The inner perimeter 1150 may have a smaller diameter than the outer perimeter 1152 in examples.

The spacing between the inner surface 1146 and the outer surface 1148 may adapt the on-skin wearable medical device to fit an engagement portion of an applicator. For example, the engagement portion of the applicator may be configured to engage an on-skin sensor assembly having a larger diameter than the on-skin sensor assembly 1000. The spacing may compensate for the difference in diameters. The spacing may alternatively or in combination compensate for a difference in shape between the on-skin sensor assemblies.

The one or more walls may include an upper wall 1154 that may bound the retention area 1142. The upper wall 1154 may be configured to cover a corresponding outer upper, top, or proximal surface 1010 (marked in FIG. 28) of the on-skin wearable medical device. The upper wall 1154 may include an inner surface 1156 (marked in FIG. 33C) and an outer surface 1158 (marked in FIG. 33A). The inner surface 1156 may be configured to face towards the portion of the on-skin wearable medical device that may be positioned within the retention area 1142. The outer surface 1158 may be configured to face opposite the inner surface 1156 and may face radially outward from the on-skin wearable medical device.

The upper wall 1154 may have a spacing between the inner surface 1156 and the outer surface 1158 that may be configured to space an outer upper, top, or proximal surface 1010 (marked in FIG. 28) of the on-skin wearable medical device from the outer surface 1158 of the upper wall 1154. The outer surface 1010 of the on-skin wearable medical device, for example, may comprise an outer upper surface 1010 of the housing 1002. A thickness of the upper wall 1154 may form the spacing, or the spacing may be provided in other manners. The upper wall 1154 may include an opening 1155 that may allow for insertion or retraction of an insertion element, such as a needle.

The inner surface 1156 may be configured to contact the outer surface 1010 of the on-skin wearable medical device. The inner surface 1156 may be contoured to the shape of the outer surface 1010 of the on-skin wearable medical device to provide a contoured fit to the shape of the outer surface 1010. The outer upper surface 1158 of the adaptor body 1140 may have a different contour than the inner surface 1156 of the adaptor body 1140 in examples.

The spacing between the inner surface 1156 and the outer surface 1158 may adapt the on-skin wearable medical device to fit an engagement portion of an applicator. For example, the engagement portion of the applicator may be configured to engage an on-skin sensor assembly having a larger height than the on-skin sensor assembly 1000. The spacing may compensate for the difference in height. The spacing may alternatively or in combination compensate for a difference in shape between the on-skin sensor assemblies.

The side walls 1144 may extend distally from the upper wall 1154 in examples. The side walls 1144 may extend distally from the outer edges or perimeter of the upper wall 1154 in examples. Other configurations may be provided in examples.

In examples, the adaptor body 1140 may include an ejection portion 1160 that may be configured to allow the on-skin wearable medical device to eject from the adaptor body 1140. The ejection portion 1160 may comprise one or more openings 1162 in the upper wall 1154 of the adaptor body 1140. The openings 1162 may be configured to allow an ejection assembly of the applicator to pass through, to contact the on-skin wearable device and cause it to eject from the adaptor body 1140. The ejection assembly, for example, may comprise one or more protrusions configured to pass through the openings 1162.

One or more stabilizers 1170 (marked in FIG. 33C) may be provided for stabilizing the on-skin wearable medical device within the retention area 1142. The stabilizers may comprise protrusions as shown in FIG. 33C for example. The stabilizers may have other forms in examples. The stabilizers may be configured to contact the on-skin wearable medical device to prevent the on-skin wearable device from moving within the retention area 1142. For example, lateral movement may be resisted. The stabilizers 1170 may comprise a portion of one or more of the side walls 1144, including the inner surface 1146 of the side walls 1144.

Referring to FIGS. 33A and 33B, the outer surface 1148 of the side walls 1144 may include a stabilization feature 1164 that may be configured to stabilize the adaptor body 1140 within an applicator. The stabilization feature 1164 may comprise a recess of the outer surface 1148 of the side walls 1144. The stabilization feature 1164 may have a contour and position similar to the stabilization feature 523 shown in FIG. 27 for example. As such, the stabilization feature 1164 may be utilized to stabilize the adaptor body 1140 in a similar manner as the stabilization feature 523 stabilizes the on-skin sensor assembly 508 within the applicator. Other configurations of stabilization features may be utilized as desired.

The adaptor body 1140 may include a retainer portion 1166 for retaining the on-skin wearable medical device to the adaptor body 1140. The retainer portion 1166 may have a variety of forms. In examples, the retainer portion 1166 may include one or more device couplers 1168 that may couple the on-skin wearable medical device to the adaptor body 1140.

Referring to FIG. 33C, a device coupler 1168 may comprise an adhesive that may couple the on-skin wearable medical device to the adaptor body 1140. The adhesive may comprise an adhesive layer comprising the inner surface 1156 of the upper wall 1154. The adhesive may comprise a releasable coupler in the form of a releasable adhesive configured to release the on-skin wearable medical device from the adaptor body 1140.

Referring to FIGS. 33A and 33B, applicator couplers 1180 may be utilized for coupling the adaptor body 1140 to at least a portion of the applicator. The applicator couplers 1180 may comprise protrusions that may engage the applicator. The applicator couplers 1180 may be configured similarly as the applicator couplers 1060 discussed in regard to FIGS. 29A and 29B.

FIG. 33A illustrates a top view of the adaptor body 1140 positioned upon the on-skin sensor assembly 1000. The spacing of the outer surface 1148 of the side walls 1144 from the outer side surfaces 1008 of the wearable housing 1002 is visible.

FIG. 33B illustrates a bottom view of the adaptor body 1140 positioned upon the on-skin sensor assembly 1000. FIG. 33C illustrates a bottom view of the adaptor body 1140 without the on-skin sensor assembly 1000 present.

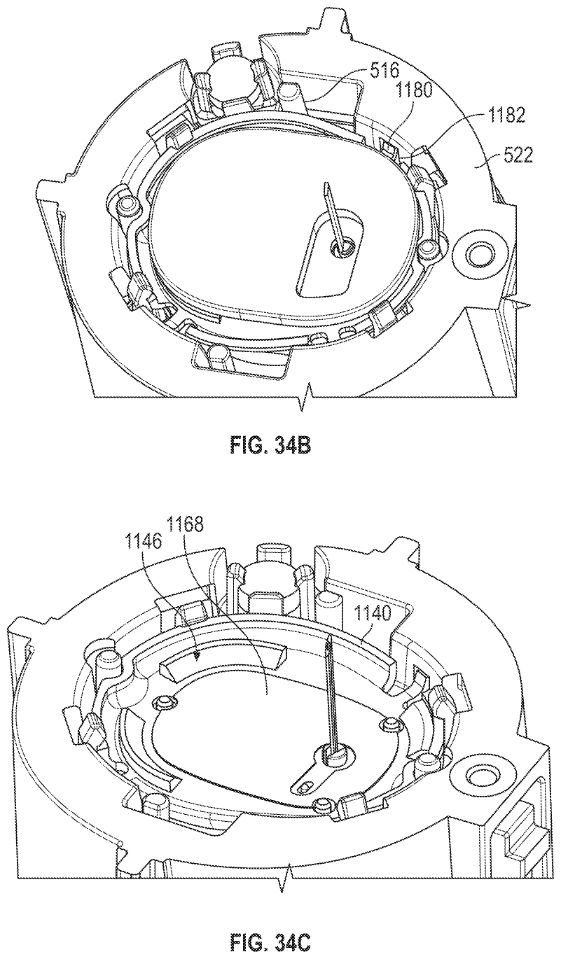

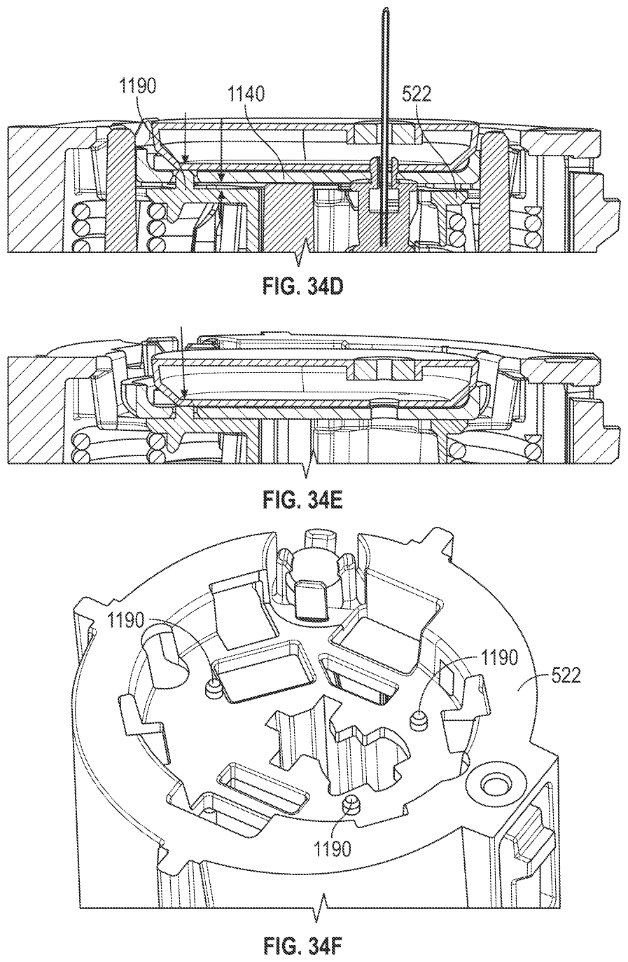

FIG. 34A illustrates the adaptor body 1140 coupled to the applicator. The engagement portion of the applicator, for example, the wearable retention elements 534a-d may engage the side walls 1144 of the adaptor body 1140. Contact surfaces of the side walls 1144 may be positioned in the same position that the coupling features 521 shown in FIG. 27 would engage the wearable retention elements 534a-d. The applicator includes stabilizers 1070 that mate with the stabilization feature 1164 of the adaptor body 1140. The stabilizers 1070 may comprise portions of the needle carrier assembly 516 shown in FIG. 6 for example.

In a configuration as shown in FIG. 34A, the applicator couplers 1180 may not yet couple to the applicator. This is because the deployment mechanism may be in a distal position, prior to application to the host's skin. The deployment mechanism may be moved proximally upon contact with the host's skin in a deployment procedure.

FIG. 34B illustrates an exemplary step in the deployment of the on-skin sensor assembly 1000 to the skin of a host. The applicator may be pressed against the surface of the skin, causing the deployment mechanism to retract proximally. The needle carrier assembly 516, for example, may retract proximally relative to the holder 522. Such movement may allow the applicator couplers 1180 to couple to the applicator. The applicator couplers 1180, for example, may enter into recesses or channels 1182 in the holder 522 to couple to the holder 522. FIG. 34C illustrates a configuration without the on-skin sensor assembly 1000 shown for clarity. The applicator couplers 1180 may retain the adaptor body 1140 to the applicator following release of the on-skin wearable medical device from the adaptor body 1140.

Upon the on-skin sensor assembly 1000 being deployed to the skin of the host, the holder 522 may be advanced distally according to methods disclosed herein. The ejection assembly of the applicator, for example, may operate upon the holder 522 being advanced distally relative to the adaptor body 1140.

FIG. 34D, for example, illustrates the ejection assembly including an ejection actuator in the form of protrusions 1190. The ejection actuator may be configured to eject the on-skin wearable medical device from the adaptor body 1140. The protrusions 1190 of the ejection actuator may extend through the openings of the adaptor body 1140. The protrusions 1190 may advance distally to cause the on-skin sensor assembly 1000 to decouple from the device coupler 1168 by being pressed distally by the protrusions 1190. FIG. 34F, for example, illustrates a distal perspective view of the holder 522 without the adaptor body 1140 present. The protrusions 1190 extend distally from a surface of the holder 522.

The ejection assembly may be pressed distally with the needle carrier assembly 516 withdrawn, as shown in FIG. 34E for example. The on-skin sensor assembly 1000 may release from the adaptor body 1140. The adaptor body 1140 may be coupled to the applicator with the applicator couplers 1180 positioned within the recesses or channels 1182 of the applicator.

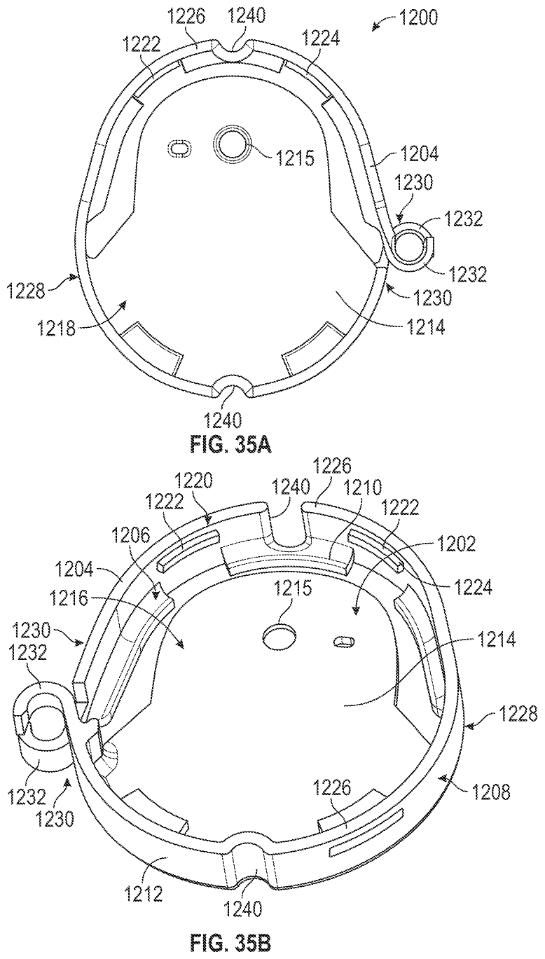

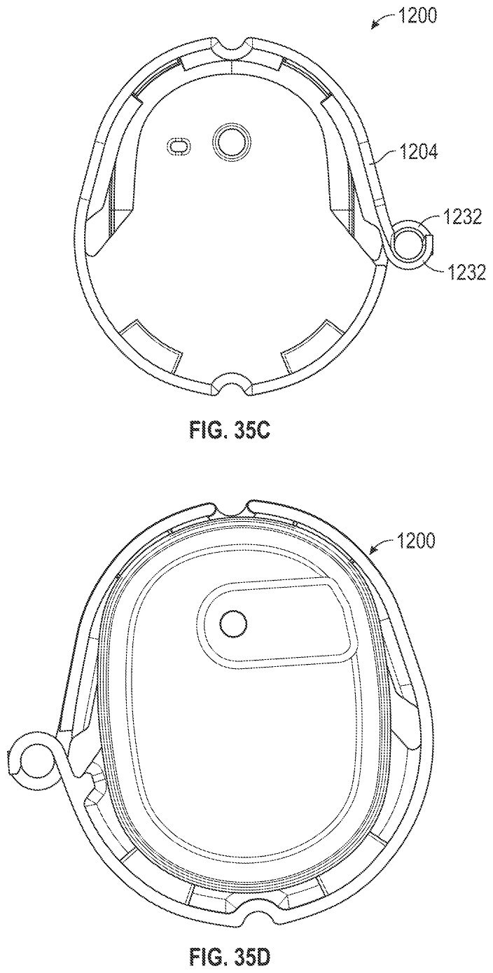

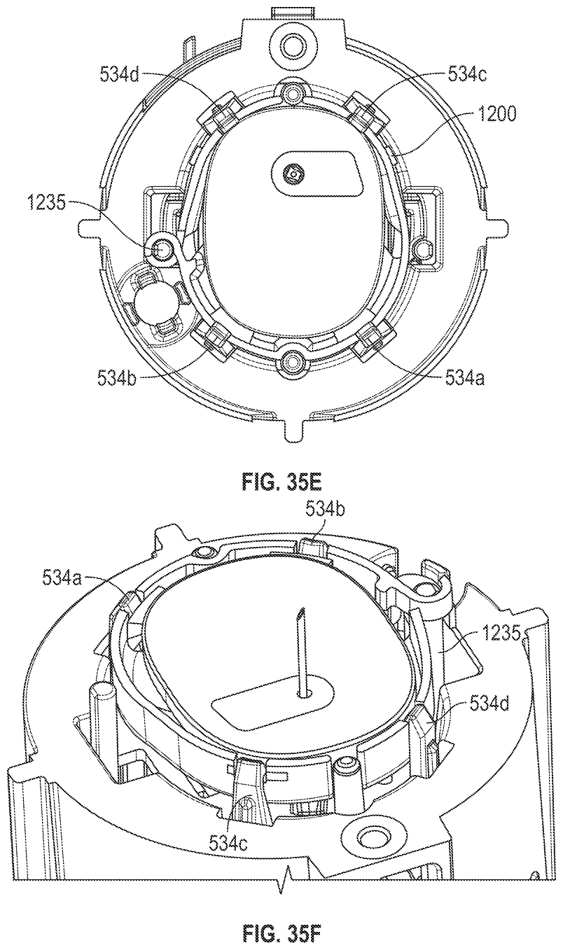

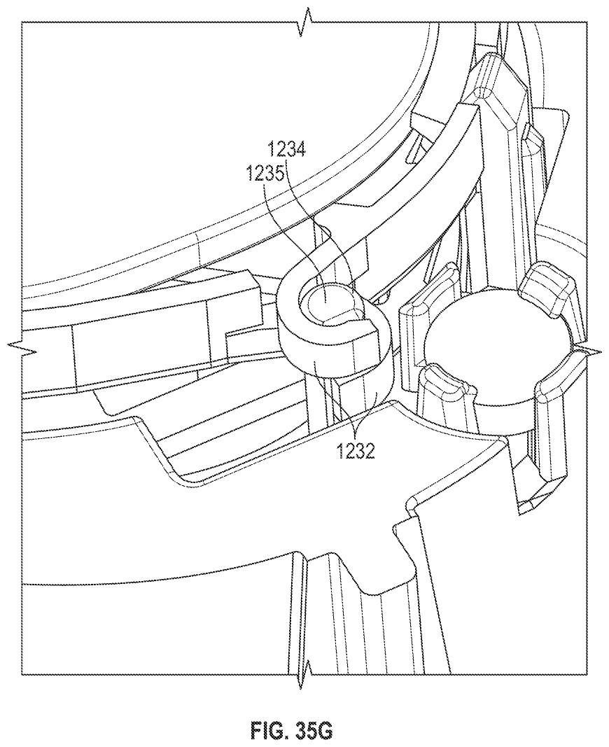

Other configurations of adaptor bodies may be utilized in examples. FIG. 35A illustrates an example of an adaptor body 1200. The adaptor body 1200 may include the features of other adaptor bodies disclosed herein unless stated otherwise.

Referring to FIGS. 35A-35D, for example, the adaptor body 1200 may include a retention area 1202 for receiving at least a portion of an on-skin wearable medical device. The retention area 1202, for example, may comprise a cavity for receiving at least the portion of the on-skin wearable medical device.

The adaptor body 1200 may include one or more walls that may bound the retention area 1202. The one or more walls may include one or more side walls 1204 that may bound the retention area 1202. The side walls 1204 may be configured to cover corresponding side surfaces 1008 (marked in FIG. 28) of the on-skin sensor assembly 1000.

The side walls 1204 may include an inner surface 1206 (marked in FIG. 35B) and an outer surface 1208. The inner surface 1206 may be configured to face towards the portion of the on-skin wearable medical device that may be positioned within the retention area 1202. The outer surface 1208 may be configured to face opposite the inner surface 1206 and may face radially outward from the on-skin wearable medical device.

The one or more side walls 1204 may have a spacing between the inner surface 1206 and the outer surface 1208 that may be configured to space an outer surface 1008 (marked in FIG. 28) of the on-skin wearable medical device from the outer surface 1208 of the one or more side walls 1204. A thickness of the one or more side walls 1204 may form the spacing, or the spacing may be provided in other manners.

The inner surface 1206 may be configured to contact the outer surface 1008 of the on-skin wearable medical device. The inner surface 1206 may be contoured to the shape of the outer surface 1008 of the on-skin wearable medical device to provide a contoured fit to the shape of the outer surface 1008. The outer surface 1208 of the adaptor body 1200 may have a different contour than the inner surface 1206 of the adaptor body 1200 in examples.

For example, the inner surface 1206 may form an inner perimeter 1210 (marked in FIG. 35B) of the adaptor body 1200. The outer surface 1208 may form an outer perimeter 1212 of the adaptor body 1200. The inner perimeter 1210 may have a different contour than the outer perimeter 1212. Such a feature may account for a difference in shape between the outer side surface 1008 of the on-skin sensor assembly 1000 and a shape to which the adaptor body 1200 is adapting the on-skin sensor assembly 1000. The inner perimeter 1210 may have a smaller diameter than the outer perimeter 1212 in examples.

The spacing between the inner surface 1206 and the outer surface 1208 may adapt the on-skin wearable medical device to fit an engagement portion of an applicator. For example, the engagement portion of the applicator may be configured to engage an on-skin sensor assembly having a larger diameter than the on-skin sensor assembly 1000. The spacing may compensate for the difference in diameters. The spacing may alternatively or in combination compensate for a difference in shape between the on-skin sensor assemblies.

The outer surface 1208 of the side walls 1204 may include a stabilization feature 1240 that may be configured to stabilize the adaptor body 1200 within an applicator. The stabilization feature 1240 may comprise a recess of the outer surface 1208 of the side walls 1204. The stabilization feature 1240 may have a contour and position similar to the stabilization feature 523 shown in FIG. 27 for example. As such, the stabilization feature 1240 may be utilized to stabilize the adaptor body 1200 in a similar manner as the stabilization feature 523 stabilizes the on-skin sensor assembly 508 within the applicator. Other configurations of stabilization features may be utilized as desired.

The one or more walls may include an upper wall 1214 (marked in FIG. 35A) that may bound the retention area 1202. The upper wall 1214 may be configured to cover a corresponding outer upper, top, or proximal surface 1010 (marked in FIG. 28) of the on-skin wearable medical device. The upper wall 1214 may include an inner surface 1216 (marked in FIG. 35B) and an outer surface 1218. The inner surface 1216 may be configured to face towards the portion of the on-skin wearable medical device that may be positioned within the retention area 1202. The outer surface 1218 may be configured to face opposite the inner surface 1216 and may face radially outward from the on-skin wearable medical device.

The upper wall 1214 may have a spacing between the inner surface 1216 and the outer surface 1218 that may be configured to space an outer upper, top, or proximal surface 1010 (marked in FIG. 28) of the on-skin wearable medical device from the outer surface 1218 of the upper wall 1214. The outer surface 1010 of the on-skin wearable medical device, for example, may comprise an outer upper surface 1010 of the housing 1002. A thickness of the upper wall 1214 may form the spacing, or the spacing may be provided in other manners. The upper wall 1214 may include an opening 1215 that may allow for insertion or retraction of an insertion element, such as a needle.

The inner surface 1216 may be configured to contact the outer surface 1010 of the on-skin wearable medical device. The inner surface 1216 may be contoured to the shape of the outer surface 1010 of the on-skin wearable medical device to provide a contoured fit to the shape of the outer surface 1010. The outer upper surface 1218 of the adaptor body 1200 may have a different contour than the inner surface 1216 of the adaptor body 1200 in examples.

The spacing between the inner surface 1216 and the outer surface 1218 may adapt the on-skin wearable medical device to fit an engagement portion of an applicator. For example, the engagement portion of the applicator may be configured to engage an on-skin sensor assembly having a larger height than the on-skin sensor assembly 1000. The spacing may compensate for the difference in height. The spacing may alternatively or in combination compensate for a difference in shape between the on-skin sensor assemblies.

The side walls 1204 may extend distally from the upper wall 1214 in examples. The side walls 1204 may extend distally from the outer edges or perimeter of the upper wall 1214 in examples. Other configurations may be provided in examples.

The adaptor body 1200 may include a retainer portion 1220 for retaining the on-skin wearable medical device to the adaptor body 1200. The retainer portion 1220 may have a variety of forms. In examples, the retainer portion 1220 may include one or more device couplers 1222 that may couple the on-skin wearable medical device to the adaptor body 1080.

The device couplers 1222 may have a variety of forms in examples. Referring to FIG. 35B, the device couplers 1222 may include protrusions 1224 that may be configured to engage the on-skin sensor assembly 1000. The protrusions 1224 may be configured to engage coupling features of the wearable housing 1002 of the on-skin sensor assembly 1000 that may have a different configuration than the coupling features shown in FIG. 28 for example. The coupling features, for example, may be positioned at end portions of the wearable housing 1002. Other locations may be utilized as desired. The protrusions 1224 may enter into the coupling features in the form of recesses in the wearable housing 1002. The device couplers 1222 may have other forms in examples. For example, the device couplers may comprise a protrusion or a recess, or one or more of a protrusion or recess. The device couplers may comprise an adhesive or may have another form.

The device couplers 1222 may comprise arms in examples. For example, the arms 1226 may extend circumferentially about the outer perimeter of the adaptor body 1200. The arms 1226 may extend from a hinge portion 1228 circumferentially to a respective end portion 1230 of the arm 1226. Each arm 1226 may extend circumferentially about a side surface of the wearable housing 1002. Each arm 1226 may include the protrusions 1224.

A respective hinge portion 1228 may allow a respective arm 1226 to deflect radially outward from the wearable housing 1002. The hinge portion 1228 may be configured to pivot about an axis extending transverse or perpendicular with a plane of the adaptor body 1200 (e.g., the plane of the upper wall 1214).

The arms 1226 may bound the retention area 1202 in examples. The arms 1226 may deflect radially outward from the retention area 1202 to allow for release of the on-skin wearable medical device from the retention area 1202 of the adaptor body 1200.

The arms 1226 of the device couplers 1222 may comprise the majority of the outer perimeter 1212 of the adaptor body 1200. The arms 1226 may comprise at least 60% or 70% or 80% of the outer perimeter 1212 in examples.

The device couplers 1222 may comprise releasable couplers configured to release the on-skin wearable medical device from the adaptor body 1200. The device couplers 1222, for example, may be configured to deflect to release the on-skin wearable medical device from the adaptor body 1200. The deflection may be in a radially outward direction to allow the device couplers 1222 to disengage from the on-skin wearable medical device.

In examples, the adaptor body 1200 may include one or more support portions 1232. The support portions 1232 may be configured to support the device couplers 1222 in a coupled configuration with the on-skin wearable medical device. The support portions 1232 may have a variety of forms and may comprise contact surfaces of the adaptor body 1200. The support portions 1232 may comprise hooks as shown in FIG. 35B. The hooks may be configured to extend around a contact surface 1234 of the applicator (marked in FIG. 35G) to press the device couplers 1222 towards the on-skin wearable medical device. The contact surface 1234 of the applicator may comprise at least one post that may engage the hooks. The device couplers 1222 accordingly may be held in the coupled configuration with the on-skin wearable medical device. The post may be configured to be withdrawn from the hooks to allow the device couplers 1222 to decouple from the portion of the on-skin wearable medical device. The post may be withdrawn during retraction of an insertion element or needle for guiding the transcutaneous analyte sensor into the skin of a host, as disclosed herein. The support portions 1232 may have other configurations in examples.

In examples, the device couplers 1222 may be biased to extend radially outward from the retention area 1202. As such, upon release of the support portions 1232, the device couplers 1222 may release from the on-skin wearable medical device.

FIG. 35C illustrates a top view of the adaptor body 1200 positioned upon the on-skin wearable medical device. FIG. 35D illustrates a bottom view of the adaptor body 1200 positioned upon the on-skin wearable medical device.

FIG. 35E illustrates the adaptor body 1200 coupled to the applicator. The engagement portion of the applicator, for example, the wearable retention elements 534a-d may engage the side walls 1204 of the adaptor body 1200. Contact surfaces of the side walls 1204 may be positioned in the same position that the coupling features 521 shown in FIG. 27 would engage the wearable retention elements 534a-d. The applicator includes at least one stabilizer 1235 having the contact surface 1234 that mates with the support portion 1232 of the adaptor body 1200. The stabilizer 1235 may comprise a post engaging the hooks of the support portions 1232. The hooks may wrap around the post. The contact between the stabilizer 1235 and the support portions 1232 of the adaptor body 1200 is shown in the perspective view of FIG. 35G. The contact may retain the device couplers 1222 in the closed configuration.

FIG. 35F illustrates the assembly prior to retraction of the needle carrier assembly 516. The needle carrier assembly 516 may be retracted in a deployment procedure according to methods disclosed herein. The engagement portion of the applicator in the form of the wearable retention elements 534a-d may be released from the adaptor body 1200 in a similar manner as disclosed herein regarding the on-skin sensor assembly 508 (e.g., FIGS. 15 and 16 illustrate an exemplary release).

The stabilizer 1235 having the contact surface for contacting the support portions 1232 of the adaptor body 1200 may be retracted. The post may be retracted from the position between the hooks of the support portions 1232. The post accordingly may no longer be between the hooks of the support portions 1232. The support portions 1232 accordingly may be unsupported by the contact surfaces and the device couplers 1222 may be configured to deflect radially outward. The hooks may release in opposite directions from each other to allow the device couplers 1222 to deflect radially outward and decouple from the on-skin wearable medical device. The outward deflection of the device couplers 1222 may allow the on-skin sensor assembly 1000 to release from the adaptor body 1200. The on-skin sensor assembly 1000 may be deployed to the skin of the host with an adhesive or other form of engagement with the skin, and thus may remain deployed to the skin upon removal of the applicator from the skin.

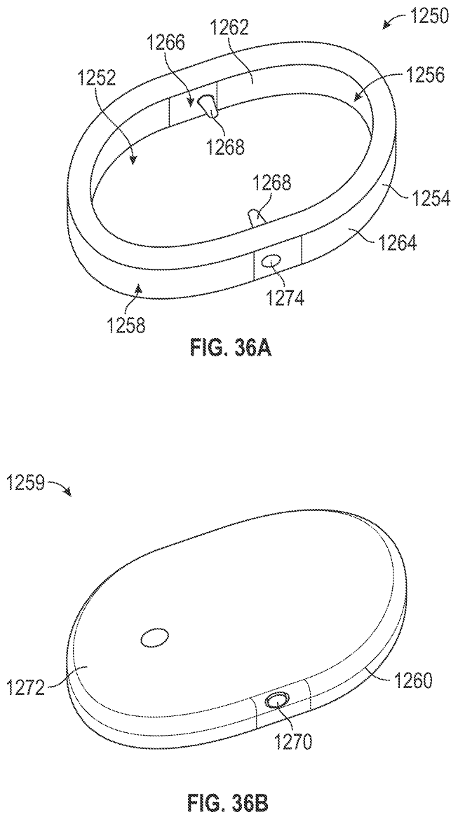

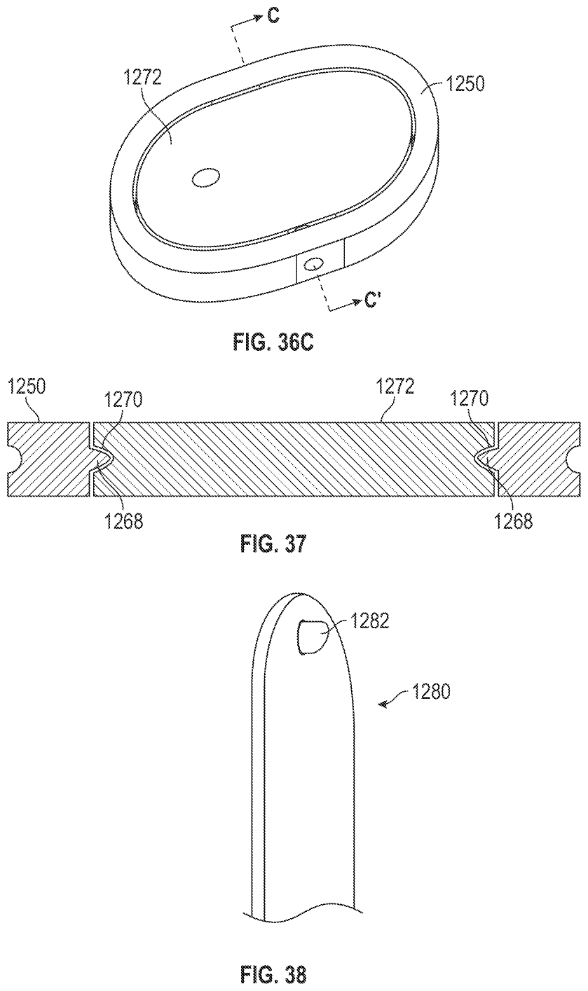

Other configurations of adaptor bodies may be utilized in examples. FIG. 36A illustrates an example of an adaptor body 1250. The adaptor body 1250 may include the features of other examples of adaptor bodies unless stated otherwise.

Referring to FIG. 36A, the adaptor body 1250 may include a retention area 1252 for receiving at least a portion of an on-skin wearable medical device. The retention area 1252, for example, may comprise a cavity for receiving at least the portion of the on-skin wearable medical device.

The adaptor body 1250 may include one or more walls that may bound the retention area 1252. The one or more walls may include one or more side walls 1254 that may bound the retention area 1252. The side walls 1254 may be configured to cover corresponding side surfaces 1008 (marked in FIG. 28) of the on-skin sensor assembly 1000.

The side walls 1254 may include an inner surface 1256 and an outer surface 1258. The inner surface 1256 may be configured to face towards the portion of the on-skin wearable medical device that may be positioned within the retention area 1252. The outer surface 1258 may be configured to face opposite the inner surface 1256 and may face radially outward from the on-skin wearable medical device.

The one or more side walls 1254 may have a spacing between the inner surface 1256 and the outer surface 1258 that may be configured to space an outer surface 1260 (marked in FIG. 36B) of the on-skin wearable medical device from the outer surface 1258 of the one or more side walls 1254. The outer surface 1260 of the on-skin wearable medical device, for example, may comprise an outer side surface 1260 of the housing 1272 of the on-skin wearable medical device. A thickness of the one or more side walls 1254 may form the spacing, or the spacing may be provided in other manners.

The inner surface 1256 may be configured to contact the outer surface 1260 of the on-skin wearable medical device. The inner surface 1256 may be contoured to the shape of the outer surface 1260 of the on-skin wearable medical device to provide a contoured fit to the shape of the outer surface 1260. The outer surface 1258 of the adaptor body 1250 may have a different contour than the inner surface 1256 of the adaptor body 1250 in examples.

For example, the inner surface 1256 may form an inner perimeter 1262 of the adaptor body 1250. The outer surface 1258 may form an outer perimeter 1264 of the adaptor body 1250. The inner perimeter 1262 may have a different contour than the outer perimeter 1264. Such a feature may account for a difference in shape between the outer side surface 1260 of the on-skin sensor assembly 1259 and a shape to which the adaptor body 1250 is adapting the on-skin sensor assembly 1259.

The spacing between the inner surface 1256 and the outer surface 1258 may adapt the on-skin wearable medical device to fit an engagement portion of an applicator. For example, the engagement portion of the applicator may be configured to engage an on-skin sensor assembly having a larger diameter than the on-skin sensor assembly 1259. The spacing may compensate for the difference in diameters. The spacing may alternatively or in combination compensate for a difference in shape between the on-skin sensor assemblies.

The adaptor body 1250 may comprise a ring extending about the retention area 1252.

The adaptor body 1250 may include a retainer portion 1266 for retaining the on-skin wearable medical device to the adaptor body 1250. The retainer portion 1266 may have a variety of forms. In examples, the retainer portion 1266 may include one or more device couplers 1268 that may couple the on-skin wearable medical device to the adaptor body 1250.

The device couplers 1268 may have a variety of forms in examples. Referring to FIG. 36A, the device couplers 1268 may include protrusions that may be configured to engage the on-skin sensor assembly 1259. The protrusions may be configured to engage the coupling features 1270 (marked in FIG. 36B) of the wearable housing 1272 of the on-skin sensor assembly 1259. The protrusions may enter into the coupling features 1270 in the form of recesses in the wearable housing 1272. The device couplers 1268 may have other forms in examples. For example, the device couplers may comprise a protrusion or a recess, or one or more of a protrusion or recess. The device couplers may comprise an adhesive or may have another form. A ball and groove arrangement (e.g., with the coupling features 1270 comprising a groove) may be utilized.

The device couplers 1268 may be positioned on the inner surface 1256 of the side walls 1254 and may be configured to extend radially inward towards the retention area 1252. The device couplers 1268 may have other positions in examples.

The device couplers 1268 may comprise releasable couplers configured to release the on-skin wearable medical device from the adaptor body 1250. The device couplers 1268 may be configured to release from the on-skin wearable medical device upon a sufficient force being applied to the device couplers 1268. The protrusions may disengage from the coupling features 1270. In an example in which the device couplers 1268 comprise recesses, protrusions of the wearable housing 1272 may disengage from the recesses of the adaptor body 1250 to provide for release of the adaptor body 1250.

Applicator couplers 1274 may be utilized for coupling the adaptor body 1250 to at least a portion of the applicator. The applicator couplers 1274 may comprise recesses that may engage protrusions of the applicator. The applicator couplers 1274 may have other forms, such as protrusions or an adhesive. One or more of a protrusion or recess may be provided in examples. As shown in FIG. 36A, the applicator couplers 1274 may be positioned upon the outer surface 1258 of the side walls 1254 or at another position as desired.

FIG. 36B illustrates a perspective view of an exemplary on-skin sensor assembly 1259 that may be utilized with the adaptor body 1250. The adaptor body 1250 may surround the wearable housing 1272 of the on-skin sensor assembly 1259.

FIG. 36C, for example, illustrates the adaptor body 1250 positioned about the wearable housing 1272. The device couplers 1268 may couple with the coupling features 1270. FIG. 37, for example, illustrates a cross sectional view along line C-C′ in FIG. 36C.

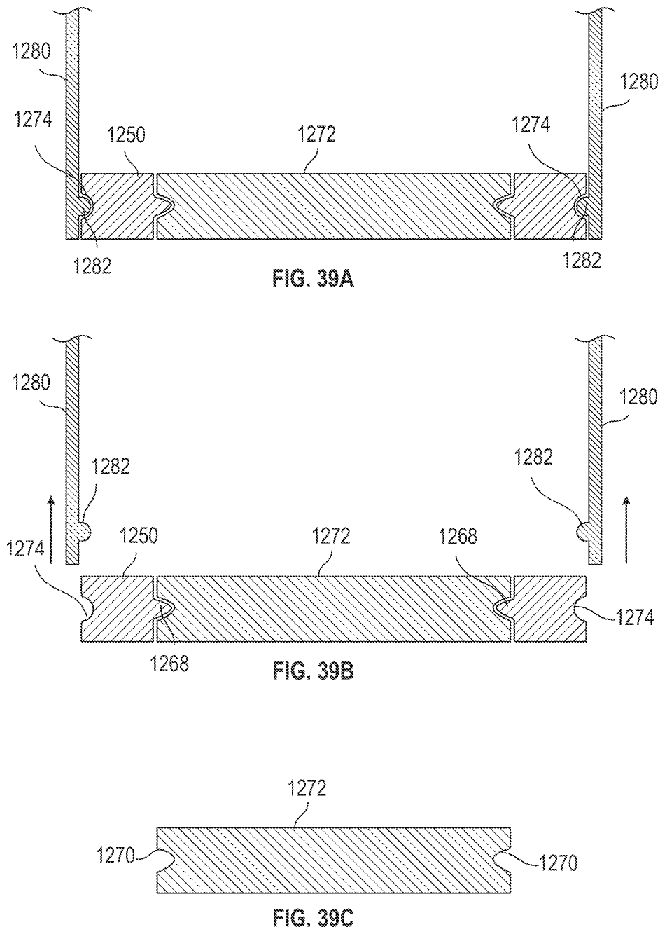

The engagement portion of the applicator may be configured to engage the adaptor body 1250. The engagement portion may comprise wearable retention and/or alignment elements as disclosed herein, or may have other forms. FIG. 38, for example, illustrates a configuration of an engagement portion 1280 comprising a retention element in the form of a protrusion 1282. The protrusions 1282 may be positioned upon arms of wearable retention and/or alignment elements as disclosed herein, or may have other forms. Other forms (e.g., recesses, adhesives) may be utilized as desired. The wearable retention and/or alignment elements may comprise a portion of a needle carrier assembly, as disclosed herein. The engagement portion 1280 may be configured to engage the applicator couplers 1274 as shown in the cross sectional view of FIG. 39A. The protrusions 1282 may engage the applicator couplers 1274, or another coupling configuration may result.

The adaptor body 1250 may adapt the on-skin sensor assembly 1259 to fit the engagement portion 1280.

The engagement portion 1280 may be configured to release from the adaptor body 1250 in examples. For example, referring to FIG. 39B, the engagement portion 1280 of the applicator may release upon being withdrawn from the adaptor body 1250. The protrusions 1282 may disengage from the applicator couplers 1274 upon a force pulling the protrusions 1282 from the applicator couplers 1274. The engagement portion 1280, for example, may deflect out of the applicator couplers 1274 for release. The arms of the wearable retention and/or alignment elements, for example, may deflect outward to allow the protrusions 1282 to release from the applicator couplers 1274. Other forms of release may be utilized as desired. In examples, the adaptor body 1250 may be removed from the wearable housing 1272 if desired. The on-skin wearable medical device may be released from the adaptor body 1250. FIG. 39C, for example, illustrates a resulting configuration. The device couplers 1268 may be decoupled from the coupling features 1270.

In examples, the adaptor body 1250 may remain engaged with the engagement portion 1280 upon deployment of the wearable housing 1272. For example, the wearable housing 1272 may disengage from the adaptor body 1250 and may remain on the host's skin. The adaptor body 1250 may remain engaged with the engagement portion 1280 and may be retracted from the host's skin along with the engagement portion 1280 of the applicator. The wearable housing 1272 may remain positioned upon the host's skin as represented in FIG. 39C for example.

In examples, the adaptor body 1250 may interface between the on-skin wearable medical device and the applicator, without the applicator being able to engage other configurations of on-skin wearable medical devices. For example, in a configuration as shown in FIGS. 36A-39C, the engagement portion 1280 may be unable to engage any other form of on-skin wearable medical device. The adaptor body 1250 yet may interface with the engagement portion 1280 by allowing the on-skin wearable medical device to fit the engagement portion 1280. Such a configuration may be beneficial to allow for multiple different sizes or other configurations of on-skin wearable medical devices to fit a single form of engagement portion. For example, referring to FIGS. 36A-39C, multiple different adaptor bodies may be utilized to fit multiple different configurations of on-skin wearable medical devices to the engagement portion 1280. Different diameters or shapes of adaptor bodies may be utilized to adapt multiple different diameters or shape of adaptor bodies with the same engagement portion 1280. Other configurations of the engagement portion and the on-skin wearable medical devices may be accounted for with the use of an adaptor body.

An applicator as disclosed herein (e.g., the applicators disclosed herein including applicators 500, 700, 900) may include an activation body 1300 (marked in FIG. 30B, for example) that may be utilized to electrically activate the on-skin wearable medical device or on-skin sensor assembly 1000. Features and operation of an activation body 1300 are disclosed in U.S. patent application Ser. No. 16/400,974, filed May 1, 2019, and published as U.S. Publication No. 2019/0342637 on Nov. 7, 2019, the entire contents of which are incorporated by reference for all purposes. The activation body 1300 may operate to activate a sensor electronics module 140 of the on-skin sensor assembly 1000 through remote sensing by the sensor electronics module 140. The sensor electronics module 140, for example, may include a proximity sensor that may detect the proximity of the activation body 1300. The proximity sensor may have the forms disclosed in U.S. Publication No. 2019/0342637, e.g., a Hall effect sensor, Reed switch, or other form of proximity sensor. The proximity sensor may be a magnetic field sensor for sensing a magnetic field of the activation body 1300. The activation body 1300, for example, may comprise a magnet that produces a magnetic field. A variation in the proximity of the proximity sensor to the activation body 1300 (e.g., a variation in the strength of the magnetic field produced by the activation body 1300 due to distance) may be sensed and may be utilized to activate the on-skin sensor assembly 1000.

The electrical activation may be from a lower power state to a higher power state in a manner disclosed in U.S. Publication No. 2019/0342637. The on-skin sensor assembly 1000 and sensor electronics module 140 may be held in a low power state or quiescent state when coupled to the applicator (e.g., the applicators disclosed herein including applicators 500, 700, 900). This is because the on-skin sensor assembly 1000 is not in use in such a configuration. The on-skin sensor assembly 1000 may conserve battery power in such a configuration. The on-skin sensor assembly 1000, upon deployment, is intended for use and thus is activatable to a higher power state for operation of the on-skin sensor assembly 1000 upon the host's skin. The distance between the on-skin sensor assembly 1000 and the activation body 1300 increases upon application of the on-skin sensor assembly 1000 when the applicator is removed from the on-skin sensor assembly 1000 positioned on the host's skin. The increase in distance is detected by the proximity sensor and the on-skin sensor assembly 1000 is thus activated to the higher power state for use.

Referring to FIG. 30B, the position of the activation body 1300 on the applicator may require a large activation body 1300 (e.g., a large magnet) to produce a sufficient magnetic field to be sensed by the sensor electronics module 140 of the on-skin sensor assembly 1000. Further, if the size of the on-skin sensor assembly 1000 is reduced, or if the presence of additional structure such as an adaptor or other configuration changes increase the distance between an activation body 1300 of the applicator and the sensor electronics module 140, then an even larger activation body 1300 (e.g., a larger magnet) may be required due to an increased distance between the activation body 1300 and the on-skin sensor assembly 1000.

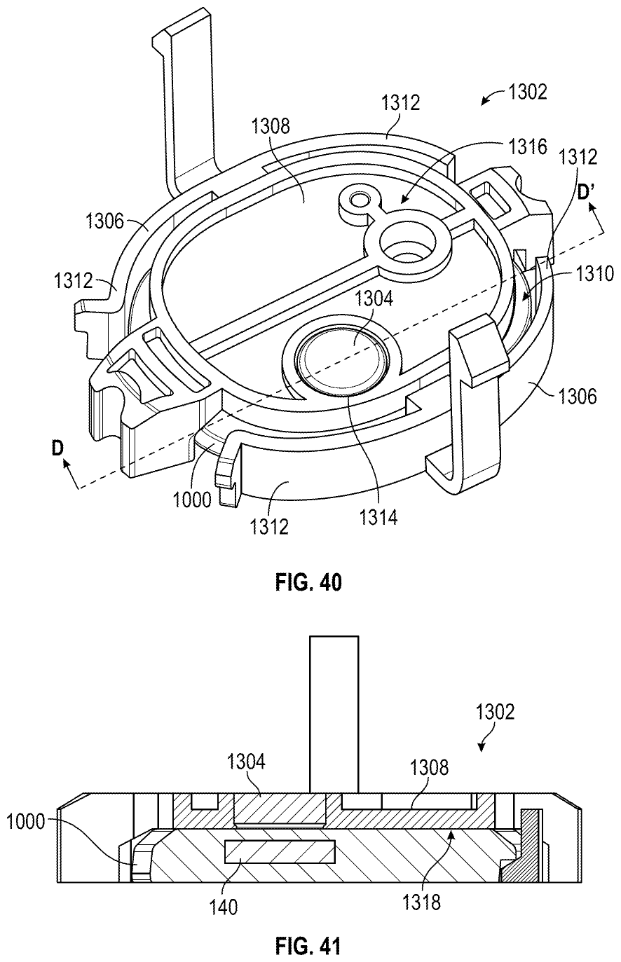

FIG. 40 illustrates an example in which an adaptor body 1302 includes an activation body 1304. The activation body 1304 operates in the same manner as the activation body 1300. The activation body 1304 may comprise a magnet.

The adaptor body 1302 is configured similarly as other examples of adaptor bodies disclosed herein. The adaptor body 1302 includes one or more walls 1306, 1308 bounding a retention area 1310 for receiving the on-skin sensor assembly 1000. The one or more walls include one or more side walls 1306, and an upper wall 1308. The adaptor body 1302 may include one or more device couplers 1312 that may couple the on-skin sensor assembly 1000 to the adaptor body 1302. The device couplers 1312 may comprise arms extending along side surfaces of the on-skin sensor assembly 1000 and may operate in a similar manner as the device couplers 1106 of FIGS. 31A-31C for example. Other configurations of device couplers as disclosed herein may be utilized.

The activation body 1304 may be positioned in or on the one or more walls 1306, 1308. The activation body 1304 in FIG. 40 is positioned on the upper wall 1308. The activation body 1304 is positioned in a receiver 1314 in the outer surface 1316 of the upper wall 1308 or may be positioned on another surface (e.g., an inner surface 1318 marked in FIG. 41). The receiver 1314 may comprise a cavity or may have another configuration. The activation body 1304 may be positioned on a side wall 1306 in examples. In examples, receiver 1314 or other portion of the adaptor body 1302 can include one or more securement features (e.g., snap-fit features, retaining tabs, or adhesive) to retain the activation body 1304 in the receiver.

The activation body 1304 may be positioned proximate the proximity sensor of the on-skin sensor assembly 1000 when the assembly 1000 is received within the adaptor body 1302. FIG. 41, for example, illustrates a cross sectional view of FIG. 40 along line D-D′ showing the activation body 1304 positioned above and proximate the sensor electronics 140 of the on-skin sensor assembly 1000 that may include the proximity sensor. The position of the activation body 1304 on the adaptor body 1302 may beneficially increase the proximity of the activation body 1304 to the proximity sensor of the on-skin sensor assembly 1000. A greater magnetic field may be provided for detection by the proximity sensor. An activation body 1304 that is smaller than the activation body 1300 may be utilized to provide a field or other signal for detection by the proximity sensor.

Any of the adaptor bodies disclosed herein may incorporate an activation body 1304 as disclosed herein. The activation body 1304 may comprise a magnet, or may have other forms depending on the form of proximity sensor utilized (e.g., a magnetic responsive material may be utilized in an example in which the proximity sensor includes a magnet; an electric field generator (e.g., a charged body) may be utilized; an optical indicator may be utilized for optical sensing, among others).

In operation, the activation body 1304 may operate in a similar manner as the activation body 1300. A distance between the activation body 1304 and the on-skin sensor assembly 1000 increases upon deployment of the on-skin sensor assembly 1000 to the skin of the host. The proximity sensor senses the removal of the activation body 1304 and causes the on-skin sensor assembly 1000 to electrically activate from the lower power state to the higher power state. Variations in the method of operation may be provided in examples.

Features of adaptor bodies may allow for interface of different configurations of on-skin wearable medical devices with a variety of configurations of applicators. In examples, the applicator may comprise a universal applicator, with the adaptor body interfacing to allow an on-skin wearable medical device to engage the universal applicator.

Features of examples disclosed herein may be utilized solely or in combination with any other system, apparatus, or method disclosed herein.

The above description presents the best mode contemplated for carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular examples disclosed. On the contrary, this invention covers all modifications and alternate constructions coming within the spirit and scope of the invention as generally expressed by the following claims, which particularly point out and distinctly claim the subject matter of the invention. While the disclosure has been illustrated and described in detail in the drawings and foregoing description, such illustration and description are to be considered illustrative or exemplary and not restrictive.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the examples.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and ‘one or more’ to introduce claim recitations. However, the use of such phrases should not be construed to imply that the introduction of a claim recitation by the indefinite articles ‘a’ or ‘an’ limits any particular claim containing such introduced claim recitation to examples containing only one such recitation, even when the same claim includes the introductory phrases ‘one or more’ or ‘at least one’ and indefinite articles such as ‘a’ or ‘an’ (e.g., ‘a’ and/or ‘an’ should typically be interpreted to mean ‘at least one’ or ‘one or more’); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples described herein, but rather to also cover all modification and alternatives coming with the true scope and spirit of the invention.

1. An apparatus comprising:
an adaptor body configured to interface between at least a portion of an on-skin wearable medical device and an applicator of the on-skin wearable medical device.

2. The apparatus of claim 1, wherein the adaptor body is configured to interface between at least the portion of the on-skin wearable medical device having a first configuration and a portion of the applicator configured to engage a second configuration of at least a portion of an on-skin wearable medical device that is different than the first configuration.

3. The apparatus of claim 2, wherein the second configuration differs from the first configuration in one or more of a shape or a size.

4. The apparatus of claim 2, wherein:
the on-skin wearable medical device having at least the portion with the first configuration is a first on-skin wearable medical device, and the on-skin wearable medical device having at least the portion with the second configuration is a second on-skin wearable medical device,
the first configuration comprises a configuration of a first wearable housing of the first on-skin wearable medical device, and
the second configuration comprises a configuration of a second wearable housing of the second on-skin wearable medical device.

5. The apparatus of claim 2, wherein the on-skin wearable medical device having at least the portion with the first configuration is the same on-skin wearable medical device that has at least the portion with the second configuration.

6. The apparatus of claim 1, wherein:
the on-skin wearable medical device is a first on-skin wearable medical device, and
the adaptor body is configured to interface between a wearable housing of the first on-skin wearable medical device that is smaller than a wearable housing of a second on-skin wearable medical device that the applicator is configured to engage.

7. The apparatus of claim 6, wherein the wearable housing of the first on-skin wearable medical device has a smaller diameter or a smaller height than the wearable housing of the second on-skin wearable medical device.

8. The apparatus of claim 1, wherein the adaptor body is configured to adapt at least the portion of the on-skin wearable medical device to fit an engagement portion of the applicator.

9. The apparatus of claim 1, wherein the adaptor body includes a retention area for receiving at least the portion of the on-skin wearable medical device.

10. The apparatus of claim 1, wherein the adaptor body includes a cavity for receiving at least the portion of the on-skin wearable medical device.

11.-24. (canceled)

25. The apparatus of claim 1, wherein the adaptor body includes a retainer portion for retaining at least the portion of the on-skin wearable medical device to the adaptor body.

26. The apparatus of claim 1, further comprising one or more device couplers for coupling the on-skin wearable medical device to the adaptor body.

27. The apparatus of claim 26, wherein the one or more device couplers comprise an adhesive.

28. The apparatus of claim 26, wherein the one or more device couplers comprise one or more of a protrusion or a recess configured to engage at least the portion of the on-skin wearable medical device.

29. The apparatus of claim 26, wherein the one or more device couplers are configured to deflect.

30.-38. (canceled)

39. The apparatus of claim 1, wherein the adaptor body includes an ejection portion configured to allow at least the portion of the on-skin wearable medical device to eject from the adaptor body.

40. (canceled)

41. The apparatus of claim 1, wherein the on-skin wearable medical device includes a transcutaneous analyte sensor, and the adaptor body is configured to be positioned within a housing of the applicator.

42.-45. (canceled)

46. A system comprising:
an applicator of an on-skin wearable medical device; and
an adaptor body configured to interface between at least a portion of the on-skin wearable medical device and the applicator of the on-skin wearable medical device.

47. The system of claim 46, wherein the applicator includes an engagement portion configured to engage the adaptor body.

48. (canceled)

49. The system of claim 47, wherein the on-skin wearable medical device is a first on-skin wearable medical device, and the engagement portion is configured to engage a second on-skin wearable medical device having a different configuration than the first on-skin wearable medical device.

50.-52. (canceled)

53. The system of claim 46, wherein the adaptor body includes a retention area for receiving at least the portion of the on-skin wearable medical device.

54.-62. (canceled)

63. The system of claim 46, wherein:
the adaptor body includes an ejection portion configured to allow at least the portion of the on-skin wearable medical device to eject from the adaptor body; and
the applicator includes an ejection actuator configured to eject the on-skin wearable medical device from the adaptor body.

64.-70. (canceled)

71. A method comprising:
utilizing an applicator to apply an on-skin wearable medical device to a skin of a host, an adaptor body interfacing between at least a portion of the on-skin wearable medical device and the applicator.

72. The method of claim 71, wherein the on-skin wearable medical device comprises a coupling mismatch with the applicator.

73. The method of claim 71, wherein the adaptor body adapts at least the portion of the on-skin wearable medical device to fit an engagement portion of the applicator.

74. The method of claim 71, wherein the on-skin wearable medical device is a first on-skin wearable medical device, and the applicator has an engagement portion configured to engage a second on-skin wearable medical device having a different configuration than the first on-skin wearable medical device.

75.-85. (canceled)
